SEQUENCE LISTING (251 sequences)

SEQ ID NO: 1 - protein, 15 aa, Artificial
  SIV gag peptide 1
MGVRNSVLSGKKADE

SEQ ID NO: 2 - protein, 15 aa, Artificial
  SIV gag peptide 2
NSVLSGKKADELEKI

SEQ ID NO: 3 - protein, 15 aa, Artificial
  SIV gag peptide 3
SGKKADELEKIRLRP

SEQ ID NO: 4 - protein, 15 aa, Artificial
  SIV gag peptide 4
ADELEKIRLRPNGKK

SEQ ID NO: 5 - protein, 15 aa, Artificial
  SIV gag peptide 5
EKIRLRPNGKKKYML

SEQ ID NO: 6 - protein, 15 aa, Artificial
  SIV gag peptide 6
LRPNGKKKYMLKHVV

SEQ ID NO: 7 - protein, 15 aa, Artificial
  SIV gag peptide 7
GKKKYMLKHVVWAAN

SEQ ID NO: 8 - protein, 15 aa, Artificial
  SIV gag peptide 8
YMLKHVVWAANELDR

SEQ ID NO: 9 - protein, 15 aa, Artificial
  SIV gag peptide 9
HVVWAANELDRFGLA

SEQ ID NO: 10 - protein, 15 aa, Artificial
  SIV gag peptide 10
AANELDRFGLAESLL

SEQ ID NO: 11 - protein, 15 aa, Artificial
  SIV gag peptide 11
LDRFGLAESLLENKE

SEQ ID NO: 12 - protein, 15 aa, Artificial
  SIV gag peptide 12
GLAESLLENKEGCQK

SEQ ID NO: 13 - protein, 15 aa, Artificial
  SIV gag peptide 13
SLLENKEGCQKILSV

SEQ ID NO: 14 - protein, 15 aa, Artificial
  SIV gag peptide 14
NKEGCQKILSVLAPL

SEQ ID NO: 15 - protein, 15 aa, Artificial
  SIV gag peptide 15
CQKILSVLAPLVPTG

SEQ ID NO: 16 - protein, 15 aa, Artificial
  SIV gag peptide 16
LSVLAPLVPTGSENL

SEQ ID NO: 17 - protein, 15 aa, Artificial
  SIV gag peptide 17
LSVLAPLVPTGSENL

SEQ ID NO: 18 - protein, 15 aa, Artificial
  SIV gag peptide 18
PTGSENLKSLYNTVC

SEQ ID NO: 19 - protein, 15 aa, Artificial
  SIV gag peptide 19
ENLKSLYNTVCVIWC

SEQ ID NO: 20 - protein, 15 aa, Artificial
  SIV gag peptide 20
SLYNTVCVIWCIHAE

SEQ ID NO: 21 - protein, 15 aa, Artificial
  SIV gag peptide 21
TVCVIWCIHAEEKVK

SEQ ID NO: 22 - protein, 15 aa, Artificial
  SIV gag peptide 22
IWCIHAEEKVKHTEE

SEQ ID NO: 23 - protein, 15 aa, Artificial
  SIV gag peptide 23
HAEEKVKHTEEAKQI

SEQ ID NO: 24 - protein, 15 aa, Artificial
  SIV gag peptide 24
KVKHTEEAKQIVQRH

SEQ ID NO: 25 - protein, 15 aa, Artificial
  SIV gag peptide 25
TEEAKQIVQRHLVVE

SEQ ID NO: 26 - protein, 15 aa, Artificial
  SIV gag peptide 26
KQIVQRHLVVETGTT

SEQ ID NO: 27 - protein, 15 aa, Artificial
  SIV gag peptide 27
QRHLVVETGTTETMP

SEQ ID NO: 28 - protein, 15 aa, Artificial
  SIV gag peptide 28
VVETGTTETMPKTSR

SEQ ID NO: 29 - protein, 15 aa, Artificial
  SIV gag peptide 29
GTTETMPKTSRPTAP

SEQ ID NO: 30 - protein, 15 aa, Artificial
  SIV gag peptide 30
TMPKTSRPTAPSSGR

SEQ ID NO: 31 - protein, 15 aa, Artificial
  SIV gag peptide 31
TSRPTAPSSGRGGNY

SEQ ID NO: 32 - protein, 15 aa, Artificial
  SIV gag peptide 32
TAPSSGRGGNYPVQQ

SEQ ID NO: 33 - protein, 15 aa, Artificial
  SIV gag peptide 33
SGRGGNYPVQQIGGN

SEQ ID NO: 34 - protein, 15 aa, Artificial
  SIV gag peptide 34
GNYPVQQIGGNYVHL

SEQ ID NO: 35 - protein, 15 aa, Artificial
  SIV gag peptide 35
VQQIGGNYVHLPLSP

SEQ ID NO: 36 - protein, 15 aa, Artificial
  SIV gag peptide 36
GGNYVHLPLSPRTLN

SEQ ID NO: 37 - protein, 15 aa, Artificial
  SIV gag peptide 37
VHLPLSPRTLNAWVK

SEQ ID NO: 38 - protein, 15 aa, Artificial
  SIV gag peptide 38
LSPRTLNAWVKLIEE

SEQ ID NO: 39 - protein, 15 aa, Artificial
  SIV gag peptide 39
TLNAWVKLIEEKKFG

SEQ ID NO: 40 - protein, 15 aa, Artificial
  SIV gag peptide 40
WVKLIEEKKFGAEVV

SEQ ID NO: 41 - protein, 15 aa, Artificial
  SIV gag peptide 41
IEEKKFGAEVVPGFQ

SEQ ID NO: 42 - protein, 15 aa, Artificial
  SIV gag peptide 42
KFGAEVVPGFQALSE

SEQ ID NO: 43 - protein, 15 aa, Artificial
  SIV gag peptide 43
EVVPGFQALSEGCTP

SEQ ID NO: 44 - protein, 15 aa, Artificial
  SIV gag peptide 44
GFQALSEGCTPYDIN

SEQ ID NO: 45 - protein, 15 aa, Artificial
  SIV gag peptide 45
LSEGCTPYDINQMLN

SEQ ID NO: 46 - protein, 15 aa, Artificial
  SIV gag peptide 46
CTPYDINQMLNCVGD

SEQ ID NO: 47 - protein, 15 aa, Artificial
  SIV gag peptide 47
DINQMLNCVGDHQAA

SEQ ID NO: 48 - protein, 15 aa, Artificial
  SIV gag peptide 48
MLNCVGDHQAAMQII

SEQ ID NO: 49 - protein, 15 aa, Artificial
  SIV gag peptide 49
VGDHQAAMQIIRDII

SEQ ID NO: 50 - protein, 15 aa, Artificial
  SIV gag peptide 50
QAAMQIIRDIINEEA

SEQ ID NO: 51 - protein, 15 aa, Artificial
  SIV gag peptide 51
QIIRDIINEEAADWD

SEQ ID NO: 52 - protein, 15 aa, Artificial
  SIV gag peptide 52
DIINEEAADWDLQHP

SEQ ID NO: 53 - protein, 15 aa, Artificial
  SIV gag peptide 53
EEAADWDLQHPQPAP

SEQ ID NO: 54 - protein, 15 aa, Artificial
  SIV gag peptide 54
DWDLQHPQPAPQQGQ

SEQ ID NO: 55 - protein, 15 aa, Artificial
  SIV gag peptide 55
QHPQPAPQQGQLREP

SEQ ID NO: 56 - protein, 15 aa, Artificial
  SIV gag peptide 56
PAPQQGQLREPSGSD

SEQ ID NO: 57 - protein, 15 aa, Artificial
  SIV gag peptide 57
QGQLREPSGSDIAGT

SEQ ID NO: 58 - protein, 15 aa, Artificial
  SIV gag peptide 58
REPSGSDIAGTTSSV

SEQ ID NO: 59 - protein, 15 aa, Artificial
  SIV gag peptide 59
GSDIAGTTSSVDEQI

SEQ ID NO: 60 - protein, 15 aa, Artificial
  SIV gag peptide 60
AGTTSSVDEQIQWMY

SEQ ID NO: 61 - protein, 15 aa, Artificial
  SIV gag peptide 61
SSVDEQIQWMYRQQN

SEQ ID NO: 62 - protein, 15 aa, Artificial
  SIV gag peptide 62
EQIQWMYRQQNPIPV

SEQ ID NO: 63 - protein, 15 aa, Artificial
  SIV gag peptide 63
WMYRQQNPIPVGNIY

SEQ ID NO: 64 - protein, 15 aa, Artificial
  SIV gag peptide 64
QQNPIPVGNIYRRWI

SEQ ID NO: 65 - protein, 15 aa, Artificial
  SIV gag peptide 65
IPVGNIYRRWIQLGL

SEQ ID NO: 66 - protein, 15 aa, Artificial
  SIV gag peptide 66
NIYRRWIQLGLQKCV

SEQ ID NO: 67 - protein, 15 aa, Artificial
  SIV gag peptide 67
RWIQLGLQKCVRMYN

SEQ ID NO: 68 - protein, 15 aa, Artificial
  SIV gag peptide 68
LGLQKCVRMYNPTNI

SEQ ID NO: 69 - protein, 15 aa, Artificial
  SIV gag peptide 69
KCVRMYNPTNILDVK

SEQ ID NO: 70 - protein, 15 aa, Artificial
  SIV gag peptide 70
MYNPTNILDVKQGPK

SEQ ID NO: 71 - protein, 15 aa, Artificial
  SIV gag peptide 71
TNILDVKQGPKEPFQ

SEQ ID NO: 72 - protein, 15 aa, Artificial
  SIV gag peptide 72
DVKQGPKEPFQSYVD

SEQ ID NO: 73 - protein, 15 aa, Artificial
  SIV gag peptide 73
GPKEPFQSYVDRFYK

SEQ ID NO: 74 - protein, 15 aa, Artificial
  SIV gag peptide 74
PFQSYVDRFYKSLRA

SEQ ID NO: 75 - protein, 15 aa, Artificial
  SIV gag peptide 75
YVDRFYKSLRAEQTD

SEQ ID NO: 76 - protein, 15 aa, Artificial
  SIV gag peptide 76
FYKSLRAEQTDAAVK

SEQ ID NO: 77 - protein, 15 aa, Artificial
  SIV gag peptide 77
LRAEQTDAAVKNWMT

SEQ ID NO: 78 - protein, 15 aa, Artificial
  SIV gag peptide 78
QTDAAVKNWMTQTLL

SEQ ID NO: 79 - protein, 15 aa, Artificial
  SIV gag peptide 79
AVKNWMTQTLLIQNA

SEQ ID NO: 80 - protein, 15 aa, Artificial
  SIV gag peptide 80
WMTQTLLIQNANPDC

SEQ ID NO: 81 - protein, 15 aa, Artificial
  SIV gag peptide 81
TLLIQNANPDCKLVL

SEQ ID NO: 82 - protein, 15 aa, Artificial
  SIV gag peptide 82
QNANPDCKLVLKGLG

SEQ ID NO: 83 - protein, 15 aa, Artificial
  SIV gag peptide 83
PDCKLVLKGLGVNPT

SEQ ID NO: 84 - protein, 15 aa, Artificial
  SIV gag peptide 84
LVLKGLGVNPTLEEM

SEQ ID NO: 85 - protein, 15 aa, Artificial
  SIV gag peptide 85
GLGVNPTLEEMLTAC

SEQ ID NO: 86 - protein, 15 aa, Artificial
  SIV gag peptide 86
NPTLEEMLTACQGVG

SEQ ID NO: 87 - protein, 15 aa, Artificial
  SIV gag peptide 87
EEMLTACQGVGGPGQ

SEQ ID NO: 88 - protein, 15 aa, Artificial
  SIV gag peptide 88
TACQGVGGPGQKARL

SEQ ID NO: 89 - protein, 15 aa, Artificial
  SIV gag peptide 89
GVGGPGQKARLMAEA

SEQ ID NO: 90 - protein, 15 aa, Artificial
  SIV gag peptide 90
PGQKARLMAEALKEA

SEQ ID NO: 91 - protein, 15 aa, Artificial
  SIV gag peptide 91
ARLMAEALKEALAPV

SEQ ID NO: 92 - protein, 15 aa, Artificial
  SIV gag peptide 92
AEALKEALAPVPIPF

SEQ ID NO: 93 - protein, 15 aa, Artificial
  SIV gag peptide 93
KEALAPVPIPFAAAQ

SEQ ID NO: 94 - protein, 15 aa, Artificial
  SIV gag peptide 94
APVPIPFAAAQQRGP

SEQ ID NO: 95 - protein, 15 aa, Artificial
  SIV gag peptide 95
IPFAAAQQRGPRKPI

SEQ ID NO: 96 - protein, 15 aa, Artificial
  SIV gag peptide 96
AAQQRGPRKPIKCWN

SEQ ID NO: 97 - protein, 15 aa, Artificial
  SIV gag peptide 97
RGPRKPIKCWNCGKE

SEQ ID NO: 98 - protein, 15 aa, Artificial
  SIV gag peptide 98
KPIKCWNCGKEGHSA

SEQ ID NO: 99 - protein, 15 aa, Artificial
  SIV gag peptide 99
CWNCGKEGHSARQCR

SEQ ID NO: 100 - protein, 15 aa, Artificial
  SIV gag peptide 100
GKEGHSARQCRAPRR

SEQ ID NO: 101 - protein, 15 aa, Artificial
  SIV gag peptide 101
HSARQCRAPRRQGCW

SEQ ID NO: 102 - protein, 15 aa, Artificial
  SIV gag peptide 102
QCRAPRRQGCWKCGK

SEQ ID NO: 103 - protein, 15 aa, Artificial
  SIV gag peptide 103
PRRQGCWKCGKMDHV

SEQ ID NO: 104 - protein, 15 aa, Artificial
  SIV gag peptide 104
GCWKCGKMDHVMAKC

SEQ ID NO: 105 - protein, 15 aa, Artificial
  SIV gag peptide 105
CGKMDHVMAKCPDRQ

SEQ ID NO: 106 - protein, 15 aa, Artificial
  SIV gag peptide 106
DHVMAKCPDRQAGFL

SEQ ID NO: 107 - protein, 15 aa, Artificial
  SIV gag peptide 107
AKCPDRQAGFLGLGP

SEQ ID NO: 108 - protein, 15 aa, Artificial
  SIV gag peptide 108
DRQAGFLGLGPWGKK

SEQ ID NO: 109 - protein, 15 aa, Artificial
  SIV gag peptide 109
GFLGLGPWGKKPRNF

SEQ ID NO: 110 - protein, 15 aa, Artificial
  SIV gag peptide 110
LGPWGKKPRNFPMAQ

SEQ ID NO: 111 - protein, 15 aa, Artificial
  SIV gag peptide 111
GKKPRNFPMAQVHQG

SEQ ID NO: 112 - protein, 15 aa, Artificial
  SIV gag peptide 112
RNFPMAQVHQGLMPT

SEQ ID NO: 113 - protein, 15 aa, Artificial
  SIV gag peptide 113
MAQVHQGLMPTAPPE

SEQ ID NO: 114 - protein, 15 aa, Artificial
  SIV gag peptide 114
HQGLMPTAPPEDPAV

SEQ ID NO: 115 - protein, 15 aa, Artificial
  SIV gag peptide 115
MPTAPPEDPAVDLLK

SEQ ID NO: 116 - protein, 15 aa, Artificial
  SIV gag peptide 116
PPEDPAVDLLKNYMQ

SEQ ID NO: 117 - protein, 15 aa, Artificial
  SIV gag peptide 117
PAVDLLKNYMQLGKQ

SEQ ID NO: 118 - protein, 15 aa, Artificial
  SIV gag peptide 118
LLKNYMQLGKQQREK

SEQ ID NO: 119 - protein, 15 aa, Artificial
  SIV gag peptide 119
YMQLGKQQREKQRES

SEQ ID NO: 120 - protein, 15 aa, Artificial
  SIV gag peptide 120
GKQQREKQRESREKP

SEQ ID NO: 121 - protein, 15 aa, Artificial
  SIV gag peptide 121
REKQRESREKPYKEV

SEQ ID NO: 122 - protein, 15 aa, Artificial
  SIV gag peptide 122
RESREKPYKEVTEDL

SEQ ID NO: 123 - protein, 15 aa, Artificial
  SIV gag peptide 123
EKPYKEVTEDLLHLN

SEQ ID NO: 124 - protein, 15 aa, Artificial
  SIV gag peptide 124
KEVTEDLLHLNSLFG

SEQ ID NO: 125 - protein, 14 aa, Artificial
  SIV gag peptide 125
EDLLHLNSLFGGDQ

SEQ ID NO: 126 - protein, 15 aa, Artificial
  HIV-1 gag peptide 1
MGARASVLSGGELDR

SEQ ID NO: 127 - protein, 15 aa, Artificial
  HIV-1 gag peptide 2
ASVLSGGELDRWEKI

SEQ ID NO: 128 - protein, 15 aa, Artificial
  HIV-1 gag peptide 3
SGGELDRWEKIRLRP

SEQ ID NO: 129 - protein, 15 aa, Artificial
  HIV-1 gag peptide 4
LDRWEKIRLRPGGKK

SEQ ID NO: 130 - protein, 15 aa, Artificial
  HIV-1 gag peptide 5
EKIRLRPGGKKKYKL

SEQ ID NO: 131 - protein, 15 aa, Artificial
  HIV-1 gag peptide 6
LRPGGKKKYKLKHIV

SEQ ID NO: 132 - protein, 15 aa, Artificial
  HIV-1 gag peptide 7
GKKKYKLKHIVWASR

SEQ ID NO: 133 - protein, 15 aa, Artificial
  HIV-1 gag peptide 8
YKLKHIVWASRELER

SEQ ID NO: 134 - protein, 15 aa, Artificial
  HIV-1 gag peptide 9
HIVWASRELERFAVN

SEQ ID NO: 135 - protein, 15 aa, Artificial
  HIV-1 gag peptide 10
ASRELERFAVNPGLL

SEQ ID NO: 136 - protein, 15 aa, Artificial
  HIV-1 gag peptide 11
ELERFAVNPGLLETS

SEQ ID NO: 137 - protein, 15 aa, Artificial
  HIV-1 gag peptide 12
FAVNPGLLETSEGCR

SEQ ID NO: 138 - protein, 15 aa, Artificial
  HIV-1 gag peptide 13
PGLLETSEGCRQILG

SEQ ID NO: 139 - protein, 15 aa, Artificial
  HIV-1 gag peptide 14
ETSEGCRQILGQLQP

SEQ ID NO: 140 - protein, 15 aa, Artificial
  HIV-1 gag peptide 15
GCRQILGQLQPSLQT

SEQ ID NO: 141 - protein, 15 aa, Artificial
  HIV-1 gag peptide 16
ILGQLQPSLQTGSEE

SEQ ID NO: 142 - protein, 15 aa, Artificial
  HIV-1 gag peptide 17
LQPSLQTGSEELRSL

SEQ ID NO: 143 - protein, 15 aa, Artificial
  HIV-1 gag peptide 18
LQTGSEELRSLYNTV

SEQ ID NO: 144 - protein, 15 aa, Artificial
  HIV-1 gag peptide 19
SEELRSLYNTVATLY

SEQ ID NO: 145 - protein, 15 aa, Artificial
  HIV-1 gag peptide 20
RSLYNTVATLYCVHQ

SEQ ID NO: 146 - protein, 15 aa, Artificial
  HIV-1 gag peptide 21
NTVATLYCVHQRIEV

SEQ ID NO: 147 - protein, 15 aa, Artificial
  HIV-1 gag peptide 22
TLYCVHQRIEVKDTK

SEQ ID NO: 148 - protein, 15 aa, Artificial
  HIV-1 gag peptide 23
VHQRIEVKDTKEALE

SEQ ID NO: 149 - protein, 15 aa, Artificial
  HIV-1 gag peptide 24
IEVKDTKEALEKIEE

SEQ ID NO: 150 - protein, 15 aa, Artificial
  HIV-1 gag peptide 25
DTKEALEKIEEEQNK

SEQ ID NO: 151 - protein, 15 aa, Artificial
  HIV-1 gag peptide 26
ALEKIEEEQNKSKKK

SEQ ID NO: 152 - protein, 15 aa, Artificial
  HIV-1 gag peptide 27
IEEEQNKSKKKAQQA

SEQ ID NO: 153 - protein, 15 aa, Artificial
  HIV-1 gag peptide 28
QNKSKKKAQQAAADT

SEQ ID NO: 154 - protein, 15 aa, Artificial
  HIV-1 gag peptide 29
KKKAQQAAADTGNSS

SEQ ID NO: 155 - protein, 15 aa, Artificial
  HIV-1 gag peptide 30
QQAAADTGNSSQVSQ

SEQ ID NO: 156 - protein, 15 aa, Artificial
  HIV-1 gag peptide 31
ADTGNSSQVSQNYPI

SEQ ID NO: 157 - protein, 15 aa, Artificial
  HIV-1 gag peptide 32
NSSQVSQNYPIVQNL

SEQ ID NO: 158 - protein, 15 aa, Artificial
  HIV-1 gag peptide 33
VSQNYPIVQNLQGQM

SEQ ID NO: 159 - protein, 15 aa, Artificial
  HIV-1 gag peptide 34
YPIVQNLQGQMVHQA

SEQ ID NO: 160 - protein, 15 aa, Artificial
  HIV-1 gag peptide 35
QNLQGQMVHQAISPR

SEQ ID NO: 161 - protein, 15 aa, Artificial
  HIV-1 gag peptide 36
GQMVHQAISPRTLNA

SEQ ID NO: 162 - protein, 15 aa, Artificial
  HIV-1 gag peptide 37
HQAISPRTLNAWVKV

SEQ ID NO: 163 - protein, 15 aa, Artificial
  HIV-1 gag peptide 38
SPRTLNAWVKVVEEK

SEQ ID NO: 164 - protein, 15 aa, Artificial
  HIV-1 gag peptide 39
LNAWVKVVEEKAFSP

SEQ ID NO: 165 - protein, 15 aa, Artificial
  HIV-1 gag peptide 40
VKVVEEKAFSPEVIP

SEQ ID NO: 166 - protein, 15 aa, Artificial
  HIV-1 gag peptide 41
EEKAFSPEVIPMFSA

SEQ ID NO: 167 - protein, 15 aa, Artificial
  HIV-1 gag peptide 42
FSPEVIPMFSALSEG

SEQ ID NO: 168 - protein, 15 aa, Artificial
  HIV-1 gag peptide 43
VIPMFSALSEGATPQ

SEQ ID NO: 169 - protein, 15 aa, Artificial
  HIV-1 gag peptide 44
FSALSEGATPQDLNT

SEQ ID NO: 170 - protein, 15 aa, Artificial
  HIV-1 gag peptide 45
SEGATPQDLNTMLNT

SEQ ID NO: 171 - protein, 15 aa, Artificial
  HIV-1 gag peptide 46
TPQDLNTMLNTVGGH

SEQ ID NO: 172 - protein, 15 aa, Artificial
  HIV-1 gag peptide 47
LNTMLNTVGGHQAAM

SEQ ID NO: 173 - protein, 15 aa, Artificial
  HIV-1 gag peptide 48
LNTVGGHQAAMQMLK

SEQ ID NO: 174 - protein, 15 aa, Artificial
  HIV-1 gag peptide 49
GGHQAAMQMLKETIN

SEQ ID NO: 175 - protein, 15 aa, Artificial
  HIV-1 gag peptide 50
AAMQMLKETINEEAA

SEQ ID NO: 176 - protein, 15 aa, Artificial
  HIV-1 gag peptide 51
QMLKETINEEAAEWD

SEQ ID NO: 177 - protein, 15 aa, Artificial
  HIV-1 gag peptide 52
ETINEEAAEWDRLHP

SEQ ID NO: 178 - protein, 15 aa, Artificial
  HIV-1 gag peptide 53
EEAAEWDRLHPVHAG

SEQ ID NO: 179 - protein, 15 aa, Artificial
  HIV-1 gag peptide 54
EWDRLHPVHAGPIAP

SEQ ID NO: 180 - protein, 15 aa, Artificial
  HIV-1 gag peptide 55
LHPVHAGPIAPGQMR

SEQ ID NO: 181 - protein, 15 aa, Artificial
  HIV-1 gag peptide 56
HAGPIAPGQMREPRG

SEQ ID NO: 182 - protein, 15 aa, Artificial
  HIV-1 gag peptide 57
IAPGQMREPRGSDIA

SEQ ID NO: 183 - protein, 15 aa, Artificial
  HIV-1 gag peptide 58
QMREPRGSDIAGTTS

SEQ ID NO: 184 - protein, 15 aa, Artificial
  HIV-1 gag peptide 59
PRGSDIAGTTSTLQE

SEQ ID NO: 185 - protein, 15 aa, Artificial
  HIV-1 gag peptide 60
DIAGTTSTLQEQIGW

SEQ ID NO: 186 - protein, 15 aa, Artificial
  HIV-1 gag peptide 61
TTSTLQEQIGWMTNN

SEQ ID NO: 187 - protein, 15 aa, Artificial
  HIV-1 gag peptide 62
LQEQIGWMTNNPPIP

SEQ ID NO: 188 - protein, 15 aa, Artificial
  HIV-1 gag peptide 63
IGWMTNNPPIPVGEI

SEQ ID NO: 189 - protein, 15 aa, Artificial
  HIV-1 gag peptide 64
TNNPPIPVGEIYKRW

SEQ ID NO: 190 - protein, 15 aa, Artificial
  HIV-1 gag peptide 65
PIPVGEIYKRWIILG

SEQ ID NO: 191 - protein, 15 aa, Artificial
  HIV-1 gag peptide 66
GEIYKRWIILGLNKI

SEQ ID NO: 192 - protein, 15 aa, Artificial
  HIV-1 gag peptide 67
KRWIILGLNKIVRMY

SEQ ID NO: 193 - protein, 15 aa, Artificial
  HIV-1 gag peptide 68
ILGLNKIVRMYSPTS

SEQ ID NO: 194 - protein, 15 aa, Artificial
  HIV-1 gag peptide 69
NKIVRMYSPTSILDI

SEQ ID NO: 195 - protein, 15 aa, Artificial
  HIV-1 gag peptide 70
RMYSPTSILDIRQGP

SEQ ID NO: 196 - protein, 15 aa, Artificial
  HIV-1 gag peptide 71
PTSILDIRQGPKEPF

SEQ ID NO: 197 - protein, 15 aa, Artificial
  HIV-1 gag peptide 72
LDIRQGPKEPFRDYV

SEQ ID NO: 198 - protein, 15 aa, Artificial
  HIV-1 gag peptide 73
QGPKEPFRDYVDRFY

SEQ ID NO: 199 - protein, 15 aa, Artificial
  HIV-1 gag peptide 74
EPFRDYVDRFYKTLR

SEQ ID NO: 200 - protein, 15 aa, Artificial
  HIV-1 gag peptide 75
DYVDRFYKTLRAEQA

SEQ ID NO: 201 - protein, 15 aa, Artificial
  HIV-1 gag peptide 76
RFYKTLRAEQASQEV

SEQ ID NO: 202 - protein, 15 aa, Artificial
  HIV-1 gag peptide 77
TLRAEQASQEVKNWM

SEQ ID NO: 203 - protein, 15 aa, Artificial
  HIV-1 gag peptide 78
EQASQEVKNWMTETL

SEQ ID NO: 204 - protein, 15 aa, Artificial
  HIV-1 gag peptide 79
QEVKNWMTETLLVQN

SEQ ID NO: 205 - protein, 15 aa, Artificial
  HIV-1 gag peptide 80
NWMTETLLVQNANPD

SEQ ID NO: 206 - protein, 15 aa, Artificial
  HIV-1 gag peptide 81
ETLLVQNANPDCKTI

SEQ ID NO: 207 - protein, 15 aa, Artificial
  HIV-1 gag peptide 82
VQNANPDCKTILKAL

SEQ ID NO: 208 - protein, 15 aa, Artificial
  HIV-1 gag peptide 83
NPDCKTILKALGPAA

SEQ ID NO: 209 - protein, 15 aa, Artificial
  HIV-1 gag peptide 84
KTILKALGPAATLEE

SEQ ID NO: 210 - protein, 15 aa, Artificial
  HIV-1 gag peptide 85
KALGPAATLEEMMTA

SEQ ID NO: 211 - protein, 15 aa, Artificial
  HIV-1 gag peptide 86
PAATLEEMMTACQGV

SEQ ID NO: 212 - protein, 15 aa, Artificial
  HIV-1 gag peptide 87
LEEMMTACQGVGGPG

SEQ ID NO: 213 - protein, 15 aa, Artificial
  HIV-1 gag peptide 88
MTACQGVGGPGHKAR

SEQ ID NO: 214 - protein, 15 aa, Artificial
  HIV-1 gag peptide 89
QGVGGPGHKARVLAE

SEQ ID NO: 215 - protein, 15 aa, Artificial
  HIV-1 gag peptide 90
GPGHKARVLAEAMSQ

SEQ ID NO: 216 - protein, 15 aa, Artificial
  HIV-1 gag peptide 91
KARVLAEAMSQVTNS

SEQ ID NO: 217 - protein, 15 aa, Artificial
  HIV-1 gag peptide 92
LAEAMSQVTNSATIM

SEQ ID NO: 218 - protein, 15 aa, Artificial
  HIV-1 gag peptide 93
MSQVTNSATIMMQRG

SEQ ID NO: 219 - protein, 15 aa, Artificial
  HIV-1 gag peptide 94
TNSATIMMQRGNFRN

SEQ ID NO: 220 - protein, 15 aa, Artificial
  HIV-1 gag peptide 95
TIMMQRGNFRNQRKT

SEQ ID NO: 221 - protein, 15 aa, Artificial
  HIV-1 gag peptide 96
QRGNFRNQRKTVKCF

SEQ ID NO: 222 - protein, 15 aa, Artificial
  HIV-1 gag peptide 97
FRNQRKTVKCFNCGK

SEQ ID NO: 223 - protein, 15 aa, Artificial
  HIV-1 gag peptide 98
RKTVKCFNCGKEGHI

SEQ ID NO: 224 - protein, 15 aa, Artificial
  HIV-1 gag peptide 99
VKCFNCGKEGHIAKN

SEQ ID NO: 225 - protein, 15 aa, Artificial
  HIV-1 gag peptide 100
NCGKEGHIAKNCRAP

SEQ ID NO: 226 - protein, 15 aa, Artificial
  HIV-1 gag peptide 101
EGHIAKNCRAPRKKG

SEQ ID NO: 227 - protein, 15 aa, Artificial
  HIV-1 gag peptide 102
AKNCRAPRKKGCWKC

SEQ ID NO: 228 - protein, 15 aa, Artificial
  HIV-1 gag peptide 103
RAPRKKGCWKCGKEG

SEQ ID NO: 229 - protein, 15 aa, Artificial
  HIV-1 gag peptide 104
KKGCWKCGKEGHQMK

SEQ ID NO: 230 - protein, 15 aa, Artificial
  HIV-1 gag peptide 105
WKCGKEGHQMKDCTE

SEQ ID NO: 231 - protein, 15 aa, Artificial
  HIV-1 gag peptide 106
KEGHQMKDCTERQAN

SEQ ID NO: 232 - protein, 15 aa, Artificial
  HIV-1 gag peptide 107
QMKDCTERQANFLGK

SEQ ID NO: 233 - protein, 15 aa, Artificial
  HIV-1 gag peptide 108
CTERQANFLGKIWPS

SEQ ID NO: 234 - protein, 15 aa, Artificial
  HIV-1 gag peptide 109
QANFLGKIWPSHKGR

SEQ ID NO: 235 - protein, 15 aa, Artificial
  HIV-1 gag peptide 110
LGKIWPSHKGRPGNF

SEQ ID NO: 236 - protein, 15 aa, Artificial
  HIV-1 gag peptide 111
WPSHKGRPGNFLQSR

SEQ ID NO: 237 - protein, 15 aa, Artificial
  HIV-1 gag peptide 112
KGRPGNFLQSRPEPT

SEQ ID NO: 238 - protein, 15 aa, Artificial
  HIV-1 gag peptide 113
GNFLQSRPEPTAPPE

SEQ ID NO: 239 - protein, 15 aa, Artificial
  HIV-1 gag peptide 114
QSRPEPTAPPEESFR

SEQ ID NO: 240 - protein, 15 aa, Artificial
  HIV-1 gag peptide 115
EPTAPPEESFRFGEE

SEQ ID NO: 241 - protein, 15 aa, Artificial
  HIV-1 gag peptide 116
PPEESFRFGEETTTP

SEQ ID NO: 242 - protein, 15 aa, Artificial
  HIV-1 gag peptide 117
SFRFGEETTTPSQKQ

SEQ ID NO: 243 - protein, 15 aa, Artificial
  HIV-1 gag peptide 118
GEETTTPSQKQEPID

SEQ ID NO: 244 - protein, 15 aa, Artificial
  HIV-1 gag peptide 119
TTTPSQKQEPIDKEL

SEQ ID NO: 245 - protein, 15 aa, Artificial
  HIV-1 gag peptide 120
SQKQEPIDKELYPLA

SEQ ID NO: 246 - protein, 15 aa, Artificial
  HIV-1 gag peptide 121
EPIDKELYPLASLRS

SEQ ID NO: 247 - protein, 15 aa, Artificial
  HIV-1 gag peptide 122
KELYPLASLRSLFGN

SEQ ID NO: 248 - protein, 15 aa, Artificial
  HIV-1 gag peptide 123
PLASLRSLFGNDPSS

SEQ ID NO: 249 - protein, 12 aa, Artificial
  HIV-1 gag peptide 124
LRSLFGNDPSSQ

SEQ ID NO: 250 - protein, 543 aa, Simian immunodeficiency virus
MGVRNSVLSGKKADELEKIRLRPNGKKKYMLKHVVWAANELDRFGLAESLLENKEGCQKILSVLAPLVPTGSENLKSLYNTVCVIWCIHAEEKVKHTEEAKQIVQRHLVVETGTTETMPKTSRPTAPSSGRGGNYPVQQIGGNYVHLPLSPRTLNAWVKLIEEKKFGAEVVIEEKKFGAEVVPGFQALSEGCTPYDINQMLNCVGDHQAAMQIIRDIINEEAADWDLQHPQPAPQQGQLREPSGSDIAGTTSSVDEQIQWMYRQQNPIPVGNIYRRWIQLGLQKCVRMYNPTNILDVKQGPKEPFQSYVDRFYKSLRAEQTDAAVKNWMTQTLLIQNANPDCTLLIQNANPDCKLVLKGLGVNPTLEEMLTACQGVGGPGQKARLMAEALKEALAPVPIPFAAAQQRGPRKPIKCWNCGKEGHSARQCRAPRRQGCWKCGKMDHVMAKCPDRQAGFLGLGPWGKKPRNFPMAQVHQGLMPTAPPEDPAVDLLKNYMQLGKQQREKQRESREKPREKQRESREKPYKEVTEDLLHLNSLFGGDQ

SEQ ID NO: 251 - protein, 533 aa, Human immunodeficiency virus type 1
MGARASVLSGGELDRWEKIRLRPGGKKKYKLKHIVWASRELERFAVNPGLLETSEGCRQILGQLQPSLQTGSEELRSLYNTVATLYCVHQRIEVKDTKEALEKIEEEQNKSKKKAQQAAADTGNSSQVSQNYPIVQNLQGQMVHQAISPRTLNAWVKVVEEKAFSPEVIPEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDETLLVQNANPDCKTILKALGPAATLEEMMTACQGVGGPGHKARVLAEAMSQVTNSATIMMQRGNFRNQRKTVKCFNCGKEGHIAKNCRAPRKKGCWKCGKEGHQMKDCTERQANFLGKIWPSHKGRPGNFLQSRPEPTAPPEESFRFGEETTTPSQKQEPIDKELYPLAEPIDKELYPLASLRSLFGNDPSSQ

(19) United States
(12) Patent Application Publication
(10) Pub. No.: US 2011/0008417 A1
(43) Pub. Date: Jan. 13, 2011

(54) IMMUNOMODULATING COMPOSITIONS AND USES THEREFOR

(75) Inventors: Vivienne Peut, Victoria (AU); Robert De Rose, Victoria (AU); Stephen Kent, Victoria (AU)

Correspondence Address: KNOBBE MARTENS OLSON & BEAR LLP, 2040 MAIN STREET, FOURTEENTH FLOOR, IRVINE, CA, 92614, US

(73) Assignee: OPAL THERAPEUTICS PTY LTD, Melbourne, Victoria (AU)

(21) Appl. No.: 12/811,304

(22) PCT Filed: Jan. 16, 2008
(86) PCT No.: PCT/AU08/00056

§ 371 Date: Sep. 28, 2010

(51) Int. Cl. A61K 9/127; A61K 35/12; A61K 39/00; A61K 9/00; A61K 9/50; C12N 5/078; C12N 5/0783; C12N 5/0784; C12N 5/0786; C12N 5/0781; A61P 37/04; A61P 31/12

(52) U.S. Cl. 424/450; 424/93.71; 424/184.1; 424/490; 424/498; 435/325

(57) ABSTRACT

This invention discloses compositions that consist essentially of a Gag polypeptide or at least one portion thereof, and optionally antigen-presenting cells or their precursors, for treating or preventing lentiviral infections including the treatment or prevention of related acquired immunodeficiency diseases. In certain embodiments, the compositions consist essentially of a plurality of overlapping and/or non-overlapping peptides derived from a single Gag polypeptide or from different Gag polypeptides.

### FIELD OF THE INVENTION

This invention relates generally to modulation of immune responses. More particularly, the present invention relates to compositions that consist essentially of a Gag polypeptide or at least one portion thereof, and optionally antigen-presenting cells or their precursors, for treating or preventing lentiviral infections including the treatment or prevention of related acquired immunodeficiency diseases. In certain embodiments, the compositions consist essentially of a plurality of overlapping and/or non-overlapping peptides derived from a single Gag polypeptide or from different Gag polypeptides.

Bibliographic details of various publications numerically referred to in this specification are listed at the end of the description.

### BACKGROUND OF THE INVENTION

Effective immunotherapies for human immunodeficiency virus (HIV) are needed. Drug therapies are life-long with significant toxicities. Several attempts at immunotherapy of HIV using conventional vaccines have thus far been poorly immunogenic and weakly efficacious in human trials¹⁻⁴. The use of professional antigen-presenting cells such as dendritic cells to deliver HIV immunotherapies has shown efficacy in macaques and pilot humans studies but is limited to highly specialized facilities^{5, 6}. A simple intermittent immunotherapy that reduces the need for long-term antiretroviral therapy (ART) would be a quantum advance in treating HIV.

Recently, an immunotherapy was developed by the present inventors, which involves treating unfractionated whole blood or peripheral blood mononuclear cells (PBMC) with overlapping virus-derived peptides. Significantly, this simple immunotherapy, termed OPAL (Overlapping Peptide-pulsed Autologous Leukocytes) produced robust cell mediated immune responses against viral infections, including HIV infections, in outbred populations^{7, 8}. The OPAL technology has several advantages including (1) no requirement for prolonged ex vivo culture of antigen-presenting cells, (2) induction of CD4⁺ and CD8⁺ T-cell responses to both structural and regulatory proteins, and (3) facile production of peptide antigens.

The present inventors have now discovered that when pigtail macaques are immunized with fresh blood cells exposed to overlapping simian immunodeficiency virus (SIV) peptides, there is no difference in viral outcome between animals immunized against Gag alone (“OPAL-Gag animals”) and ones immunized against the entire SIV proteome (“OPAL-All animals”), suggesting that Gag alone is an effective antigen for T-cell immunotherapies. Additionally, it was found unexpectedly that Gag-specific CD4⁺ and CD8⁺ T-cell responses in OPAL-Gag animals were significantly greater than those in the OPAL-All animals, despite an identical dose of Gag overlapping peptides. This suggests that there is antigenic competition between peptides from Gag and the other SIV proteins and that inducing immunodominant non-Gag T-cell responses by multi-protein HIV vaccines may limit the development of therapeutic or prophylactic Gag-specific T-cell responses. These discoveries have been reduced to practice in novel compositions and methods for treating or preventing lentiviral infections, including the treatment and prevention of diseases associated with those infections.

### SUMMARY OF THE INVENTION

Accordingly, one aspect of the present invention provides methods for treating or preventing a lentivirus infection in a subject, wherein the methods consist essentially of increasing in the subject the number of antigen-presenting cells or antigen-presenting cells precursors, which present on their surface at least one peptide that comprises an amino acid sequence corresponding to a portion of a Gag polypeptide. Such antigen-presenting cells and precursors are also referred to herein as “Gag-specific antigen-presenting cells” and “Gag-specific antigen-presenting cell precursors,” respectively. Non-limiting antigen presenting cells include dendritic cells, macrophages and Langerhans cells.

Any suitable method of increasing the number of Gag-specific antigen-presenting cells or precursors in the subject is contemplated by the present invention. In some embodiments, the subject is administered an immune stimulator that increases the number of antigen-presenting cells or antigen-presenting cells precursors, which present on their surface at least one peptide that comprises an amino acid sequence corresponding to a portion of a Gag polypeptide. In illustrative examples of this type, the immune stimulator is in the form of antigen-presenting cells or precursors, which have been contacted with a composition that consists essentially of a Gag polypeptide or at least one peptide that comprises an amino acid sequence corresponding to a Gag polypeptide for a time and under conditions sufficient for the Gag polypeptide or the peptide(s), or processed forms of the Gag polypeptide or the peptide(s), to be presented by the antigen-presenting cells or by their precursors. In other illustrative examples, the immune stimulator is in the form of antigen-presenting cells or antigen-presenting cell precursors, containing a nucleic acid construct that comprises a nucleotide sequence encoding a Gag polypeptide or at least one peptide that comprises an amino acid sequence corresponding to a portion of a Gag polypeptide, wherein the nucleotide sequence is operably connected to a regulatory element that is operable in the antigen-presenting cells or their precursors. Such nucleic acid constructs are also referred to herein as “Gag-expressing nucleic acid constructs”. In still other illustrative examples, the immune stimulator is in the form of a composition that consists essentially of at least one Gag molecule selected from a Gag polypeptide, a peptide that comprises a sequence corresponding to a portion of a Gag polypeptide, and a Gag-expressing nucleic acid construct. In illustrative examples of this type, a respective Gag molecule is in a form that is suitable for introduction (e.g., by transformation, internalization, endocytosis or phagocytosis) into the antigen-presenting cells or their precursors, which includes soluble and particulate forms of the Gag molecule. In some embodiments, the Gag molecule(s) is (are) contained or otherwise associated with a particle, illustrative examples of which include liposomes, micelles, lipidic particles, ceramic/inorganic particles and polymeric particles. In some embodiments, the methods exclude administering to the subject antigen-presenting cells that present on their surface peptides that comprise amino acid sequences corresponding to portions of other lentivirus polypeptides. In some embodiments, the methods exclude administering to the subject other lentivirus molecules or antigen-presenting cells that have been contacted with other lentivirus molecules, wherein the other lentivirus molecules are selected from non-Gag polypeptides of the lentivirus, portions of non-Gag polypeptides of the lentivirus and nucleic acid constructs from which the non-Gag polypeptides or the non-Gag polypeptide portions are expressible. In some embodiments, the compositions comprise a proteinaceous component that consists of at least one Gag molecule selected from a Gag polypeptide, a peptide that comprises a sequence corresponding to a portion of a Gag polypeptide and a Gag-expressing nucleic acid construct.

In certain embodiments, the immune stimulator is administered with a pharmaceutically acceptable carrier and/or diluent. Alternatively, or in addition, the immune stimulator is administered with an adjuvant or with a compound that stabilizes a Gag molecule as broadly described above against degradation by host enzymes. Suitably, the lentivirus is selected from human immunodeficiency virus (HIV) and simian immunodeficiency virus (SIV).

In some embodiments, the immune stimulator consists essentially of a plurality of peptides wherein individual peptides comprise different portions of an amino acid sequence corresponding to a Gag polypeptide and optionally display partial sequence identity or similarity to at least one other peptide of the plurality of peptides. In illustrative examples of this type, the partial sequence identity or similarity is contained at one or both ends of an individual peptide. Suitably, at one or both of these ends there are at least 4, 5, 6, 7, 8, 9, 10, 11, 12, 13, 14 contiguous amino acid residues whose sequence is identical or similar to an amino acid sequence contained within at least one other of the peptides. In certain embodiments, the peptide is at least 6, 7, 8, 9, 10, 11, 12, 13, 14, 15, 16, 17, 18, 19, 20, 21, 22, 23, 24, 25, 26, 27, 28, 29 or 30 amino acid residues in length and suitably no more than about 500, 200, 100, 80, 60, 50, 40 amino acid residues in length. Suitably, the length of the peptides is selected to enhance the production of a cytolytic T lymphocyte response (e.g., peptides of about 8 to about 10 amino acids in length), or a T helper lymphocyte response (e.g., peptides of about 12 to about 20 amino acids in length). In some embodiments, the peptide sequences are derived from at least about 30, 31, 32, 33, 34, 35, 36, 37, 38, 39, 40, 41, 42. 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% of the sequence corresponding to the Gag polypeptide. In specific embodiments, the plurality of peptides comprises peptides from two or more different Gag polypeptides.

Accordingly, in a related aspect, the present invention contemplates methods for treating or preventing a lentivirus infection in a subject, wherein the methods comprise increasing in the subject the number of Gag-specific antigen-presenting cells or Gag-specific antigen-presenting cell precursors, which present on their surface at least one peptide that comprises an amino acid sequence corresponding to a portion of a Gag polypeptide, wherein the Gag-specific antigen-presenting cells or the Gag-specific antigen-presenting cell precursors are produced by contacting antigen-presenting cells or antigen-presenting cell precursors with a composition that consists essentially of a plurality of peptides for a time and under conditions sufficient for the peptides, or processed forms of the peptides, to be presented by the antigen-presenting cells or by the precursors on their surface, wherein individual peptides of the composition comprise different portions of an amino acid sequence corresponding to a Gag polypeptide and optionally display partial sequence identity or similarity to at least one other peptide of the plurality of peptides. In some embodiments, the subject is administered the Gag-specific antigen-presenting cells or the Gag-specific antigen-presenting cell precursors. In other embodiments, the subject is administered the composition.

Another aspect of the present invention provides compositions for treating or preventing a lentivirus infection, wherein the compositions consist essentially of Gag-specific antigen-presenting cells or Gag-specific antigen-presenting cell precursors as broadly described above or of at least one Gag molecule selected from a Gag polypeptide, a peptide that comprises a sequence corresponding to a portion of a Gag polypeptide and a Gag-expressing nucleic acid construct, as broadly described above. In some embodiments, the or each Gag molecule is in particulate form.

In a related aspect, the invention provides processes for producing antigen-presenting cells for treating or preventing a lentivirus infection. These process generally comprise contacting antigen-presenting cells or antigen-presenting cell precursors with a composition that consists essentially of at least one Gag molecule selected from a Gag polypeptide, a peptide that comprises a sequence corresponding to a portion of a Gag polypeptide and a Gag-expressing nucleic acid construct, as broadly described above, for a time and under conditions sufficient for at least one peptide that comprises an amino acid sequence corresponding to a portion of a Gag polypeptide to be presented by the antigen-presenting cells or by their precursors on their surface. Suitably, when precursors are used, the precursors are cultured for a time and under conditions sufficient to differentiate antigen-presenting cells from the precursors.

In some embodiments, the or each Gag molecule is contacted with substantially purified population of antigen-presenting cells or their precursors. In other embodiments, individual Gag molecules are contacted with a heterogeneous population of antigen-presenting cells or their precursors. In these embodiments, the heterogeneous population of cells can be blood or peripheral blood mononuclear cells. Typically, the antigen-presenting cells or their precursors are selected from monocytes, macrophages, cells of myeloid lineage, B cells, dendritic cells or Langerhans cells. In still other embodiments, the Gag molecule(s) is (are) contacted with an uncultured population of antigen-presenting cells or their precursors. Accordingly, the uncultured population can be homogeneous or heterogeneous, illustrative examples of which include whole blood, fresh blood, or fractions thereof such as, but not limited to, peripheral blood mononuclear cells, buffy coat fractions of whole blood, packed red cells, irradiated blood, dendritic cells, monocytes, macrophages, neutrophils, lymphocytes, natural killer cells and natural killer T cells. In some embodiments, the uncultured population o, which is contacted with the Gag molecule(s), has not been subjected to activating conditions.

The Gag-specific antigen-presenting cells broadly described above are also useful for producing lymphocytes, including T lymphocytes and B lymphocytes, for modulating an immune response to a Gag polypeptide. Accordingly, in yet another aspect, the invention provides methods for producing Gag-primed lymphocytes, wherein the methods generally comprise contacting a population of lymphocytes, or their precursors, with a Gag-specific antigen-presenting cell as broadly described above for a time and under conditions sufficient to prime the lymphocytes to respond to the Gag polypeptide.

In yet another aspect, the present invention embraces methods for treating or preventing a lentivirus infection in a subject. These methods generally comprise administering to the subject an immune stimulator as broadly described above, or Gag-primed lymphocytes as broadly described above in an amount that is effective to treat or prevent the lentivirus infection. In some embodiments, the immune stimulator or Gag-primed lymphocytes are administered systemically, typically by injection.

In a related aspect, the invention provides methods for treating or preventing an acquired immunodeficiency disease in a subject. These methods generally comprise administering to the subject an immune stimulator as broadly described above, or Gag-primed lymphocytes as broadly described above in an amount that is effective to treat or prevent the disease.

In still another aspect, the invention contemplates the use of an immune stimulator as broadly described above, or Gag-primed lymphocytes as broadly described above, for treating or preventing a condition selected from a lentivirus infection and an acquired immunodeficiency disease. In some embodiments, the use comprises preparation of a medicament that is suitable for the treatment or prevention of that condition.

### BRIEF DESCRIPTION OF THE DRAWINGS

FIG. 1 is a graphical representation illustrating T-cell immunogenicity of OPAL vaccination. SIV Gag-specific CD4 (a) and CD8 (b) T-cells expressing IFN-γ were studied over time by intracellular cytokine staining. Mean±standard error of vaccine groups compared to control unvaccinated animals (circles) is shown. The primary OPAL vaccinations of macaques (arrows, weeks 4, 6, 8 and 10 after SIV_{mac251} infection) consisted of autologous PBMC pulsed with either overlapping SIV Gag 15mer peptides (OPAL-Gag, triangles) or peptides spanning all 9 SIV proteins (OPAL-All, squares). Initial vaccinations were given under the cover of antiretroviral treatment (ART). Animals were re-boosted with OPAL immunotherapy in the same randomised groups, without ART, at weeks 39, 42 and 42. At week 12, two weeks after the last vaccination, CD4 (c) and CD8 (d) T-cells to pools of overlapping peptides spanning SIV Gag, Env, Pol or combined Regulatory/Accessory proteins (Nef, Tat, Rev, Vif, Vpx, Vpr [Reg]) were assessed in all animals by intracellular cytokine staining. In addition, responses to a SIV Gag CD8 T-cell epitope KP9, were assessed by a Mane-A*10/KP9 tetramer. Mean±standard error of vaccine groups is shown along with 2-sided t-test p values of <0.10. (e) SIV Gag specific CD8 T-cell responses correlated inversely with CD8 T-cell responses to the summation of non-Gag (Env⁺Pol⁺Regulatory protein) responses across all 21 live OPAL-immunized animals. The animals with >50% CD8 T-cell responses to the combined pool had total responses of 50.4% and 54.5%, primarily to Env (50.1% and 54.2% respectively). Spearman rank correlation is shown.

FIG. 2 is a graphical representation showing efficacy of OPAL immunotherapy. Antiretroviral therapy (ART) was withdrawn at week 10, after the last vaccination, and (a) plasma SIV RNA followed. The 26 animals that controlled viremia on ART are illustrated with mean±standard error of vaccine groups. (b) Survival of the vaccinated and controls animals is shown.

FIG. 3 is a graphic representation illustrating non-Gag T cell immunogenicity of OPAL vaccination. SW-specific CD4⁺ and CD8⁺ T-cells expressing IFN-γ were studied over time by intracellular cytokine staining to Env (a, b), Pol (c, d) and a pool of overlapping peptides spanning combined Regulatory/Accessory proteins (RTNVVV, e, f). Mean±standard error of vaccine groups compared to control unvaccinated animals (circles) is shown. Four initial vaccinations were given weeks 4-10 and a second set of 3 immunizations given weeks 36-42.

FIG. 4 is a graphic representation showing a comparison between CD8⁺ T Cell Env Responders and Gag Responders. Six Env-only responders, 3 Gag-only responders, 3 animals with both Env- and Gag-specific CD8 T-cell responses and 7 unvaccinated controls were studied for. A. Viral load. B. Peripheral CD4 T-cell levels. C. Survival graph showing 2 of 6 Env-only responders euthanised by week 44, 0 of 3 Gag responders euthanised by week 64, 2 of 3 animals with both Env- and Gag-specific responses euthanised by week 44 and 7 of 11 control animals euthanised by week 64 post infection. No ManeA*10 positive animals included. A last observation carried forward analysis was used for VL and CD4⁺ T cell counts where animals were euthanised prior to week 64.

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

The term “activating conditions” refers to treatment conditions that lead to the expression of each of CD2, CD83, CD14, MHC class I, MHC class II and TNF-α at a level or functional activity that results from an activating treatment condition selected from: incubating the antigen-presenting cells or their precursors in the presence of an agent selected from cytokines (e.g., IL-4, GM-CSF or a type I interferon), chemokines, mitogens, lipopolysaccharide, or agents that induce interferon synthesis in the antigen-presenting cells or their precursors; or exposing the antigen-presenting cells or their precursors to physical stress. However, it shall be understood that the term “activating conditions” excludes treatment conditions that result in negligible activation of the cells, e.g., when less than about 20%, 15%, 10%, 9%, 8%, 7%, 6%, 5%, 4%, 3%, 2%, 1%, 0.5%, 0.2% or 0.1% of the cells are activated, or when each of CD2, CD83, CD14, MHC class I, MHC class II and TNF-α is expressed at a level or functional activity that is up to about 10% ( 1/10), 20% (⅕), 30% ( 3/10) 40% (⅖), 50% (½), 60% (⅗), 70% ( 7/10), 80% (⅘) or 90% ( 9/10) of its level or functional activity in antigen-presenting cells or their precursors subjected to an activating treatment condition mentioned above.

By “antigen” is meant all, or part of, a protein, peptide, or other molecule or macromolecule capable of eliciting an immune response in a vertebrate animal, preferably a mammal. Such antigens are also reactive with antibodies from animals immunised with said protein, peptide, or other molecule or macromolecule.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity.

By “autologous” is meant something (e.g., cells, tissues etc) derived from the same organism.

The term “allogeneic” as used herein refers to cells, tissues, organisms etc that are of different genetic constitution.

By “alloantigen” is meant an antigen found only in some members of a species, such as blood group antigens. By contrast a “xenoantigen” refers to an antigen that is present in members of one species but not members of another. Correspondingly, an “allograft” is a graft between members of the same species and a “xenograft” is a graft between members of a different species.

Throughout this specification, unless the context requires otherwise, the words “comprise,” “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, the terms “culturing,” “culture” and the like refer to the set of procedures used in vitro where a population of cells (or a single cell) is incubated under conditions which have been shown to support the growth or maintenance of the cells in vitro. The art recognises a wide number of formats, media, temperature ranges, gas concentrations etc. which need to be defined in a culture system. The parameters will vary based on the format selected and the specific needs of the individual who practices the methods herein disclosed. However, it is recognised that the determination of culture parameters is routine in nature.

By “corresponds to” or “corresponding to” is meant an antigen which encodes an amino acid sequence that displays substantial similarity to an amino acid sequence in a target antigen. In general the antigen will display at least about 30, 31, 32, 33, 34, 35, 36, 37, 38, 39, 40, 41, 42. 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% similarity or identity to at least a portion of the target antigen.

By “effective amount,” in the context of modulating an immune response or treating or preventing a disease or condition, is meant the administration of that amount of composition to an individual in need thereof, either in a single dose or as part of a series, that is effective for that modulation, treatment or prevention. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

By “expression vector” is meant any autonomous genetic element capable of directing the synthesis of a protein encoded by the vector. Such expression vectors are known by practitioners in the art.

The term “gene” as used herein refers to any and all discrete coding regions of the cell's genome, as well as associated non-coding and regulatory regions. The gene is also intended to mean the open reading frame encoding specific polypeptides, introns, and adjacent 5′ and 3′ non-coding nucleotide sequences involved in the regulation of expression. In this regard, the gene may further comprise control signals such as promoters, enhancers, termination and/or polyadenylation signals that are naturally associated with a given gene, or heterologous control signals. The DNA sequences may be cDNA or genomic DNA or a fragment thereof. The gene may be introduced into an appropriate vector for extrachromosomal maintenance or for integration into the host.

A compound or composition is “immunogenic” if it is capable of either: a) generating an immune response against an antigen (e.g., a viral antigen) in a naive individual; or b) reconstituting, boosting, or maintaining an immune response in an individual beyond what would occur if the compound or composition was not administered. A compound or composition is immunogenic if it is capable of attaining either of these criteria when administered in single or multiple doses.

Reference herein to “immuno-interactive” includes reference to any interaction, reaction, or other form of association between molecules and in particular where one of the molecules is, or mimics, a component of the immune system.

By “isolated” is meant material that is substantially or essentially free from components that normally accompany it in its native state.

The term “lentiviruses” includes and encompasses: primate lentiviruses, e.g., human immunodeficiency virus types 1 and 2 (HIV-1/HIV-2); simian immunodeficiency virus (SIV) from Chimpanzee (SIV_{cpz}), Sooty mangabey (SIVₛₘₘ), African Green Monkey (SIV_{agm}), Syke's monkey (SIV_{syk}), Mandrill (SIV_{mnd}) and Macaque (SIV_{mac}). Lentiviruses also include feline lentiviruses, e.g., Feline immunodeficiency virus (FIV); Bovine lentiviruses, e.g., Bovine immunodeficiency virus (BIV); Ovine lentiviruses, e.g., Maedi/Visna virus (MVV) and Caprine arthritis encephalitis virus (CAEV); and Equine lentiviruses, e.g., Equine infectious anemia virus (EIAV). All lentiviruses express at least two additional regulatory proteins (Tat, Rev) in addition to Gag, Pol, and Env proteins. Primate lentiviruses produce other accessory proteins including Nef, Vpr, Vpu, Vpx, and Vif. Generally, lentiviruses are the causative agents of a variety of disease, including, in addition to immunodeficiency, neurological degeneration, and arthritis. Nucleotide sequences of the various lentiviruses can be found in GenBank under the following accession Nos. (from J. M. Coffin, S. H. Hughes, and H. E. Varmus, “Retroviruses” Cold Spring Harbor Laboratory Press, 199,7 p 804): 1) HIV-1: K03455, M19921, K02013, M38431, M38429, K02007 and M17449; 2) HIV-2: M30502, J04542, M30895, J04498, M15390, M31113 and L07625; 3) SIV: M29975, M30931, M58410, M66437, L06042, M33262, M19499, M32741, M31345 and L03295; 4) FIV: M25381, M36968 and U11820; 5) BIV. M32690; 6) EIAV: M16575, M87581 and U01866; 6) Visna: M10608, M51543, L06906, M60609 and M60610; 7) CAEV: M33677; and 8) Ovine lentivirus M31646 and M34193. Amino acid sequences for the various lentiviral polypeptides are also provided in these GenBank accessions. Lentiviral DNA can also be obtained from the American Type Culture Collection (ATCC). For example, feline immunodeficiency virus is available under ATCC Designation No. VR-2333 and VR-3112. Equine infectious anemia virus A is available under ATCC Designation No. VR-778. Caprine arthritis-encephalitis virus is available under ATCC Designation No. VR-905. Visna virus is available under ATCC Designation No. VR-779.

By “modulating” is meant increasing or decreasing, either directly or indirectly, the immune response of an individual. In certain embodiments, “modulation” or “modulating” means that a desired/selected response is more efficient (e.g., at least 10%, 20%, 30%, 40%, 50%, 60% or more), more rapid (e.g., at least 10%, 20%, 30%, 40%, 50%, 60% or more), greater in magnitude (e.g., at least 10%, 20%, 30%, 40%, 50%, 60% or more), and/or more easily induced (e.g., at least 10%, 20%, 30%, 40%, 50%, 60% or more) than in the absence of an antigen or than if the antigen had been used alone.

The term “operably connected” or “operably linked” as used herein means placing a structural gene under the regulatory control of a regulatory element including but not limited to a promoter, which then controls the transcription and optionally translation of the gene. In the construction of heterologous promoter/structural gene combinations, it is generally preferred to position the genetic sequence or promoter at a distance from the gene transcription start site that is approximately the same as the distance between that genetic sequence or promoter and the gene it controls in its natural setting; i.e. the gene from which the genetic sequence or promoter is derived. As is known in the art, some variation in this distance can be accommodated without loss of function. Similarly, the preferred positioning of a regulatory sequence element with respect to a heterologous gene to be placed under its control is defined by the positioning of the element in its natural setting; i.e. the genes from which it is derived.

The terms “patient,” “subject,” “host” or “individual” used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, any member of the subphylum Chordata including primates, rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc), marine mammals (e.g., dolphins, whales), reptiles (snakes, frogs, lizards etc), and fish. A preferred subject is a primate (e.g., a human, monkey, chimpanzee) in need of treatment or prophylaxis for a condition or disease. However, it will be understood that the aforementioned terms do not imply that symptoms are present.

By “pharmaceutically-acceptable carrier” is meant a solid or liquid filler, diluent or encapsulating substance that may be safely used in topical or systemic administration.

“Polypeptide,” “peptide” and “protein” are used interchangeably herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers.

Reference herein to a “promoter” is to be taken in its broadest context and includes the transcriptional regulatory sequences of a classical genomic gene, including the TATA box which is required for accurate transcription initiation, with or without a CCAAT box sequence and additional regulatory elements (i.e. upstream activating sequences, enhancers and silencers) which alter gene expression in response to developmental and/or environmental stimuli, or in a tissue-specific or cell-type-specific manner. A promoter is usually, but not necessarily, positioned upstream or 5′, of a structural gene, the expression of which it regulates. Furthermore, the regulatory elements comprising a promoter are usually positioned within 2 kb of the start site of transcription of the gene. Preferred promoters according to the invention may contain additional copies of one or more specific regulatory elements to further enhance expression in a cell, and/or to alter the timing of expression of a structural gene to which it is operably connected.

The terms “purified polypeptide” or “purified peptide” mean that the polypeptide or peptide is substantially free of cellular material or other contaminating proteins from the cell or tissue source from which the polypeptide or peptide is derived, or substantially free from chemical precursors or other chemicals when chemically synthesized. “Substantially free” means that a preparation of a Gag polypeptide or peptide of the invention is at least 10% pure. In certain embodiments, the preparation of Gag polypeptide or peptide has less than about 30%, 25%, 20%, 15%, 10% and desirably 5% (by dry weight), of non-peptide protein (also referred to herein as a “contaminating protein”), or of chemical precursors or non-peptide chemicals. The invention includes isolated or purified preparations of at least 0.01, 0.1, 1.0, and 10 milligrams in dry weight.

The term “recombinant polynucleotide” as used herein refers to a polynucleotide formed in vitro by the manipulation of nucleic acid into a form not normally found in nature. For example, the recombinant polynucleotide may be in the form of an expression vector. Generally, such expression vectors include transcriptional and translational regulatory nucleic acid operably linked to the nucleotide sequence.

By “recombinant polypeptide” is meant a polypeptide made using recombinant techniques, i.e., through the expression of a recombinant polynucleotide.

By “regulatory element” or “regulatory sequence” is meant nucleic acid sequences (e.g., DNA) necessary for expression of an operably linked coding sequence in a particular host cell. The regulatory sequences that are suitable for prokaryotic cells for example, include a promoter, and optionally a cis-acting sequence such as an operator sequence and a ribosome binding site. Control sequences that are suitable for eukaryotic cells include promoters, polyadenylation signals, transcriptional enhancers, translational enhancers, leader or trailing sequences that modulate mRNA stability, as well as targeting sequences that target a product encoded by a transcribed polynucleotide to an intracellular compartment within a cell or to the extracellular environment.

The terms “sequence identity” and “identity” are used interchangeably herein to refer to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software.

The terms “sequence similarity” and “similarity” are used interchangeably herein to refer to the percentage number of amino acids that are identical or constitute conservative substitutions as defined in Table 1 infra. Similarity may be determined using sequence comparison programs such as GAP (Deveraux et al. 1984, Nucleic Acids Research 12, 387-395). In this way, sequences of a similar or substantially different length to those cited herein might be compared by insertion of gaps into the alignment, such gaps being determined, for example, by the comparison algorithm used by GAP.

Terms used to describe sequence relationships between two or more polynucleotides or polypeptides include “reference sequence,” “comparison window,” “sequence identity,” “percentage of sequence identity” and “substantial identity”. A “reference sequence” is at least 12 but frequently 15 to 18 and often at least 25 monomer units, inclusive of nucleotides and amino acid residues, in length. Because two polypeptides may each comprise (1) a sequence (i.e., only a portion of the complete polypeptide sequence) that is similar between the two polypeptides, and (2) a sequence that is divergent between the two polypeptides, sequence comparisons between two (or more) polypeptides are typically performed by comparing sequences of the two polypeptides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

By “substantially purified population” and the like is meant that greater than about 80%, usually greater than about 90%, more usually greater than about 95%, typically greater than about 98%, and more typically greater than about 99% of the cells in the population are antigen-presenting cells of a chosen type.

By “treatment” is meant at least an amelioration of the symptoms associated with the pathological condition afflicting the host, where amelioration is used in a broad sense to refer to at least a reduction in the magnitude of a parameter, e.g., symptom, associated with the pathological condition being treated, such as the number of viral particles per unit blood. As such, treatment also includes situations where the pathological condition, or at least symptoms associated therewith, are completely inhibited, e.g., prevented from happening, or stopped, e.g., terminated, such that the host no longer suffers from the pathological condition, or at least the symptoms that characterize the pathological condition.

The term “uncultured” as used herein refers to a population of cells (or a single cell), which have been removed from an animal and incubated or processed under conditions that do not result in the growth or expansion of the cells in vitro, or that result in negligible growth or expansion of the cells (e.g., an increase of less than about 50%, 40%, 30%, 20%, 15%, 10%, 9%, 8%, 7%, 6%, 5%, 4%, 3%, 2%, 1%, 0.5%, 0.2% or 0.1% in cell number as compared to the number of cells at the commencement of the incubation or processing). In certain desirable embodiments, the population of cells (or the single cell) is incubated or processed under conditions supporting the maintenance of the cells in vitro.

By “vector” is meant a nucleic acid molecule, suitably a DNA molecule derived, for example, from a plasmid, bacteriophage, or plant virus, into which a nucleic acid sequence may be inserted or cloned. A vector preferably contains one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector may also include a selection marker such as an antibiotic resistance gene that can be used for selection of suitable transformants.

### 2. Abbreviations

The following abbreviations are used throughout the application:

AIDS=acquired immunodeficiency disease

APC=antigen-presenting cell

ART=antiretroviral therapy

BIV=bovine immunodeficiency virus

CAEV=caprine arthritis encephalitis virus

cm=centimeters

CTL=cytotoxic T lymphocyte

EIAV=equine infectious anemia virus

FIV=feline immunodeficiency virus

g=grams

G-CSF=granulocyte colony stimulating factor

GM-CSF=granulocyte macrophage colony stimulating factor

hr=hour

HIV=human immunodeficiency virus

IFN-γ=interferon gamma

IFN-α=interferon alpha

IL=interleukin

IV=intravenous

mAb=monoclonal antibody

mL=milliliters

mg=milligrams

μg=micrograms

μL=microliters

μm=micrometers

MVV=Maedi/Visna virus

nm=nanometers

NF-κB=nuclear factor kappa B

OPAL=overlapping peptide-pulsed autologous leukocytes

PMBC=peripheral blood mononuclear cells

pro-GP=progenipoietin

SIV=simian immunodeficiency virus

TGFβ=transforming growth factor beta

TNF=Tumor necrosis factor

VL=viral load

VLP=virus-like particles

### 3. Immunomodulating Compositions Based on Lentiviral Gag Antigen

The present invention is predicated in part on the surprising discovery that there is no difference in viral outcome between animals immunized against SIV Gag alone and animals immunized against the entire SIV proteome. Additionally, it has been found unexpectedly that immunizing against SIV Gag as well as other SIV antigens induces immunodominant non-Gag T-cell responses, which may limit the development of therapeutic or prophylactic Gag-specific T-cell responses. Based on these observations, the inventors propose that lentiviral therapy or prophylaxis in a subject does not need to aim for maximally broad multi-protein lentiviral vaccines but is instead achievable essentially by increasing the number of antigen-presenting cells or antigen-presenting cell precursors (also referred herein, respectively as Gag-specific antigen-presenting cells or Gag-specific antigen-presenting cell precursors) in the subject, which present at least one peptide that comprises an amino acid sequence corresponding to a portion of a Gag polypeptide. The present invention thus provides methods of treating or preventing a lentivirus infection in a subject, wherein the methods consist essentially of increasing the number of Gag-specific antigen-presenting cells or precursors in the subject.

In some embodiments, the methods comprise administering to the subject an effective amount of an immune stimulator that increases the number of Gag-specific antigen-presenting cells or precursors thereof. For example, the immune stimulator may consist essentially of a Gag polypeptide or at least one peptide (also referred to herein as a Gag peptide) that comprises an amino acid sequence that corresponds to a portion of a Gag polypeptide. Alternatively, the immune stimulator may consist essentially of a nucleic acid construct that comprises a coding sequence for a Gag polypeptide or at least one Gag peptide, operably linked to a regulatory sequence. In other illustrative examples, the immune stimulator consists essentially of autologous or allogeneic Gag-specific antigen-presenting cells or their precursors. Non-limiting antigen presenting cells include dendritic cells, macrophages and Langerhans cells.

In illustrative examples, therefore, the number of Gag-specific antigen-presenting cells or Gag-specific antigen-presenting cell precursors can be increased by:

(1) administering to the subject antigen-presenting cells or precursors (e.g., autologous antigen-presenting cells or precursors from the subject or allogeneic antigen-presenting cells or precursors from a histocompatible donor), which have been contacted (e.g., ex vivo or in vivo) with a composition that consists essentially of a Gag polypeptide or at least one peptide that comprises an amino acid sequence corresponding to a Gag polypeptide for a time and under conditions sufficient for the Gag polypeptide or the peptide(s), or processed forms of the Gag polypeptide or the peptide(s), to be presented by the antigen-presenting cells or by their precursors;

(2) administering to the subject antigen-presenting cells or precursors (e.g., autologous antigen-presenting cells or precursors from the subject or allogeneic antigen-presenting cells or precursors from a histocompatible donor), which contain a nucleic acid construct (also referred to herein as a Gag-expressing nucleic acid construct) that comprises a nucleotide sequence encoding a Gag polypeptide or at least one peptide that comprises an amino acid sequence corresponding to a portion of a Gag polypeptide, wherein the nucleotide sequence is operably connected to a promoter that is operable in the antigen-presenting cells or their precursors;

(3) administering to the subject a composition that consists essentially of at least one Gag molecule selected from a Gag polypeptide, a peptide that comprises a sequence corresponding to a portion of a Gag polypeptide, and a Gag-expressing nucleic acid construct. The Gag molecule may be in soluble or particulate form.

### 3.1 Gag Polypeptides and Peptides

The present invention contemplates the use of full-length Gag polypeptides as well as peptides (also referred to herein as Gag peptides) which comprise amino acid sequences corresponding to portions of full-length Gag polypeptides, for producing Gag-specific antigen-presenting cell or precursors. Illustrative peptides comprise at least about 5, 6, 7, 8, 9, 10, 11, 12, 13, 14, 15, 16, 17, 18, 19, 20, 21, 22, 23, 24, 25, 30, 40, 50, 60, 70, 80, 90, 100, 120, 150, 300, 400 or 500 contiguous amino acid residues, or almost up to the total number of amino acids present in a full-length Gag polypeptide. Typically, the Gag peptides are of a suitable size that can be processed and/or presented by antigen-presenting cells or their precursors. A number of factors can influence the choice of peptide size. For example, the size of a peptide can be chosen such that it includes, or corresponds to the size of, CD4⁺ T cell epitopes, CD8⁺ T cell epitopes and/or B cell epitopes, and their processing requirements. Practitioners in the art will recognise that class I-restricted CD8⁺ T cell epitopes are typically between 8 and 10 amino acid residues in length and if placed next to unnatural flanking residues, such epitopes can generally require 2 to 3 natural flanking amino acid residues to ensure that they are efficiently processed and presented. Class II-restricted CD4⁺ T cell epitopes usually range between 12 and 25 amino acid residues in length and may not require natural flanking residues for efficient proteolytic processing although it is believed that natural flanking residues may play a role. Another important feature of class II-restricted epitopes is that they generally contain a core of 9-10 amino acid residues in the middle which bind specifically to class II MHC molecules with flanking sequences either side of this core stabilising binding by associating with conserved structures on either side of class II MHC antigens in a sequence independent manner. Thus the functional region of class II-restricted epitopes is typically less than about 15 amino acid residues long. The size of linear B cell epitopes and the factors effecting their processing, like class II-restricted epitopes, are quite variable although such epitopes are frequently smaller in size than 15 amino acid residues. From the foregoing, it is advantageous, but not essential, that the size of the peptide is at least 6, 7, 8, 9, 10, 11, 12, 13, 14, 15, 20, 25, 30 amino acid residues. Suitably, the size of the peptide is no more than about 500, 200, 100, 80, 60, 50, 40 amino acid residues. In some embodiments, the size of the peptide is large enough to minimise loss of T cell and/or B cell epitopes. In other embodiments, the size of the peptide is sufficient for presentation by an antigen-presenting cell of a T cell and/or a B cell epitope contained within the peptide. In an illustrative example of this type, the size of the peptide is about 15 amino acid residues.

Numerous Gap polypeptides sequences and their corresponding coding sequences are known in the art, which can be used for preparing purified, synthetic or recombinant Gag polypeptides and peptides or their coding sequence. Illustrative Gag polypeptide sequences can be obtained from any of the publicly available databases, including GenBank, EMBL and SWISSPROT. For example, representative HIV-1 Gag polypeptide sequences are available from GenBank under the following accession Nos. AAB04036, AAB03744, AAB59875, AAA44853, AAB04036, AAB50258, AAA44987, and AAB59747. Non-limiting HIV-2 Gag polypeptide sequences are available from GenBank under the following accession Nos. AAB00736, AAA76840, AAA43932, AAB00745, AAB00763, AAB01351 and AAA43941. Additionally, illustrative SIV Gag polypeptide sequences are available from GenBank under the following accession Nos. AAA91905, AAA91913, AAA47588, AAA91922, AAA74706, AAA47632, AAB59905, AAA91930 and AAB59769. Representative FIV Gag polypeptide sequences are available from GenBank under the following accession Nos. AAB59936, AAA43075 and AAB09309. A non-limiting example of a BIV Gag polypeptide sequences is available from GenBank under accession No. AAA91270. Illustrative EIAV Gag polypeptide sequences are available from GenBank under the following accession Nos.: AAB59861 and AAA43003. Non-limiting Visna Gag polypeptide sequences are available from GenBank under the following accession Nos. AAA17520, AAA48353, AAA48358, AAA17523 and AAA17528. A representative CAEV Gag polypeptide sequences is available from GenBank under accession No. AAA91825. Illustrative Ovine lentivirus Gag polypeptide sequences are available from GenBank under the following accession Nos. AAA66811 and AAA46779. It shall be understood, however, that the present invention is not limited to any specific Gag amino acid or nucleic acid sequences and extends broadly to any native or recombinant Gag polypeptides or their coding sequences.

In specific embodiments, a plurality of peptides is used to produce the Gag-specific antigen-presenting cells or their precursors, wherein individual peptides comprise different portions of an amino acid sequence corresponding to a Gag polypeptide and optionally display partial sequence identity or similarity to at least one other peptide of the plurality of peptides. The partial sequence identity or similarity is typically contained at one or both ends of an individual peptide. In one embodiment, there are at least 4, 5, 6, 7, 8, 9, 10, 11, 12, 13, 14, 15, 20, 25, 30, 40, 50 contiguous amino acid residues at one or both ends of an individual peptide, whose sequence is identical or similar to an amino acid sequence contained within at least one other of the peptides. In alternate embodiments, there are less than 500, 100, 50, 40, 30 contiguous amino acid residues at one or both ends of an individual peptide, whose sequence is identical or similar to an amino acid sequence contained within at least one other of the peptides. Such ‘sequence overlap’ is advantageous to prevent or otherwise reduce the loss of any potential epitopes contained within a Gag polypeptide. In specific examples disclosed herein, the sequence overlap is 11 amino acid residues.

In certain embodiments, the size of individual peptides is about 14 or 15 amino acid residues and the sequence overlap at one or both ends of an individual peptide is about 11 amino acid residues. However, it will be understood that other suitable peptide sizes and sequence overlap sizes are contemplated by the present invention, which can be readily ascertained by persons of skill in the art.

Typically, when peptides have partial sequence similarity, their sequences will usually differ by one or more conserved and/or non-conserved amino acid substitutions. Exemplary conservative substitutions are listed in Table 1. Conserved or non-conserved substitutions may correspond to polymorphisms within Gag. In this regard, it is well known that polymorphic Gag polypeptides are expressed by different viral strains or clades. Thus, where there a polymorphic regions in Gag, it is generally desirable to use additional sets of peptides covering the variation in amino acid residue at the polymorphic site.

It is advantageous but not necessary to utilize the entire sequence of a Gag polypeptide for producing a plurality of overlapping peptides. Typically, at least 330, 31, 32, 33, 34, 35, 36, 37, 38, 39, 40, 41, 42. 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% of the sequence corresponding to a Gag polypeptide is used to produce the overlapping peptides. However, it will be understood that the more sequence information from a Gag polypeptide that is utilised to produce the overlapping peptides, the greater the outbred population coverage will be of the overlapping peptides as an immunogen. Suitably, no sequence information from the Gag polypeptide is excluded (e.g., because of an apparent lack of immunological epitopes, since more rare or subdominant epitopes may be inadvertently missed). If required, hypervariable sequences within a Gag polypeptide can be either excluded from the construction of an overlapping set of peptides, or additional sets of peptides covering the polymorphic regions can be constructed and administered. Peptide sequences may include additional sequences that are not derived from a Gag polypeptide. These additional sequences may have various functions, including improving solubility, stability or immunogenicity or facilitating purification. Typically, such additional sequences are contained at one or both ends of a respective peptide.

Overlapping peptides may be designed based on any suitable Gag amino acid sequence, illustrative examples of which are listed above and in Tables 4 and 5. Representative overlapping peptide for modulating the immune response to simian immunodeficiency virus (SIV) and/or the chimeric SIV-HIV-1 (SHIV), both of which are known to be suitable models for the pathogenic HIV-1 virus in humans, can be based on one or more of the Gag polypeptides sequences set forth in Tables 2 and 3 infra.

Gag polypeptides and peptide may be prepared by any suitable procedure known to those of skill in the art. For example, Gag peptides can be synthesised conveniently using solution synthesis or solid phase synthesis as described, for example, in Chapter 9 of Atherton and Shephard (1989, Solid Phase Peptide Synthesis: A Practical Approach. IRL Press, Oxford) and in Roberge et al (1995, Science 269: 202). Syntheses may employ, for example, either t-butyloxycarbonyl (t-Boc) or 9-fluorenylmethyloxycarbonyl (Fmoc) chemistries (see Chapter 9.1, of Coligan et al., Current Protocols in Protein Science, John Wiley & Sons, Inc. 1995-1997; Stewart and Young, 1984, Solid Phase Peptide Synthesis, 2nd ed. Pierce Chemical Co., Rockford, Ill.; and Atherton and Shephard, supra). In specific embodiments, the individual peptides are solubilized in DMSO (e.g., 100% pure DMSO) at high concentration (1 mg peptide/10-30 μL DMSO) so that large pools of peptides do not contain excessive amounts of DMSO when pulsed onto cells. In certain advantageous embodiments, one or more peptide sets, in soluble form, are placed into a single container for convenient administration (e.g., a blood tube or vial for ready re-infusion) to a subject and such containers are also contemplated by the present invention.

Alternatively, a Gag polypeptide or peptide may be prepared by a procedure including the steps of: (a) preparing a nucleic acid construct that comprises a nucleotide sequence encoding the Gag polypeptide or peptide, wherein the nucleotide sequence is operably linked to a regulatory sequence; (b) introducing the nucleic acid construct into a suitable host cell; (c) culturing the host cell to express the nucleotide sequence; and (d) isolating the Gag polypeptide or peptide. The nucleic acid construct is typically in the form of an expression vector. For example, the expression vector can be a self-replicating extrachromosomal vector such as a plasmid, or a vector that integrates into a host genome. Typically, the regulatory sequence includes, but is not limited to, promoter sequences, leader or signal sequences, ribosomal binding sites, transcriptional start and stop sequences, translational start and termination sequences, and enhancer or activator sequences. Constitutive or inducible promoters as known in the art are contemplated by the invention. The promoters may be either naturally occurring promoters, or hybrid promoters that combine elements of more than one promoter. The regulatory sequence will generally be appropriate for the host cell used for expression. Numerous types of appropriate expression vectors and suitable regulatory polynucleotides are known in the art for a variety of host cells. In certain embodiments, the expression vector contains a selectable marker gene to allow the selection of transformed host cells. Selection genes are well known in the art and will vary with the host cell used. In other embodiments, the expression vector also includes a nucleic acid sequence that codes for a fusion partner so that Gag polypeptide or peptide is expressed as a fusion polypeptide with the fusion partner. The main advantage of fusion partners is that they assist identification and/or purification of the fusion polypeptide. Exemplary fusion partners include, but are not limited to, glutathione-S-transferase (GST), Fc portion of human IgG, maltose binding protein (MBP) and hexahistidine (HIS₆), which are particularly useful for isolation of the fusion polypeptide by affinity chromatography. For the purposes of fusion polypeptide purification by affinity chromatography, relevant matrices for affinity chromatography are glutathione-, amylose-, and nickel- or cobalt-conjugated resins respectively. Many such matrices are available in “kit” form, such as the QIAexpress™ system (Qiagen) useful with (His₆) fusion partners and the Pharmacia GST purification system. In a preferred embodiment, the recombinant polynucleotide is expressed in the commercial vector pFLAG™. Advantageously, the fusion partners also have protease cleavage sites, such as for Factor Xₐ, Thrombin and inteins (protein introns), which allow the relevant protease to partially digest the fusion polypeptide of the invention and thereby liberate the recombinant Gag polypeptide or peptide therefrom. The liberated Gag polypeptide or peptide can then be isolated from the fusion partner by subsequent chromatographic separation. Fusion partners according to the invention also include within their scope “epitope tags”, which are usually short peptide sequences for which a specific antibody is available. Well known examples of epitope tags for which specific monoclonal antibodies are readily available include c-Myc, influenza virus, haemagglutinin and FLAG tags.

The step of introducing the nucleic acid construct into the host cell may be achieved using any suitable technique including transfection, and transformation, the choice of which will be dependent on the host cell employed. Such methods are well known to those of skill in the art. The peptides of the invention may be produced by culturing a host cell transformed with the synthetic construct. The conditions appropriate for protein expression will vary with the choice of expression vector and the host cell. This is easily ascertained by one skilled in the art through routine experimentation. Suitable host cells for expression may be prokaryotic or eukaryotic. One preferred host cell for expression of a polypeptide according to the invention is a bacterium. The bacterium used may be Escherichia coli. Alternatively, the host cell may be an insect cell such as, for example, SF9 cells that may be utilised with a baculovirus expression system.

The amino acids of the Gag polypeptides or peptides can be non-naturally occurring or naturally occurring. Examples of unnatural amino acids and derivatives during peptide synthesis include but are not limited to, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, ornithine, sarcosine, 2-thienyl alanine and/or D-isomers of amino acids. A list of unnatural amino acids contemplated by the present invention is shown in Table 6.

The invention also contemplates modifying the Gag polypeptides and peptides using ordinary molecular biological techniques so as to alter their resistance to proteolytic degradation or to optimise solubility properties or to render them more suitable as an immunogenic agent.

### 3.2 Gag-Expressing Nucleic Acid Constructs for Gene Therapy

In specific embodiments, nucleic acid constructs comprising Gag coding sequences operably connected to a regulatory element, are used to make the Gag-specific antigen-presenting cells, by way of gene therapy. Gene therapy refers to therapy performed by the administration to a subject of an expressed or expressible nucleic acid. In these embodiments of the invention, the nucleic acid constructs produce their encoded Gag polypeptide or peptide(s) in an antigen-presenting cells and thereby mediate the desired therapeutic or prophylactic effect.

Any of the methods for gene therapy available in the art can be used according to the present invention. Exemplary methods are described below.

For general reviews of the methods of gene therapy, see Goldspiel et al., Clinical Pharmacy 12:488 505 (1993); Wu and Wu, Biotherapy 3:87 95 (1991); Tolstoshev, Ann. Rev. Pharmacol. Toxicol. 32:573 596 (1993); Mulligan, Science 260:926 932 (1993); and Morgan and Anderson, Ann. Rev. Biochem. 62:191 217(1993); TIBTECH11(5):155 215 (May 1993)). Methods commonly known in the art of recombinant DNA technology which can be used are described in Ausubel et al., eds., Current Protocols in Molecular Biology, John Wiley & Sons, NY (1993); and Kriegler, Gene Transfer and Expression, A Laboratory Manual, Stockton Press, NY (1990).

Delivery of the nucleic acid constructs into antigen-presenting cells or precursors may be achieved either by directly exposing a patient to the nucleic acid construct or by first transforming antigen-presenting cells or their precursors with the nucleic acid construct in vitro, and then transplanting the transformed antigen-presenting cells or precursors into the patient. These two approaches are known, respectively, as in vivo or ex vivo gene therapy.

As described, for example, in U.S. Pat. No. 5,976,567 (Inex), the expression of natural or synthetic nucleic acids is typically achieved by operably linking a nucleic acid of interest to a promoter (which may be either constitutive or inducible), usually incorporating the construct into an expression vector, and introducing the vector into a suitable host cell. Typical vectors contain transcription and translation terminators, transcription and translation initiation sequences, and promoters useful for regulation of the expression of the particular nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors may be suitable for replication and integration in prokaryotes, eukaryotes, or preferably both. See, Giliman and Smith (1979), Gene, 8: 81-97; Roberts et al. (1987), Nature, 328: 731-734; Berger and Kimmel, Guide to Molecular Cloning Techniques, Methods in Enzymology, volume 152, Academic Press, Inc., San Diego, Calif. (Berger); Sambrook et al. (1989), Molecular Cloning—A Laboratory Manual (2nd ed.) Vol. 1-3, Cold Spring Harbor Laboratory, Cold Spring Harbor Press, N.Y., (Sambrook); and F. M. Ausubel et al., Current Protocols in Molecular Biology, eds., Current Protocols, a joint venture between Greene Publishing Associates, Inc. and John Wiley & Sons, Inc., (1994 Supplement) (Ausubel).

Expression vectors containing regulatory elements from eukaryotic viruses such as retroviruses are typically used for expression of nucleic acid sequences in eukaryotic cells. SV40 vectors include pSVT7 and pMT2. Vectors derived from bovine papilloma virus include pBV-1MTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

While a variety of vectors may be used, it should be noted that viral expression vectors are useful for modifying eukaryotic cells because of the high efficiency with which the viral vectors transfect target cells and integrate into the target cell genome. Illustrative expression vectors of this type can be derived from viral DNA sequences including, but not limited to, adenovirus, adeno-associated viruses, herpes-simplex viruses and retroviruses such as B, C, and D retroviruses as well as spumaviruses and modified lentiviruses. Suitable expression vectors for transfection of animal cells are described, for example, by Wu and Ataai (2000, Curr. Opin. Biotechnol. 11(2), 205-208), Vigna and Naldini (2000, J. Gene Med. 2(5), 308-316), Kay et al. (2001, Nat. Med. 7(1), 33-40), Athanasopoulos, et al. (2000, Int. J. Mol. Med. 6(4),363-375) and Walther and Stein (2000, Drugs 60(2), 249-271).

The Gag-encoding portion of the expression vector may comprise a naturally-occurring sequence or a variant thereof, which has been engineered using recombinant techniques. In one example of a variant, the codon composition of an antigen-encoding polynucleotide is modified to permit enhanced expression of the antigen in a target cell or tissue of choice using methods as set forth in detail in International Publications WO 99/02694 and WO 00/42215. Briefly, these methods are based on the observation that translational efficiencies of different codons vary between different cells or tissues and that these differences can be exploited, together with codon composition of a gene, to regulate expression of a protein in a particular cell or tissue type. Thus, for the construction of codon-optimised polynucleotides, at least one existing codon of a parent polynucleotide is replaced with a synonymous codon that has a higher translational efficiency in a target cell or tissue than the existing codon it replaces. Although it is desirable to replace all the existing codons of a parent nucleic acid molecule with synonymous codons which have that higher translational efficiency, this is not necessary because increased expression can be accomplished even with partial replacement. Suitably, the replacement step affects 5%, 10%, 15%, 20%, 25%, 30%, more preferably 35%, 40%, 50%, 60%, 70% or more of the existing codons of a parent polynucleotide.

The expression vector is compatible with the antigen-presenting cell or precursor in which it is introduced such that the antigen-encoding polynucleotide is expressible in that cell or precursor. The expression vector is introduced into the antigen-presenting cell or precursor by any suitable means which will be dependent on the particular choice of expression vector and antigen-presenting cell employed. Such means of introduction are well-known to those skilled in the art. For example, introduction can be effected by use of contacting (e.g., in the case of viral vectors), electroporation, transformation, transduction, conjugation or triparental mating, transfection, infection membrane fusion with cationic lipids, high-velocity bombardment with DNA-coated microprojectiles, incubation with calcium phosphate-DNA precipitate, direct microinjection into single cells, and the like. Other methods also are available and are known to those skilled in the art. Alternatively, the vectors are introduced by means of cationic lipids, e.g., liposomes. Such liposomes are commercially available (e.g., Lipofectin®, Lipofectamine™, and the like, supplied by Life Technologies, Gibco BRL, Gaithersburg, Md.).

In other embodiments, the nucleic acid construct is introduced into antigen-presenting cells or their precursors by administering it in linkage to a ligand subject to receptor-mediated endocytosis (see, e.g., Wu and Wu, J. Biol. Chem. 262:4429 4432 (1987)) (which can be used to target cell types specifically expressing the receptors), etc. In still other embodiments, nucleic acid-ligand complexes can be formed in which the ligand comprises a fusogenic viral peptide to disrupt endosomes, allowing the nucleic acid construct to avoid lysosomal degradation. In yet other embodiments, the nucleic acid construct can be targeted in vivo for cell specific uptake and expression, by targeting a specific receptor (see, e.g., PCT Publications WO 92/06180 dated Apr. 16, 1992 (Wu et al.); WO 92/22635 dated Dec. 23, 1992 (Wilson et al.); WO92/20316 dated Nov. 26, 1992 (Findeis et al.); WO093/14188 dated Jul. 22, 1993 (Clarke et al.); and WO 93/20221 dated Oct. 14, 1993 (Young)). Alternatively, the nucleic acid can be introduced intracellularly and incorporated within host cell DNA for expression, by homologous recombination (Koller and Smithies, Proc. Natl. Acad. Sci. USA 86:8932 8935 (1989); Zijlstra et al., Nature 342:435 438 (1989)).

Another approach to gene therapy involves transferring the nucleic acid construct to cells in tissue culture, which usually includes transfer of a selectable marker to the cells. The cells are then placed under selection to isolate those cells that have taken up and are expressing the transferred gene. Those cells are then delivered to a patient. In this embodiment, the nucleic acid construct is introduced into antigen-presenting cells or precursors prior to administration in vivo of the resulting recombinant cells. Such introduction can be carried out by any method known in the art, including, but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, Meth. Enzymol. 217:599 618 (1993); Cohen et al., Meth. Enzymol. 217:618 644 (1993); Cline, Pharmac. Ther. 29:69 92 (1985)) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny. The resulting recombinant cells can be delivered to a patient by various methods known in the art. Recombinant antigen-presenting cells are typically administered intravenously.

### 3.3 Particle Embodiments

In some embodiments, the Gag polypeptide(s) or peptide(s) as broadly described in Section 3.1 or the Gag-expressing nucleic acid constructs as broadly described in Section 3.2 (also referred to herein as “immune stimulators” or “Gag immune stimulators”) are provided in particulate form (also referred to herein as “Gag particles”). These embodiments are particularly advantageous for delivering the immune stimulators to antigen-presenting cells or their precursors, either ex vivo or in vivo, since particles are preferentially taken up (e.g., by endocytosis or phagocytosis) by such cells. A variety of particles may be used in the invention, including but not limited to, liposomes, micelles, lipidic particles, ceramic/inorganic particles and polymeric particles, and are typically selected from nanoparticles and microparticles. The particles are suitably sized for phagocytosis or endocytosis by antigen-presenting cells or their precursors.

Antigen-presenting cells include both professional and facultative types of antigen-presenting cells. Professional antigen-presenting cells include, but are not limited to, macrophages, monocytes, B lymphocytes, cells of myeloid lineage, including monocytic-granulocytic-DC precursors, marginal zone Kupffer cells, microglia, T cells, Langerhans cells and dendritic cells including interdigitating dendritic cells and follicular dendritic cells. Examples of facultative antigen-presenting cells include but are not limited to activated T cells, astrocytes, follicular cells, endothelium and fibroblasts. In some embodiments, the antigen-presenting cell is selected from monocytes, macrophages, B-lymphocytes, cells of myeloid lineage, dendritic cells or Langerhans cells. In specific embodiments, the antigen-presenting cell expresses CD11c and includes a dendritic cell. In illustrative examples, the particles have a dimension of less than about 100 μm, more suitably in the range of less than or equal to about 500 nm, although the particles may be as large as about 10 μm, and as small as a few nm. Liposomes consist basically of a phospholipid bilayer forming a shell around an aqueous core. Advantages include the lipophilicity of the outer layers which “mimic” the outer membrane layers of cells and that they are taken up relatively easily by a variety of cells. Polymeric vehicles typically consist of micro/nanospheres and micro/nanocapsules formed of biocompatible polymers, which are either biodegradable (for example, polylactic acid) or non-biodegradable (for example, ethylenevinyl acetate). Some of the advantages of the polymeric devices are ease of manufacture and high loading capacity, range of size from nanometer to micron diameter, as well as controlled release and degradation profile.

In some embodiments, the particles comprise an antigen-binding molecule on their surface, which is immuno-interactive with a marker that is expressed at higher levels on antigen-presenting cells (e.g., dendritic cells) than on non-antigen-presenting cells. Illustrative markers of this type include MGL, DCL-1, DEC-205, macrophage mannose R, DC-SIGN or other DC or myeloid specific (lectin) receptors, as for example disclosed by Hawiger et al. (2001, J Exp Med 194, 769), Kato et al. 2003, J Biol Chem 278, 34035), Benito et al. (2004, J Am Chem Soc 126, 10355), Schjetne, et al. (2002, Int Immunol 14, 1423) and van Vliet et al., 2006, Nat Immunol September 24; [Epub ahead of print])(van Vliet et al., Immunobiology 2006, 211:577-585).

The particles can be prepared from a combination of the immune stimulator(s), and a surfactant, excipient or polymeric material. In some embodiments, the particles are biodegradable and biocompatible, and optionally are capable of biodegrading at a controlled rate for delivery of a therapeutic or diagnostic agent. The particles can be made of a variety of materials. Both inorganic and organic materials can be used. Polymeric and non-polymeric materials, such as fatty acids, may be used. Other suitable materials include, but are not limited to, gelatin, polyethylene glycol, trehalulose, dextran and chitosan. Particles with degradation and release times ranging from seconds to months can be designed and fabricated, based on factors such as the particle material.

### 3.3.1 Polymeric Particles

Polymeric particles may be formed from any biocompatible and desirably biodegradable polymer, copolymer, or blend. The polymers may be tailored to optimize different characteristics of the particle including: i) interactions between the immune stimulators to be delivered and the polymer to provide stabilization of the immune stimulators and retention of activity upon delivery; ii) rate of polymer degradation and, thereby, rate of agent release profiles; iii) surface characteristics and targeting capabilities via chemical modification; and iv) particle porosity.

Surface eroding polymers such as polyanhydrides may be used to form the particles. For example, polyanhydrides such as poly[(p-carboxyphenoxy)-hexane anhydride] (PCPH) may be used. Biodegradable polyanhydrides are described in U.S. Pat. No. 4,857,311.

In other embodiments, bulk eroding polymers such as those based on polyesters including poly(hydroxy acids) or poly(esters) can be used. For example, polyglycolic acid (PGA), polylactic acid (PLA), or copolymers thereof may be used to form the particles. The polyester may also have a charged or functionalizable group, such as an amino acid. In illustrative examples, particles with controlled release properties can be formed of poly(D,L-lactic acid) and/or poly(D,L-lactic-co-glycolic acid) (“PLGA”) which incorporate a surfactant such as DPPC.

Other polymers include poly(alkylcyanoacrylates), polyamides, polycarbonates, polyalkylenes such as polyethylene, polypropylene, poly(ethylene glycol), poly(ethylene oxide), poly(ethylene terephthalate), poly vinyl compounds such as polyvinyl alcohols, polyvinyl ethers, and polyvinyl esters, polymers of acrylic and methacrylic acids, celluloses and other polysaccharides, and peptides or proteins, or copolymers or blends thereof. Polymers may be selected with or modified to have the appropriate stability and degradation rates in vivo for different controlled drug delivery applications.

In some embodiments, particles are formed from functionalized polyester graft copolymers, as described in Hrkach et al. (1995, Macromolecules, 28:4736-4739; and “Poly(L-Lactic acid-co-amino acid) Graft Copolymers: A Class of Functional Biodegradable Biomaterials” in Hydrogels and Biodegradable Polymers for Bioapplications, ACS Symposium Series No. 627, Raphael M. Ottenbrite et al., Eds., American Chemical Society, Chapter 8, pp. 93-101, 1996.)

Materials other than biodegradable polymers may be used to form the particles. Suitable materials include various non-biodegradable polymers and various excipients. The particles also may be formed of the immune stimulator(s) and surfactant alone.

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art, provided that the conditions are optimized for forming particles with the desired diameter.

Methods developed for making microspheres for delivery of encapsulated agents are described in the literature, for example, as described in Doubrow, M., Ed., “Microcapsules and Nanoparticles in Medicine and Pharmacy,” CRC Press, Boca Raton, 1992. Methods also are described in Mathiowitz and Langer (1987, J. Controlled Release 5, 13-22); Mathiowitz et al. (1987, Reactive Polymers 6, 275-283); and Mathiowitz et al. (1988, J. Appl. Polymer Sci. 35, 755-774) as well as in U.S. Pat. No. 5,213,812, U.S. Pat. No. 5,417,986, U.S. Pat. No. 5,360,610, and U.S. Pat. No. 5,384,133. The selection of the method depends on the polymer selection, the size, external morphology, and crystallinity that is desired, as described, for example, by Mathiowitz et al. (1990, Scanning Microscopy 4: 329-340; 1992, J. Appl. Polymer Sci. 45, 125-134); and Benita et al. (1984, J. Pharm. Sci. 73, 1721-1724).

In solvent evaporation, described for example, in Mathiowitz et al., (1990), Benita; and U.S. Pat. No. 4,272,398 to Jaffe, the polymer is dissolved in a volatile organic solvent, such as methylene chloride. Several different polymer concentrations can be used, for example, between 0.05 and 2.0 g/mL. The immune stimulator(s), either in soluble form or dispersed as fine particles, is (are) added to the polymer solution, and the mixture is suspended in an aqueous phase that contains a surface-active agent such as poly(vinyl alcohol). The aqueous phase may be, for example, a concentration of 1% poly(vinyl alcohol) w/v in distilled water. The resulting emulsion is stirred until most of the organic solvent evaporates, leaving solid microspheres, which may be washed with water and dried overnight in a lyophilizer. Microspheres with different sizes (between 1 and 1000 μm) and morphologies can be obtained by this method.

Solvent removal was primarily designed for use with less stable polymers, such as the polyanhydrides. In this method, the agent is dispersed or dissolved in a solution of a selected polymer in a volatile organic solvent like methylene chloride. The mixture is then suspended in oil, such as silicon oil, by stirring, to form an emulsion. Within 24 hours, the solvent diffuses into the oil phase and the emulsion droplets harden into solid polymer microspheres. Unlike the hot-melt microencapsulation method described for example in Mathiowitz et al. (1987, Reactive Polymers, 6:275), this method can be used to make microspheres from polymers with high melting points and a wide range of molecular weights. Microspheres having a diameter for example between one and 300 μm can be obtained with this procedure.

With some polymeric systems, polymeric particles prepared using a single or double emulsion technique, vary in size depending on the size of the droplets. If droplets in water-in-oil emulsions are not of a suitably small size to form particles with the desired size range, smaller droplets can be prepared, for example, by sonication or homogenation of the emulsion, or by the addition of surfactants.

If the particles prepared by any of the above methods have a size range outside of the desired range, particles can be sized, for example, using a sieve, and further separated according to density using techniques known to those of skill in the art.

The polymeric particles can be prepared by spray drying. Methods of spray drying, such as that disclosed in PCT WO 96/09814 by Sutton and Johnson, disclose the preparation of smooth, spherical microparticles of a water-soluble material with at least 90% of the particles possessing a mean size between 1 and 10 μm.

### 3.3.2 Ceramic Particles

Ceramic particles may also be used to deliver the immune stimulators of the invention. These particles are typically prepared using processes similar to the well known sol-gel process and usually require simple and room temperature conditions as described for example in Brinker et al. (“Sol-Gel Science: The Physics and Chemistry of Sol-Gel Processing;” Academic Press: San Diego, 1990, p-60), and Avnir et al. (1994, Chem. Mater. 6, 1605). Ceramic particles can be prepared with desired size, shape and porosity, and are extremely stable. These particles also effectively protect doped molecules (polypeptides, drugs etc.) against denaturation induced by extreme pH and temperature (Jain et al., 1998, J. Am. Chem. Soc. 120, 11092-11095). In addition, their surfaces can be easily functionalized with different groups (Lal et al., 2000, Chem. Mater. 12, 2632-2639; Badley et al., 1990, Langmuir, 6, 792-801), and therefore they can be attached to a variety of monoclonal antibodies and other ligands in order to target them to desired sites in vivo.

Various ceramic particles have been described for delivery in vivo of active agent-containing payloads. For example, British Patent 1 590 574 discloses incorporation of biologically active components in a sol-gel matrix. International Publication WO 97/45367 discloses controllably dissolvable silica xerogels prepared via a sol-gel process, into which a biologically active agent is incorporated by impregnation into pre-sintered particles (1 to 500 μm) or disks. International Publication WO 0050349 discloses controllably biodegradable silica fibres prepared via a sol-gel process, into which a biologically active agent is incorporated during synthesis of the fibre. U.S. Pat. Appl. Pub. 20040180096 describes ceramic nanoparticles in which a bioactive substance is entrapped. The ceramic nanoparticles are made by formation of a micellar composition of the dye. The ceramic material is added to the micellar composition and the ceramic nanoparticles are precipitated by alkaline hydrolysis. U.S. Pat. Appl. Pub. 20050123611 discloses controlled release ceramic particles comprising an active material substantially homogeneously dispersed throughout the particles. These particles are prepared by mixing a surfactant with an apolar solvent to prepare a reverse micelle solution; (b) dissolving a gel precursor, a catalyst, a condensing agent and a soluble active material in a polar solvent to prepare a precursor solution; (c) combining the reverse micelle solution and the precursor solution to provide an emulsion and (d) condensing the precursor in the emulsion. U.S. Pat. Appl. Pub. 20060210634 discloses adsorbing bioactive substances onto ceramic particles comprising a metal oxide (e.g., titanium oxide, zirconium oxide, scandium oxide, cerium oxide and yttrium oxide) by evaporation. Kortesuo et al. (2000, Int J Pharm. May 10; 200(2):223-229) disclose a spray drying method to produce spherical silica gel particles with a narrow particle size range for controlled delivery of drugs such as toremifene citrate and dexmedetomidine HCl. Wang et al. (2006, Int J Pharm. 308(1-2):160-167) describe the combination of adsorption by porous CaCO₃ microparticles and encapsulation by polyelectrolyte multilayer films for delivery of bioactive substances.

### 3.3.3 Liposomes

Liposomes can be produced by standard methods such as those reported by Kim et al. (1983, Biochim. Biophys. Acta 728, 339-348); Liu et al. (1992, Biochim. Biophys. Acta 1104, 95-101); Lee et al. (1992, Biochim. Biophys. Acta. 1103, 185-197), Brey et al. (U.S. Pat. Appl. Pub. 20020041861), Hass et al. (U.S. Pat. Appl. Pub. 20050232984), Kisak et al. (U.S. Pat. Appl. Pub. 20050260260) and Smyth-Templeton et al. (U.S. Pat. Appl. Pub. 20060204566). Additionally, reference may be made to Copeland et al. (2005, Immunol. Cell Biol. 83: 95-105) who review lipid based particulate formulations for the delivery of antigen, and to Bramwell et al. (2005, Crit Rev Ther Drug Carrier Syst. 22(2):151-214; 2006, J Pharm Pharmacol. 58(6):717-728) who review particulate delivery systems for vaccines, including methods for the preparation of protein-loaded liposomes. Many liposome formulations using a variety of different lipid components have been used in various in vitro cell culture and animal experiments. Parameters have been identified that determine liposomal properties and are reported in the literature, for example, by Lee et al. (1992, Biochim. Biophys. Acta. 1103, 185-197); Liu et al. (1992, Biochim. Biophys. Acta, 1104, 95-101); and Wang et al. (1989, Biochem. 28, 9508-951).

Briefly, the lipids of choice (and any organic-soluble bioactive), dissolved in an organic solvent, are mixed and dried onto the bottom of a glass tube under vacuum. The lipid film is rehydrated using an aqueous buffered solution containing any water-soluble bioactives to be encapsulated by gentle swirling. The hydrated lipid vesicles can then be further processed by extrusion, submitted to a series of freeze-thawing cycles or dehydrated and then rehydrated to promote encapsulation of bioactives. Liposomes can then be washed by centrifugation or loaded onto a size-exclusion column to remove unentrapped bioactive from the liposome formulation and stored at 4° C. The basic method for liposome preparation is described in more detail in Thierry et al. (1992, Nuc. Acids Res. 20:5691-5698).

A particle carrying a payload of immune stimulator(s) can be made using the procedure as described in: Pautot et al. (2003, Proc. Natl. Acad. Sci. USA, 100(19):10718-21). Using the Pautot et al. technique, streptavidin-coated lipids (DPPC, DSPC, and similar lipids) can be used to manufacture liposomes. The drug encapsulation technique described by Needham et al. (2001, Advanced Drug Delivery Reviews, 53(3): 285-305) can be used to load these vesicles with one or more active agents.

The liposomes can be prepared by exposing chloroformic solution of various lipid mixtures to high vacuum and subsequently hydrating the resulting lipid films (DSPC/CHOL) with pH 4 buffers, and extruding them through polycarbonated filters, after a freezing and thawing procedure. It is possible to use DPPC supplemented with DSPC or cholesterol to increase encapsulation efficiency or increase stability, etc. A transmembrane pH gradient is created by adjusting the pH of the extravesicular medium to 7.5 by addition of an alkalinization agent. A Gag immune stimulator can be subsequently entrapped by addition of a solution of the immune stimulator in small aliquots to the vesicle solution, at an elevated temperature, to allow accumulation of the immune stimulator inside the liposomes.

Other lipid-based particles suitable for the delivery of the immune stimulators of the present invention such as niosomes are described by Copeland et al. (2005, Immunol. Cell Biol. 83: 95-105).

### 3.3.4 Ballistic Particles

The immune stimulators of the present invention may be attached to (e.g., by coating or conjugation) or otherwise associated with particles suitable for use in needleless or “ballistic” (biolistic) delivery. Illustrative particles for ballistic delivery are described, for example, in: International Publications WO 02/101412; WO 02/100380; WO 02/43774; WO 02/19989; WO 01/93829; WO 01/83528; WO 00/63385; WO 00/26385; WO 00/19982; WO 99/01168; WO 98/10750; and WO 97/48485. It shall be understood, however, that such particles are not limited to their use with a ballistic delivery device and can otherwise be administered by any alternative technique (e.g., injection or microneedle delivery) through which particles are deliverable to immune cells.

The immune stimulators can be coated or chemically coupled to carrier particles (e.g., core carriers) using a variety of techniques known in the art. Carrier particles are selected from materials which have a suitable density in the range of particle sizes typically used for intracellular delivery. The optimum carrier particle size will, of course, depend on the diameter of the target cells. Illustrative particles have a size ranging from about 0.01 to about 250 μm, from about 10 to about 150 μm, and from about 20 to about 60 μm; and a particle density ranging from about 0.1 to about 25 g/cm³, and a bulk density of about 0.5 to about 3.0 g/cm³, or greater. Non-limiting particles of this type include metal particles such as, tungsten, gold, platinum and iridium carrier particles. Tungsten particles are readily available in average sizes of 0.5 to 2.0 μm in diameter. Gold particles or microcrystalline gold (e.g., gold powder A1570, available from Engelhard Corp., East Newark, N.J.) may also be used. Gold particles provide uniformity in size (available from Alpha Chemicals in particle sizes of 1-3 μm, or available from Degussa, South Plainfield, N.J. in a range of particle sizes including 0.95 μm) and low toxicity. Microcrystalline gold provides a diverse particle size distribution, typically in the range of 0.1-5 μm. The irregular surface area of microcrystalline gold provides for highly efficient coating with the active agents of the present invention.

Many methods are known and have been described for adsorbing, coupling or otherwise attaching bioactive molecules (e.g., hydrophilic molecules such as proteins and nucleic acids) onto particles such as gold or tungsten particles. In illustrative examples, such methods combine a predetermined amount of gold or tungsten with the bioactive molecules, CaCl₂ and spermidine. In other examples, ethanol is used to precipitate the bioactive molecules onto gold or tungsten particles (see, for example, Jumar et al., 2004, Phys Med. Biol. 49:3603-3612). The resulting solution is suitably vortexed continually during the coating procedure to ensure uniformity of the reaction mixture. After attachment of the bioactive molecules, the particles can be transferred for example to suitable membranes and allowed to dry prior to use, coated onto surfaces of a sample module or cassette, or loaded into a delivery cassette for use in particular particle-mediated delivery instruments.

The formulated compositions may suitably be prepared as particles using standard techniques, such as by simple evaporation (air drying), vacuum drying, spray drying, freeze drying (lyophilization), spray-freeze drying, spray coating, precipitation, supercritical fluid particle formation, and the like. If desired, the resultant particles can be dandified using the techniques described in International Publication WO 97/48485.

### 3.3.5 Surfactants

Surfactants which can be incorporated into particles include phosphoglycerides. Exemplary phosphoglycerides include phosphatidylcholines, such as the naturally occurring surfactant, L-α-phosphatidylcholine dipalmitoyl (“DPPC”). The surfactants advantageously improve surface properties by, for example, reducing particle-particle interactions, and can render the surface of the particles less adhesive. The use of surfactants endogenous to the lung may avoid the need for the use of non-physiologic surfactants.

Providing a surfactant on the surfaces of the particles can reduce the tendency of the particles to agglomerate due to interactions such as electrostatic interactions, Van der Waals forces, and capillary action. The presence of the surfactant on the particle surface can provide increased surface rugosity (roughness), thereby improving aerosolization by reducing the surface area available for intimate particle-particle interaction.

Surfactants known in the art can be used including any naturally occurring surfactant. Other exemplary surfactants include diphosphatidyl glycerol (DPPG); hexadecanol; fatty alcohols such as polyethylene glycol (PEG); polyoxyethylene-9-lauryl ether; a surface active fatty acid, such as palmitic acid or oleic acid; sorbitan trioleate (Span 85); glycocholate; surfactin; a poloxamer; a sorbitan fatty acid ester such as sorbitan trioleate; tyloxapol and a phospholipid.

### 3.4 Antigen-Presenting Cell Embodiments

In some embodiments, the immune stimulator that is used to increase the number of Gag-specific antigen-presenting cells in the subject is an antigen-presenting cell or its precursor, which is obtained from the subject to be treated (i.e., an autologous antigen-presenting cell or precursor) or from a donor that is MHC matched or mismatched with the subject (i.e., an allogeneic antigen-presenting cell). Desirably, the donor is histocompatible with the subject. In these embodiments, a Gag-specific antigen-presenting cell or precursor is produced by contacting the antigen-presenting cell or precursor with (i) a Gag polypeptide or a Gag peptide as described for example in Section 3.1, which is suitably in soluble form or in particulate form as described for example in Section 3.3, or with (ii) a Gag-expressing nucleic acid construct as described for example in Section 3.2, which is suitably in soluble form or in particulate form as described for example in Section 3.3, in an amount and for a time sufficient for a Gag peptide to be presented by the antigen-presenting cell or precursor on its surface.

### 3.4.1 Sources of Antigen Presenting Cells and Their Precursors

Antigen-presenting cells or their precursors can be isolated by methods known to those of skill in the art. The source of such cells will differ depending upon the antigen-presenting cell required for modulating a specified immune response. In this context, the antigen-presenting cell can be selected from dendritic cells, macrophages, monocytes and other cells of myeloid lineage.

Typically, precursors of antigen-presenting cells can be isolated from any tissue, but are most easily isolated from blood, cord blood or bone marrow (Sorg et al., 2001, Exp Hematol 29, 1289-1294; Zheng et al., 2000, J Hematother Stem Cell Res 9, 453-464). It is also possible to obtain suitable precursors from diseased tissues such as rheumatoid synovial tissue or fluid following biopsy or joint tap (Thomas et al., 1994a, J Immunol 153, 4016-4028; Thomas et al., 1994b, Arthritis Rheum 37(4)). Other examples include, but are not limited to liver, spleen, heart, kidney, gut and tonsil (Lu et al., 1994, J Exp Med 179, 1823-1834; McIlroy et al., 2001, Blood 97, 3470-3477; Vremec et al., 2000, J Immunol 159, 565-573; Hart and Fabre, 1981, J Exp Med 154(2), 347-361; Hart and McKenzie, 1988, J Exp Med 168(1), 157-170; Pavli et al., 1990, Immunology 70(1), 40-47).

Leukocytes isolated directly from tissue provide a major source of antigen-presenting cell precursors. Typically, these precursors can only differentiate into antigen-presenting cells by culturing in the presence or absence of various growth factors. According to the practice of the present invention, the antigen-presenting cells may be so differentiated from crude mixtures or from partially or substantially purified preparations of precursors. Leukocytes can be conveniently purified from blood or bone marrow by density gradient centrifugation using, for example, Ficoll Hypaque which eliminates neutrophils and red cells (peripheral blood mononuclear cells or PBMCs), or by ammonium chloride lysis of red cells (leukocytes or white blood cells). Many precursors of antigen-presenting cells are present in peripheral blood as non-proliferating monocytes, which can be differentiated into specific antigen-presenting cells, including macrophages and dendritic cells, by culturing in the presence of specific cytokines.

Tissue-derived precursors such as precursors of tissue dendritic cells or of Langerhans cells are typically obtained by mincing tissue (e.g., basal layer of epidermis) and digesting it with collagenase or dispase followed by density gradient separation, or selection of precursors based on their expression of cell surface markers. For example, Langerhans cell precursors express CD1 molecules as well as HLA-DR and can be purified on this basis.

In some embodiments, the antigen-presenting cell precursor is a precursor of macrophages. Generally these precursors can be obtained from monocytes of any source and can be differentiated into macrophages by prolonged incubation in the presence of medium and macrophage colony stimulating factor (M-CSF) (Erickson-Miller et al., 1990, Int J Cell Cloning 8, 346-356; Metcalf and Burgess, 1982, J Cell Physiol, 111, 275-283).

In other embodiments, the antigen presenting cell precursor is a precursor of Langerhans cells. Usually, Langerhans cells can be generated from human monocytes or CD34⁺ bone marrow precursors in the presence of granulocyte/macrophage colony-stimulating factor (GM-CSF), IL-4/TNFα and TGFβ (Geissmann et al., 1998, J Exp Med, 187, 961-966; Strobl et al., 1997a, Blood 90, 1425-1434; Strobl et al., 1997b, dv Exp Med Biol 417, 161-165; Strobl et al., 1996, J Immunol 157, 1499-1507).

In still other embodiments, the antigen-presenting cell precursor is a precursor of dendritic cells. Several potential dendritic cell precursors can be obtained from peripheral blood, cord blood or bone marrow. These include monocytes, CD34⁺ stem cells, granulocytes, CD33⁺CD11c⁺ DC precursors, and committed myeloid progenitors—described below.

Monocytes:

Monocytes can be purified by adherence to plastic for 1-2 h in the presence of tissue culture medium (e.g., RPMI) and serum (e.g., human or foetal calf serum), or in serum-free medium (Anton et al., 1998, Scand J Immunol 47, 116-121; Araki et al., 2001, Br J Haematol 114, 681-689; Mackensen et al., 2000, Int J Cancer 86, 385-392; Nestle et al., 1998, Nat Med 4, 328-332; Romani et al., 1996, J Immunol Meth 196, 137-151; Thurner et al., 1999, J Immunol Methods 223, 1-15). Monocytes can also be elutriated from peripheral blood (Garderet et al., 2001, J Hematother Stem Cell Res 10, 553-567). Monocytes can also be purified by immunoaffinity techniques, including immunomagnetic selection, flow cytometric sorting or panning (Araki et al., 2001, supra; Battye and Shortman, 1991, Curr. Opin. Immunol. 3, 238-241), with anti-CD14 antibodies to obtain CD14hi cells. The numbers (and therefore yield) of circulating monocytes can be enhanced by the in vivo use of various cytokines including GM-CSF (Groopman et al., 1987, N Engl J Med 317, 593-598; Hill et al., 1995, J Leukoc Biol 58, 634-642). Monocytes can be differentiated into dendritic cells by prolonged incubation in the presence of GM-CSF and IL-4 (Romani et al., 1994, J Exp Med 180, 83-93; Romani et al., 1996, supra). A combination of GM-CSF and IL-4 at a concentration of each at between about 200 to about 2000 U/mL, more preferably between about 500 to about 1000 U/mL and even more preferably between about 800 U/mL (GM-CSF) and 1000 U/mL (IL-4) produces significant quantities of immature dendritic cells, i.e., antigen-capturing phagocytic dendritic cells. Other cytokines which promote differentiation of monocytes into antigen-capturing phagocytic dendritic cells include, for example, IL-13.

CD34+ Stem Cells:

Dendritic cells can also be generated from CD34⁺ bone marrow derived precursors in the presence of GM-CSF, TNFα±stem cell factor (SCF, c-kitL), or GM-CSF, IL-4±flt3L (Bai et al., 2002, Int J Oncol 20, 247-53; Chen et al., 2001, Clin Immunol 98, 280-292; Loudovaris et al., 2001, J Hematother Stem Cell Res 10, 569-578). CD34⁺ cells can be derived from a bone marrow aspirate or from blood and can be enriched as for monocytes using, for example, immunomagnetic selection or immunocolumns (Davis et al., 1994, J Immunol Meth 175, 247-257). The proportion of CD34⁺ cells in blood can be enhanced by the in vivo use of various cytokines including (most commonly) G-CSF, but also flt3L and progenipoietin (Fleming et al., 2001, Exp Hematol 29, 943-951; Pulendran et al., 2000, J Immunol 165, 566-572; Robinson et al., 2000, J Hematother Stem Cell Res 9, 711-720).

Other Myeloid Progenitors:

DC can be generated from committed early myeloid progenitors in a similar fashion to CD34+ stem cells, in the presence of GM-CSF and IL-4/TNF. Such myeloid precursors infiltrate many tissues in inflammation, including rheumatoid arthritis synovial fluid (Santiago-Schwarz et al., 2001, J Immunol. 167, 1758-1768). Expansion of total body myeloid cells including circulating dendritic cell precursors and monocytes, can be achieved with certain cytokines, including flt-3 ligand, granulocyte colony-stimulating factor (G-CSF) or progenipoietin (pro-GP) (Fleming et al., 2001, supra; Pulendran et al., 2000, supra; Robinson et al., 2000, supra). Administration of such cytokines for several days to a human or other mammal would enable much larger numbers of precursors to be derived from peripheral blood or bone marrow for in vitro manipulation. Dendritic cells can also be generated from peripheral blood neutrophil precursors in the presence of GM-CSF, IL-4 and TNFα (Kelly et al., 2001, Cell Mol Biol (Noisy-le-grand) 47, 43-54; Oehler et al., 1998, J Exp Med. 187, 1019-1028). It should be noted that dendritic cells can also be generated, using similar methods, from acute myeloid leukaemia cells (Oehler et al., 2000, Ann Hematol. 79, 355-62).

Tissue DC Precursors and Other Sources of APC Precursors:

Other methods for DC generation exist from, for example, thymic precursors in the presence of IL-3+/−GM-CSF, and liver DC precursors in the presence of GM-CSF and a collagen matrix. Transformed or immortalised dendritic cell lines may be produced using oncogenes such as v-myc as for example described by (Paglia et al., 1993) or by myb (Banyer and Hapel, 1999; Gonda et al., 1993).

Circulating DC Precursors:

These have been described in human and mouse peripheral blood. One can also take advantage of particular cell surface markers for identifying suitable dendritic cell precursors. Specifically, various populations of dendritic cell precursors can be identified in blood by the expression of CD11c and the absence or low expression of CD14, CD19, CD56 and CD3 (O'Doherty et al., 1994, Immunology 82, 487-493; O'Doherty et al., 1993, J Exp Med 178, 1067-1078). These cells can also be identified by the cell surface markers CD13 and CD33 (Thomas et al., 1993b, J Immunol 151(12), 6840-6852). A second subset, which lacks CD14, CD19, CD56 and CD3, known as plasmacytoid dendritic cell precursors, does not express CD11c, but does express CD123 (IL-3R chain) and HLA-DR (Farkas et al., 2001, Am J Pathol 159, 237-243; Grouard et al., 1997, J Exp Med 185, 1101-1111; Rissoan et al., 1999, Science 283, 1183-1186). Most circulating CD11⁺ dendritic cell precursors are HLA-DR⁺, however some precursors may be HLA-DR-. The lack of MHC class II expression has been clearly demonstrated for peripheral blood dendritic cell precursors (del Hoyo et al., 2002, Nature 415, 1043-1047).

Optionally, CD33⁺CD14^{-/lo} or CD11c⁺HLA-DR⁺, lineage marker-negative dendritic cell precursors described above can be differentiated into more mature antigen-presenting cells by incubation for 18-36 h in culture medium or in monocyte conditioned medium (Thomas et al., 1993b, supra; Thomas and Lipsky, 1994, J Immunol 153, 4016-4028) (O′Doherty et al., 1993, supra). Alternatively, following incubation of peripheral blood non-T cells or unpurified PBMC, the mature peripheral blood dendritic cells are characterised by low density and so can be purified on density gradients, including metrizamide and Nycodenz (Freudenthal and Steinman, 1990, Proc Natl Acad Sci U S A 87, 7698-7702; Vremec and Shortman, 1997, J Immunol 159, 565-573), or by specific monoclonal antibodies, such as but not limited to the CMRF-44 mAb (Fearnley et al., 1999, Blood 93, 728-736; Vuckovic et al., 1998, Exp Hematol 26, 1255-1264). Plasmacytoid dendritic cells can be purified directly from peripheral blood on the basis of cell surface markers, and then incubated in the presence of IL-3 (Grouard et al., 1997, supra; Rissoan et al., 1999, supra). Alternatively, plasmacytoid DC can be derived from density gradients or CMRF-44 selection of incubated peripheral blood cells as above.

In general, for dendritic cells generated from any precursor, when incubated in the presence of activation factors such as monocyte-derived cytokines, lipopolysaccharide and DNA containing CpG repeats, cytokines such as TNF-α, IL-6, IFN-α, IL-1β, necrotic cells, re-adherence, whole bacteria, membrane components, RNA or polyIC, immature dendritic cells will become activated (Clark, 2002, J Leukoc Biol, 71, 388-400; Hacker et al., 2002, Immunology 105, 245-251; Kaisho and Akira, 2002, Biochim Biophys Acta 1589, 1-13; Koski et al., 2001, Crit Rev Immunol 21, 179-189). This process of dendritic cell activation is inhibited in the presence of NF-κB inhibitors (O'Sullivan and Thomas, 2002, J Immunol 168, 5491-5498).

In some embodiments, uncultured populations of antigen-presenting cells or their precursors can be introduced into the subject, which have not been subjected to activating conditions. Illustrative examples of the uncultured population of antigen-presenting cells or their precursors include whole blood, fresh blood, or fractions thereof such as but not limited to peripheral blood mononuclear cells (PMBC), buffy coat fractions of whole blood, packed red cells, irradiated blood, dendritic cells, monocytes, macrophages, neutrophils, lymphocytes, natural killer cells and natural killer T cells. In specific embodiments, the uncultured population of antigen-presenting cells is selected from freshly isolated blood or PMBC. In other embodiments, the uncultured population of antigen-presenting cells is a necrotic or apoptotic population. Thus, the uncultured population of cells may be contacted with antigen and subsequently subjected to necrotic conditions, which lead to irreversible trauma to cells (e.g., osmotic shock or exposure to chemical poison such as glutaraldehyde), wherein the cells are characterised by marked swelling of the mitochondria and cytoplasm, followed by cell destruction and autolysis. Alternatively, the uncultured cell population may be contacted with a bioactive molecule of the invention and subsequently subjected to apoptotic conditions. Cells expressing or presenting antigen can be induced to undergo apoptosis in vitro or in vivo using a variety of methods known in the art including, but not limited to, viral infection, irradiation with ultraviolet light, gamma radiation, steroids, fixing (e.g., with glutaraldehyde), cytokines or by depriving donor cells of nutrients in the cell culture medium. Time course studies can establish incubation periods sufficient for optimal induction of apoptosis in a population of cells. For example, monocytes infected with influenza virus begin to express early markers for apoptosis by 6 hours after infection. Examples of specific markers for apoptosis include Annexin V, TUNEL+ cells, DNA laddering and uptake of propidium iodide.

### 3.4.2 Ex vivo Delivery of Polypeptide or Nucleic Acid

Gag immune stimulators of the invention can be delivered into antigen-presenting cells in various forms, including nucleic acids and polypeptides, which may be soluble or particulate. The amount of Gag immune stimulator to be placed in contact with antigen-presenting cells can be determined empirically by persons of skill in the art. The antigen-presenting cells should be exposed to the Gag immune stimulator for a period of time sufficient for those cells to present Gag peptides on their surface for the modulation of T cells. In some advantageous embodiments the antigen-presenting cells are incubated in the presence of Gag polypeptide or Gag peptide for less than about 48, 36, 24, 12, 8, 7, 6, 5, 4, 3 or 2 hours or even for less that about 60, 50, 40, 30, 20, 15, 10, 9, 8, 7, 6, 5, 4, 3 or 2 minutes). The time and dose of polypeptide or peptides necessary for the cells to optionally process and present Gag peptides may be determined using pulse-chase protocols in which exposure to Gag antigen is followed by a washout period and exposure to a read-out system e.g., antigen reactive T cells. Once the optimal time and dose necessary for cells to express the Gag peptides on their surface is determined, a protocol may be used to prepare cells and Gag antigen for inducing immunogenic responses. Those of skill in the art will recognise in this regard that the length of time necessary for an antigen-presenting cell to present an antigen on its surface may vary depending on the antigen or form of antigen employed, its dose, and the antigen-presenting cell employed, as well as the conditions under which antigen loading is undertaken. These parameters can be determined by the skilled artisan using routine procedures. Efficiency of priming of the antigen-presenting cells can be determined by assaying T cell cytotoxic activity in vitro or using antigen-presenting cells as targets of CTLs. Other methods known to practitioners in the art, which can detect the presence of Gag peptide on the surface of antigen-presenting cells after exposure to a Gag antigen, are also contemplated by the presented invention.

Usually, about 0.1 to 20 μg/mL of Gag antigen (e.g., Gag peptide antigen) to about 1-10 million antigen-presenting cells is suitable for producing primed antigen-specific antigen-presenting cells. Typically antigen-presenting cells are incubated with antigen for about 1 to 6 hr at 37° C., although it is also possible to expose antigen-presenting cells to Gag antigen for the duration of incubation with one or more growth factors. As determined previously by the present inventors, successful presentation of peptide antigen can be achieved using much shorter periods of incubation (e.g., about 5, 10, 15, 20, 30, 40, 50 minutes) using antigen at a concentration of about 10-20 μg/mL.

If desired, all or a portion of the antigen-presenting cells can be frozen in an appropriate cryopreservative solution, until required. For example, the cells may be diluted in an appropriate medium, such as one containing 10% of autologous serum+10% of dimethylsulfoxide in a phosphate buffer saline. In certain embodiments, the cells are conserved in a dehydrated form.

In some embodiments, the delivery of exogenous Gag antigen to an antigen-presenting cell can be enhanced by methods known to practitioners in the art. For example, several different strategies have been developed for delivery of exogenous antigen to the endogenous processing pathway of antigen-presenting cells, especially dendritic cells. These methods include insertion of antigen into pH-sensitive liposomes (Zhou and Huang, 1994, Immunomethods 4, 229-235), osmotic lysis of pinosomes after pinocytic uptake of soluble antigen (Moore et al., 1988, Cell 54, 777-785), coupling of antigens to potent adjuvants (Aichele et al., 1990, J. Exp. Med., 171, 1815-1820; Gao et al., 1991, J. Immunol., 147, 3268-3273; Schulz et al., 1991, Proc. Natl. Acad. Sci. USA, 88, 991-993; Kuzu et al., 1993, Euro. J. Immunol. 23, 1397-1400; and Jondal et al., 1996, Immunity 5, 295-302), exosomes (Zitvogel et al., 1998 Nat Med. 4, 594-600; 2002, Nat Rev Immunol. 2, 569-79), and apoptotic cell delivery of antigen (Albert et al., 1998, Nature 392, 86-89; Albert et al., 1998, Nature Med. 4, 1321-1324; and in International Publications WO 99/42564 and WO 01/85207). Recombinant bacteria (eg. E. coli) or transfected host mammalian cells may be pulsed onto dendritic cells (as particulate antigen, or apoptotic bodies respectively) for antigen delivery. Such a delivery system might be logically combined with a substance for inhibiting NF-κB, such as a plasmid encoding dominant negative IκBα (Pai et al., 2002, J Virol 76, 1914-1921). Recombinant chimeric virus-like particles (VLPs) have also been used as vehicles for delivery of exogenous heterologous antigen to the MHC class I processing pathway of a dendritic cell line (Bachmann et al., 1996, Eur. J. Immunol., 26(11), 2595-2600).

Alternatively, or in addition, an antigen may be linked to, or otherwise associated with, a cytolysin to enhance the transfer of the antigen into the cytosol of an antigen-presenting cell of the invention for delivery to the MHC class I pathway. Exemplary cytolysins include saponin compounds such as saponin-containing Immune Stimulating Complexes (ISCOMs) (see e.g., Cox and Coulter, 1997, Vaccine 15(3), 248-256 and U.S. Pat. No. 6,352,697), phospholipases (see, e.g., Camilli et al., 1991, J. Exp. Med. 173, 751-754), pore-forming toxins (e.g., an alpha-toxin), natural cytolysins of gram-positive bacteria, such as listeriolysin O (LLO, e.g., Mengaud et al., 1988, Infect. Immun. 56, 766-772 and Portnoy et al., 1992, Infect. Immun. 60, 2710-2717), streptolysin O (SLO, e.g., Palmer et al., 1998, Biochemistry 37(8), 2378-2383) and perfringolysin O (PFO, e.g., Rossjohn et al., Cell 89(5), 685-692). Where the antigen-presenting cell is phagosomal, acid activated cytolysins may be advantageously used. For example, listeriolysin exhibits greater pore-forming ability at mildly acidic pH (the pH conditions within the phagosome), thereby facilitating delivery of vacuole (including phagosome and endosome) contents to the cytoplasm (see, e.g., Portnoy et al., 1992, Infect. Immun. 60, 2710-2717).

The cytolysin may be provided together with a Gag polypeptide or peptide in the form of a single composition or may be provided as a separate composition, for contacting the antigen-presenting cells. In one embodiment, the cytolysin is fused or otherwise linked to the Gag polypeptide or peptide, wherein the fusion or linkage permits the delivery of the Gag polypeptide or peptide to the cytosol of the antigen-presenting cell. In another embodiment, the cytolysin and Gag polypeptide or peptide are provided in the form of a delivery vehicle such as, but not limited to, a liposome or a microbial delivery vehicle selected from virus, bacterium, or yeast. Suitably, when the delivery vehicle is a microbial delivery vehicle, the delivery vehicle is non-virulent. In a preferred embodiment of this type, the delivery vehicle is a non-virulent bacterium, as for example described by Portnoy et al. in U.S. Pat. No. 6,287,556, comprising a first polynucleotide encoding a non-secreted functional cytolysin operably linked to a regulatory sequence which expresses the cytolysin in the bacterium, and a second polynucleotide encoding the Gag polypeptide or peptide. Non-secreted cytolysins may be provided by various mechanisms, e.g., absence of a functional signal sequence, a secretion incompetent microbe, such as microbes having genetic lesions (e.g., a functional signal sequence mutation), or poisoned microbes, etc. A wide variety of nonvirulent, non-pathogenic bacteria may be used; preferred microbes are relatively well characterised strains, particularly laboratory strains of E. coli, such as MC4100, MC1061, DH5α, etc. Other bacteria that can be engineered for the invention include well-characterised, nonvirulent, non-pathogenic strains of Listeria monocytogenes, Shigella flexneri, mycobacterium, Salmonella, Bacillus subtilis, etc. In a particular embodiment, the bacteria are attenuated to be non-replicative, non-integrative into the host cell genome, and/or non-motile inter- or intra-cellularly.

The delivery vehicles described above as well as the particulate vehicles described for example in Section 3.3 can be used to deliver one or more Gag polypeptides and/or peptides to virtually any antigen-presenting cell capable of endocytosis of the subject vehicle, including phagocytic and non-phagocytic antigen-presenting cells. In embodiments when the delivery vehicle is a microbe, the subject methods generally require microbial uptake by the target cell and subsequent lysis within the antigen-presenting cell vacuole (including phagosomes and endosomes).

### 4. Lymphocyte Embodiments

The antigen-presenting cells of the invention may be obtained or prepared to contain and/or express Gag antigens by any number of means, such that the antigen(s) or processed form(s) thereof, is (are) presented by those cells for potential modulation of other immune cells, including T lymphocytes and B lymphocytes, and particularly for producing T lymphocytes and B lymphocytes that are primed to respond to a Gag antigens. Lymphocytes that are primed to respond to Gag antigen are also referred to herein as Gag-primed lymphocytes.

In some embodiments, the Gag-specific antigen-presenting cells are useful for producing primed T lymphocytes to a Gag antigen. The efficiency of inducing lymphocytes, especially T lymphocytes, to exhibit an immune response to a Gag antigen can be determined by any suitable method including, but not limited to, assaying T lymphocyte cytolytic activity in vitro using for example antigen-specific antigen-presenting cells as targets of antigen-specific cytolytic T lymphocytes (CTL); assaying antigen-specific T lymphocyte proliferation (see, e.g., Vollenweider and Groseurth, 1992, J. Immunol. Meth. 149: 133-135), measuring B cell response to the antigen using, for example, ELISPOT assays, and ELISA assays; interrogating cytokine profiles; or measuring delayed-type hypersensitivity (DTH) responses by test of skin reactivity to a specified antigen (see, e.g., Chang et al. (1993, Cancer Res. 53: 1043-1050). Other methods known to practitioners in the art, which can detect the presence of Gag peptides on the surface of antigen-presenting cells after exposure to Gag antigen, are also contemplated by the present invention.

Accordingly, the present invention also provides antigen-specific B or T lymphocytes, especially T lymphocytes, which respond in an antigen-specific fashion to representation of a Gag antigen. In some embodiments, antigen-specific T lymphocytes are produced by contacting a Gag-specific antigen-presenting cell as defined above with a population of T lymphocytes, which may be obtained from any suitable source such as spleen or tonsil/lymph nodes but is preferably obtained from peripheral blood. The T lymphocytes can be used as crude preparations or as partially purified or substantially purified preparations, which are suitably obtained using standard techniques as, for example, described in “Immunochemical Techniques, Part G: Separation and Characterization of Lymphoid Cells” (Meth. in Enzymol. 108, Edited by Di Sabato et al., 1984, Academic Press). This includes rosetting with sheep red blood cells, passage across columns of nylon wool or plastic adherence to deplete adherent cells, immunomagnetic or flow cytometric selection using appropriate monoclonal antibodies is known in the art.

The preparation of T lymphocytes is contacted with the Gag-specific antigen-presenting cells of the invention for an adequate period of time for priming the T lymphocytes to the Gag antigen or antigens presented by those antigen-presenting cells. This period will preferably be at least about 1 day, and up to about 5 days.

In some embodiments, a population of Gag-specific antigen-presenting cells is cultured in the presence of a heterogeneous population of T lymphocytes, which is suitably obtained from peripheral blood, together with a plurality of Gag peptides of the invention. These cells are cultured for a period of time and under conditions sufficient for the peptides, or their processed forms, to be presented by the antigen-presenting cells; and for the antigen-presenting cells to prime a subpopulation of the T lymphocytes to respond to Gag antigen.

### 5. Cell Based Therapy or Prophylaxis

The Gag-specific antigen-presenting cells described in Section 3.4 and the Gag-primed lymphocytes described in Section 4 can be administered to a patient, either by themselves or in combination, for modulating an immune response, especially for modulating an immune response to a Gag polypeptide. These cell based compositions are useful, therefore, for treating or preventing a lentivirus infection or associated condition. The cells of the invention can be introduced into a patient by any means (e.g., injection), which produces the desired immune response to an antigen or group of antigens. The cells may be derived from the patient (i.e., autologous cells) or from an individual or individuals who are MHC matched or mismatched (i.e., allogeneic) with the patient. Typically, autologous cells are injected back into the patient from whom the source cells were obtained. The injection site may be subcutaneous, intraperitoneal, intramuscular, intradermal, intravenous or intralymphoid. The cells may be administered to a patient already suffering from a disease or condition or who is predisposed to a disease or condition in sufficient number to treat or prevent or alleviate the symptoms of the disease or condition. The number of cells injected into the patient in need of the treatment or prophylaxis may vary depending on inter alia, the antigen or antigens and size of the individual. This number may range for example between about 10³ and 10¹¹, and usually between about 10⁵ and 10⁷ cells (e.g., in the form blood, PMBC or purified dendritic cells or T lymphocytes). Single or multiple (2, 3, 4 or 5} administrations of the cells can be carried out with cell numbers and pattern being selected by the treating physician. The cells should be administered in a pharmaceutically acceptable carrier, which is non-toxic to the cells and the individual. Such carrier may be the growth medium in which the cells were grown, or any suitable buffering medium such as phosphate buffered saline. The cells may be administered alone or as an adjunct therapy in conjunction with other therapeutics known in the art for the treatment or prevention of unwanted immune responses for example but not limited to glucocorticoids, methotrexate, D-penicillamine, hydroxychloroquine, gold salts, sulfasalazine, TNF-alpha or interleukin-1 inhibitors, and/or other forms of specific immunotherapy.

### 6. Therapy and Prophylaxis

The Gag polypeptides and peptides described in Sections 3.1, and the Gag-expressing nucleic acid constructs described in Section 3.2, as well as the Gag particles described in Section 3.3, and the Gag-specific antigen-presenting cells described in Section 3.4 and the Gag-primed lymphocytes described in Section 4 (“immune stimulators” or “Gag immune stimulators”) can be used singly or together as active ingredients for the treatment or prophylaxis of lentiviral infections including the treatment of lentiviral-associated diseases or conditions such as but not limited to acquired immunodeficiency diseases. These immune stimulators can be administered to a patient either by themselves, or in compositions where they are mixed with a suitable pharmaceutically acceptable carrier and/or diluent, or an adjuvant.

Therefore, the invention encompasses methods for treating or preventing a lentivirus infection, which consist essentially of administering to a patient in need of such treatment an effective amount of at least one Gag immune stimulator as broadly described above. In some embodiments, the methods consist essentially of administering to an individual having a lentivirus infection, or at risk of having a lentivirus infection, a Gag immune stimulator in an amount effective to increase the number of Gag-specific antigen-presenting cells or their precursors to thereby treating or prevent the lentivirus infection.

Accordingly, the methods of the present invention are suitable for treating individuals who have a lentiviral infection; who are at risk of contracting a lentiviral infection; and who were treated for a lentiviral infection, but who relapsed. Such individuals include, but are not limited to, individuals with healthy, intact immune systems, but who are at risk for becoming HIV infected (“at-risk” individuals). At-risk individuals include, but are not limited to, individuals who have a greater likelihood than the general population of becoming HIV infected. Individuals at risk for becoming HIV infected include, but are not limited to, individuals at risk for HIV infection due to sexual activity with HIV-infected individuals; intravenous drug users; individuals who may have been exposed to HIV-infected blood, blood products, or other HIV-contaminated body fluids; and babies who are being nursed by HIV-infected mothers. Individuals suitable for treatment include individuals infected with, or at risk of becoming infected with, HIV-1 and/or HIV-2 and/or HIV-3, or any variant thereof Individuals suitable for treatment with the methods of the invention also include individuals who have a lentiviral infection that is refractory to treatment with other anti-viral therapies.

In specific embodiments, the methods are used to treat or prevent a lentivirus-associated disease (e.g., an acquired immunodeficiency disease such as AIDS) and thus the present invention also extends to methods of treating or preventing a lentivirus-associated disease in a subject, wherein the methods generally involve administering to the subject a Gag immune stimulator as broadly described above in an amount that is effective to treat or prevent the disease.

A Gag immune stimulator is administered to an individual using any available method and route suitable for drug delivery, including in vivo and ex vivo methods, as well as systemic and localized routes of administration. Techniques for formulation and administration may be found in “Remington's Pharmaceutical Sciences,” Mack Publishing Co., Easton, Pa., latest edition. Suitable routes may, for example, include enteral (e.g., oral, or rectal), transmucosal, or intestinal administration; parenteral delivery, including intramuscular, subcutaneous, intramedullary injections, as well as intrathecal, direct intraventricular, intravenous, intraperitoneal, intranasal, or intraocular injections. For injection, which constitutes one desirable embodiment of the present invention, the immune stimulators of the present invention may be formulated in aqueous solutions, typically in physiologically compatible buffers such as Hanks' solution, Ringer's solution, or physiological saline buffer. For transmucosal administration, penetrants appropriate to the barrier to be permeated are used in the formulation. Such penetrants are generally known in the art. Intra-muscular and subcutaneous injection is appropriate, for example, for administration of immunogenic compositions, vaccines and DNA vaccines. In certain embodiments of the present invention, the immune stimulators are administered intravenously.

The Gag immune stimulators can be formulated readily using pharmaceutically acceptable carriers well known in the art into dosages suitable for oral administration. Such carriers enable the compounds of the invention to be formulated in dosage forms such as tablets, pills, capsules, liquids, gels, syrups, slurries, suspensions and the like, for oral ingestion by a patient to be treated. These carriers may be selected from sugars, starches, cellulose and its derivatives, malt, gelatine, talc, calcium sulphate, vegetable oils, synthetic oils, polyols, alginic acid, phosphate buffered solutions, emulsifiers, isotonic saline, and pyrogen-free water.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as., for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Such compositions may be prepared by any of the methods of pharmacy but all methods include the step of bringing into association one or more therapeutic agents as described above with the carrier which constitutes one or more necessary ingredients. In general, the pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterise different combinations of active compound doses.

Pharmaceuticals which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilisers. In soft capsules, the active compounds may be dissolved or suspended in suitable liquids, such as fatty oils, liquid paraffin, or liquid polyethylene glycols. In addition, stabilisers may be added.

Dosage forms of the Gag immune stimulators of the invention may also include injecting or implanting controlled releasing devices designed specifically for this purpose or other forms of implants modified to act additionally in this fashion. Controlled release of an agent of the invention may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, controlled release may be effected by using other polymer matrices, liposomes and/or microspheres.

The Gag immune stimulators of the invention (e.g., Gag polypeptides and peptides) may be provided as salts with pharmaceutically compatible counterions. Pharmaceutically compatible salts may be formed with many acids, including but not limited to hydrochloric, sulphuric, acetic, lactic, tartaric, malic, succinic, etc. Salts tend to be more soluble in aqueous or other protonic solvents that are the corresponding free base forms.

Alternatively, one may administer a Gag immune stimulator in a local rather than systemic manner, for example, via injection of the compound directly into a tissue, often in a depot or sustained release formulation. Furthermore, one may administer the immune stimulator in a targeted drug delivery system, for example, in a liposome coated with tissue-specific antibody, as described for example in Section 3.3. The liposomes will be targeted to and taken up selectively by the tissue.

Pharmaceutical compositions suitable for use in the present invention include compositions wherein the active ingredients are contained in an effective amount to achieve its intended purpose. The dose of agent administered to a patient should be sufficient to effect a beneficial response in the patient over time such as a reduction in the symptoms associated with the condition. The quantity of the immune stimulator(s) to be administered may depend on the subject to be treated inclusive of the age, sex, weight and general health condition thereof. In this regard, precise amounts of the immune stimulator(s) for administration will depend on the judgement of the practitioner. In determining the effective amount of the immune stimulator to be administered in the treatment or prophylaxis of the condition, the physician may evaluate tissue levels of a target antigen, and progression of the disease or condition. In any event, those of skill in the art may readily determine suitable dosages of the immune stimulators of the invention.

For any compound used in the method of the invention, the effective dose can be estimated initially from cell culture assays or animal models. Toxicity and therapeutic efficacy of the immune stimulators of the invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration utilised. The exact formulation, route of administration and dosage can be chosen by the individual physician in view of the patient's condition. (See for example Fingl et al., 1975, in “The Pharmacological Basis of Therapeutics”, Ch. 1 p 1).

Dosage amount and interval may be adjusted individually to provide plasma levels of the active compound(s) which are sufficient to maintain Gag-reducing effects or effects that ameliorate the lentivirus infection or associated disease or condition. Usual patient dosages for systemic administration range from 1-2000 mg/day, commonly from 1-250 mg/day, and typically from 10-150 mg/day. Stated in terms of patient body weight, usual dosages range from 0.02-25 mg/kg/day, commonly from 0.02-3 mg/kg/day, typically from 0.2-1.5 mg/kg/day. Stated in terms of patient body surface areas, usual dosages range from 0.5-1200 mg/m²/day, commonly from 0.5-150 mg/m²/day, typically from 5-100 mg/m²/day.

In some embodiments, a single dose of a Gag immune stimulator is administered. In other embodiments, multiple doses of an immune stimulator are administered. Where multiple doses are administered over a period of time, an immune stimulator is administered twice daily (qid), daily (qd), every other day (qod), every third day, three times per week (tiw), or twice per week (biw) over a period of time. In illustrative examples, an immune stimulator is administered qid, qd, qod, tiw, or biw over a period of from one day to about 2 years or more. Suitably, an immune stimulator is administered at any of the aforementioned frequencies for one week, two weeks, one month, two months, six months, one year, or two years, or more, depending on various factors.

In some embodiments, an effective amount of an immune stimulator is one that reduces lentivirus load in a treated individual by at least about 10%, at least about 20%, at least about 30%, at least about 40%, at least about 50%, at least about 60%, at least about 70%, at least about 80%, at least about 90%, or at least about 95% or more, compared to the lentivirus load of the individual not treated with the immune stimulator.

In some embodiments, an effective amount of a Gag immune stimulator is one that increases the CD4⁺ T cell count in an individual by at least about 10%, at least about 20%, at least about 30%, at least about 40%, at least about 50%, at least about 60%, at least about 70%, at least about 80%, at least about 90%, at least about 100%, at least about 2.5-fold, at least about 3-fold, at least about 3.5-fold, at least about 4-fold, at least about 5-fold, or at least about 10-fold, or more, compared to the CD4⁺ T cell count of the individual not treated with the immune stimulator.

In some embodiments, an effective amount of a Gag immune stimulator is one that restores the CD4⁺ T cell count to within a normal range. In human blood, the number of CD4⁺-T cells which is considered to be in a normal range is from about 600 to about 1500 CD4⁺-T cells/mm³ blood.

Treating or preventing a lentivirus infection, includes, but is not limited to, reducing the probability of lentivirus infection, reducing the spread of lentivirus from an infected cell to a susceptible cell, reducing viral load in an lentivirus-infected individual, reducing an amount of virally-encoded polypeptide(s) in an lentivirus-infected individual, and increasing CD4⁺ T cell count in a lentivirus-infected individual.

Any of a variety of methods can be used to determine whether a treatment/prophylactic method is effective. For example, methods of determining whether the methods of the invention are effective in reducing lentivirus load, and/or treating an lentivirus infection, are any known test for indicia of lentivirus infection, including, but not limited to, measuring viral load, e.g., by measuring the amount of lentivirus in a biological sample, e.g., using a polymerase chain reaction (PCR) with primers specific for a lentivirus polynucleotide sequence; detecting and/or measuring a polypeptide encoded by lentivirus, e.g., p24, gp120, reverse transcriptase, using, e.g., an immunological assay such as an enzyme-linked immunosorbent assay (ELISA) with an antibody specific for the polypeptide; and measuring the CD4.sup.+T cell count in the individual. Methods of assaying a lentivirus infection (or any indicia associated with an lentivirus infection) are known in the art, and have been described in numerous publications such as HIV Protocols (Methods in Molecular Medicine, 17) N. L. Michael and J. H. Kim, eds. (1999) Humana Press.

From the foregoing, it will be appreciated that the agents of the invention may be used as therapeutic or prophylactic immunomodulating compositions or vaccines. Accordingly, the invention extends to the production of immunomodulating compositions containing as active compounds one or more of the Gag immune stimulators of the invention. Any suitable procedure is contemplated for producing such vaccines. Exemplary procedures include, for example, those described in New Generation Vaccines (1997, Levine et al., Marcel Dekker, Inc. New York, Basel Hong Kong).

Immunomodulating compositions according to the present invention can contain a physiologically acceptable diluent or excipient such as water, phosphate buffered saline and saline. They may also include an adjuvant as is well known in the art. Suitable adjuvants include, but are not limited to: surface active substances such as hexadecylamine, octadecylamine, octadecyl amino acid esters, lysolecithin, dimethyldioctadecylammonium bromide, N, N-dicoctadecyl-N′,N′bis(2-hydroxyethyl-propanediamine), methoxyhexadecylglycerol, and pluronic polyols; polyamines such as pyran, dextransulfate, poly IC carbopol; peptides such as muramyl dipeptide and derivatives, dimethylglycine, tuftsin; oil emulsions; and mineral gels such as aluminum phosphate, aluminum hydroxide or alum; lymphokines, QuilA and immune stimulating complexes (ISCOMS).

The Gag-specific antigen-presenting cells or precusors of the invention and Gag-primed T lymphocytes generated with the Gag-specific antigen-presenting cells, as described supra, can be used as immune stimulators in immunomodulating compositions for prophylactic or therapeutic applications. In some embodiments, the antigen-specific antigen-presenting cells of the invention are useful for generating large numbers of CD8⁺ or CD4+ CTL, for adoptive transfer to immunosuppressed individuals who are unable to mount normal immune responses. For example, Gag-primed CD8⁺ CTL can be adoptively transferred for therapeutic purposes in individuals afflicted with a lentiviral infection (Koup et al., 1991, J. Exp. Med., 174: 1593-1600; Carmichael et al., 1993, J. Exp. Med., 177: 249-256; and Johnson et al., 1992, J. Exp. Med., 175: 961-971).

### 7. Combination Therapies

A Gag immune stimulator can be administered to an individual in combination (e.g., in the same formulation or in separate formulations) with at least a second therapeutic agent (“combination therapy”). The immune stimulator can be administered in admixture with a second therapeutic agent or can be administered in a separate formulation. When administered in separate formulations, a Gag immune stimulator and a second therapeutic agent can be administered substantially simultaneously (e.g., within about 60 minutes, about 50 minutes, about 40 minutes, about 30 minutes, about 20 minutes, about 10 minutes, about 5 minutes, or about 1 minute of each other) or separated in time by about 1 hour, about 2 hours, about 4 hours, about 6 hours, about 10 hours, about 12 hours, about 24 hours, about 36 hours, or about 72 hours, or more. Effective amounts of a therapeutic agent are as described above.

Therapeutic agents that can be administered in combination therapy, such as anti-inflammatory, anti-viral, anti-fungal, anti-mycobacterial, antibiotic, amoebicidal, trichomonocidal, analgesic, anti-neoplastic, anti-hypertensives, anti-microbial and/or steroid drugs, to treat antiviral infections. In some embodiments, patients with a viral or bacterial infection are treated with a combination of one or more Gag immune stimulators with one or more of the following; beta-lactam antibiotics, tetracyclines, chloramphenicol, neomycin, gramicidin, bacitracin, sulfonamides, nitrofurazone, nalidixic acid, cortisone, hydrocortisone, betamethasone, dexamethasone, fluocortolone, prednisolone, triamcinolone, indomethacin, sulindac, acyclovir, amantadine, rimantadine, recombinant soluble CD4 (rsCD4), anti-receptor antibodies (e.g., for rhinoviruses), nevirapine, cidofovir (Vistide™), trisodium phosphonoformate (Foscamet™), famcyclovir, pencyclovir, valacyclovir, nucleic acid/replication inhibitors, interferon, zidovudine (AZT, Retrovir™), zidovudine/lamivudine (Combivir), didanosine (dideoxyinosine, ddI, Videx™), stavudine (d4T, Zerit™), zalcitabine (dideoxycytosine, ddC, Hivid™), nevirapine (Viramune™), lamivudine (Epivir™, 3TC), protease inhibitors, saquinavir (Invirase™, Fortovase™), ritonavir (Norvir™), nelfinavir (Viracept™), efavirenz (Sustiva™), abacavir (Ziagen™), amprenavir (Agenerase™) indinavir (Crixivan™), ganciclovir, AzDU, delavirdine (Rescriptor™), lopinavir/ritonavir (Kaletra), trizivir, rifampin, clathiromycin, erythropoietin, colony stimulating factors (G-CSF and GM-CSF), non-nucleoside reverse transcriptase inhibitors, nucleoside inhibitors, adriamycin, fluorouracil, methotrexate, asparaginase and combinations thereof. Anti-HIV agents are those in the preceding list that specifically target a function of one or more HIV proteins.

In some embodiments, a Gag immune stimulator is administered in combination therapy with two or more anti-HIV agents. For example, a subject agent can be administered in combination therapy with one, two, or three nucleoside reverse transcriptase inhibitors (e.g., Combivir, Epivir, Hivid, Retrovir, Videx, Zerit, Ziagen, etc.). An immune stimulator of the invention can be administered in combination therapy with one or two non-nucleoside reverse transcriptase inhibitors (e.g., Rescriptor, Sustiva, Viramune, etc.). A Gag immune stimulator can be administered in combination therapy with one or two protease inhibitors (e.g., Agenerase, Crixivan, Fortovase, Invirase, Kaletra, Norvir, Viracept, etc.). A Gag immune stimulator can be administered in combination therapy with a protease inhibitor and a nucleoside reverse transcriptase inhibitor. A Gag immune stimulator can be administered in combination therapy with a protease inhibitor, a nucleoside reverse transcriptase inhibitor, and a non-nucleoside reverse transcriptase inhibitor. A Gag immune stimulator can be administered in combination therapy with a protease inhibitor and a non-nucleoside reverse transcriptase inhibitor. Other combinations of a subject inhibitor with one or more of a protease inhibitor, a nucleoside reverse transcriptase inhibitor, and a non-nucleoside reverse transcriptase inhibitor are contemplated.

### 8. Methods for Assessing Immunomodulation

The effectiveness of an immunization may be assessed using any suitable technique. An individual's capacity to respond to Gag may be determined by assessing whether those cells primed to respond to Gag are increased in number, activity, and ability to detect and destroy that antigen or cells presenting that antigen. Strength of immune response is measured by standard tests including: direct measurement of peripheral blood lymphocytes by means known to the art; natural killer cell cytotoxicity assays (see, e.g., Provinciali M. et al (1992, J. Immunol. Meth. 155: 19-24), cell proliferation assays (see, e.g., Vollenweider, I. and Groseurth, P. J. (1992, J. Immunol. Meth. 149: 133-135), immunoassays of immune cells and subsets (see, e.g., Loeffler, D. A., et al. (1992, Cytom. 13: 169-174); Rivoltini, L., et al. (1992, Can. Immunol. Immunother. 34: 241-251); or skin tests for cell-mediated immunity (see, e.g., Chang, A. E. et al (1993, Cancer Res. 53: 1043-1050). Alternatively, the efficacy of the immunization may be monitored using one or more techniques including, but not limited to, HLA class I tetramer staining - of both fresh and stimulated PBMCs (see for example Allen et al., 2000, J. Immunol. 164(9): 4968-4978), proliferation assays (Allen et al., supra), ELISPOT assays and intracellular cytokine staining (Allen et al., supra), ELISA Assays—for linear B cell responses; and Western blots of cell sample expressing the synthetic polynucleotides. Particularly relevant will be the cytokine profile of T cells activated by antigen, and more particularly the production and secretion of IFN γ, IL-2, IL-4, IL-5, IL-10, TGFβ and TNF α.

The cytotoxic activity of T lymphocytes, and in particular the ability of cytotoxic T lymphocytes to be induced by antigen-presenting cells, may be assessed by any suitable technique known to those of skill in the art. For example, a sample comprising T lymphocytes to be assayed for cytotoxic activity is obtained and the T lymphocytes are then exposed to antigen-primed antigen-presenting cells, which have been caused to present antigen. After an appropriate period of time, which may be determined by assessing the cytotoxic activity of a control population of T lymphocytes which are known to be capable of being induced to become cytotoxic cells, the T lymphocytes to be assessed are tested for cytotoxic activity in a standard cytotoxic assay.

The method of assessing CTL activity is particularly useful for evaluating an individual's capacity to generate a cytotoxic response against cells expressing tumour or viral antigens. Accordingly, this method is useful for evaluating an individual's ability to mount an immune response to a cancer or virus. For example, CTL lysis assays may be employed using stimulated splenocytes or peripheral blood mononuclear cells (PBMC) on peptide coated or recombinant virus infected cells using ⁵¹Cr labelled target cells. Such assays can be performed using for example primate, mouse or human cells (Allen et al., supra). In addition, CTL activity can be measured in outbred primates using an in vivo detection method, which involves labeling autologous cells (e.g., PMBC) with an optically detectable label (e.g., a fluorescent, chemiluminescent or phosphorescent or visual label or dye) and contacting them with one ore more Gag peptides as disclosed herein. The are chosen so that they correspond to an antigen which is the subject of a CTL response under test in a subject.

The autologous cells are infused into the subject and lymphocytes from the subject are harvested after a suitable period to permit the subject's immune system sufficient time to respond to the autologous cells (e.g., 10 minutes to 24 hours post infusion). The harvested lymphocytes are then analysed to identify the number or proportion of lymphocytes which contain or otherwise carry the optically detectable label, which represents a measure of the in vivo CTL response to the antigen in the subject.

In order that the invention may be readily understood and put into practical effect, particular preferred embodiments will now be described by way of the following non-limiting examples.

### Experimental

Control of Viremia following Immunotherapy of SIV-Infected Macaques with Peptide Pulsed Blood

OPAL immunotherapy was studied in SIV-infected pigtail macaques receiving ART. Pigtail macaques have at least an equivalently pathogenic course of SIV infection than alternate rhesus macaque models^{9, 10}. Thirty-six macaques were infected with SIV_{mac251} and 3 weeks later treatment with the antiretrovirals tenofovir and emtricitabine for 7 weeks was initiated. The animals were randomly allocated to 3 groups stratified by peak plasma SW viral load (VL), Mane-A*10 status (an MHC class I gene that improves VL in SIV-infected pigtail macaques¹¹), weight and gender. Macaques were immunized 4 times under the cover of antiretroviral therapy (weeks 4, 6, 8, 10) with autologous fresh PBMC mixed for 1 hour ex vivo with 10 μg/mL/peptide of either 125 overlapping SIV Gag 15 mer peptides only (OPAL-Gag), 823 SIV 15 mer peptides spanning all 9 SIV proteins (OPAL-All) or unimmunized. The macaques were followed for 26 weeks after ceasing ART on week 10.

All 36 macaques became infected following _{SIVmac251} exposure and had a mean peak VL of 7.1 log₁₀ copies/mL. Prior to vaccination, 4 animals died during acute SIV infection with diarrhoea, dehydration, lethargy, anorexia and weight loss. The vaccinations were well tolerated, with no differences in mean weights, haematology parameters, or clinical observations in OPAL immunized animals compared to controls.

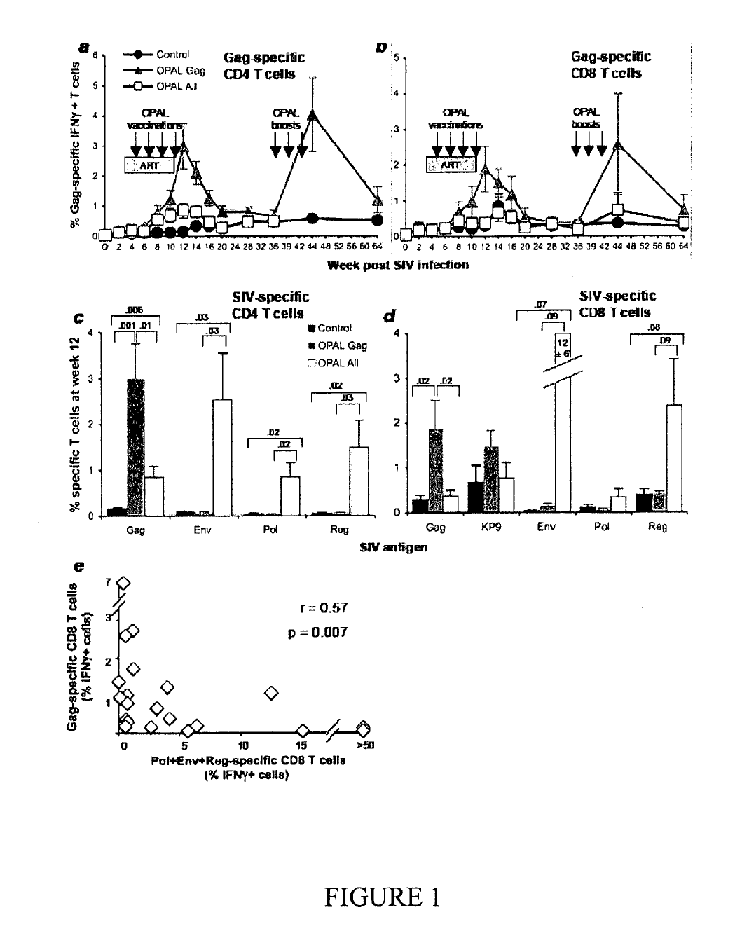

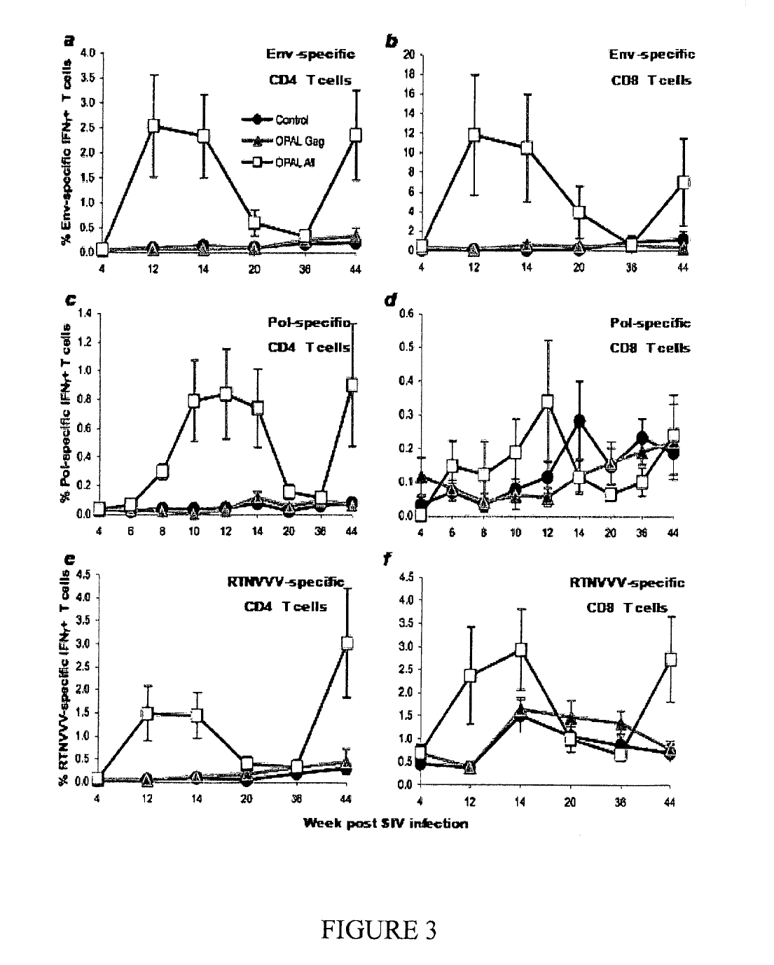

There was striking SIV-specific CD4⁺ and CD8⁺ T-cell immunogenicity after the course of vaccination in the OPAL immunized animals. Mean Gag-specific CD4 and CD8 T-cell responses 2 weeks after the final immunization were 3.0% and 1.9% of all CD4 and CD8 T cells respectively in the OPAL-Gag group. Mean Gag-specific CD4 and CD8 T-cell responses 2 weeks after the final immunization were 0.84% and 0.37% in the OPAL-All group and 0.15% and 0.29% in controls (FIG. 1 a,b). The Gag-specific T cells in the OPAL-All immunized animals, but not control or OPAL-Gag only immunized animals, also had elevated T-cell responses to all other SIV proteins. Mean Env, Pol and combined regulatory protein-specific CD4/CD8 responses were 2.5%/11.8%, 0.8%/0.3% and 1.5%/2.4%, respectively, in the OPAL-All group compared to ≦0.4% for all CD4/8 responses to non-Gag antigens in control and OPAL-Gag groups (FIG. 1c,d and FIG. 3). Stronger CD8⁺ T-cell responses to non-Gag proteins correlated with reduced CD8⁺ T-cell responses to Gag (FIG. 1e). Thus, although a larger number of SIV proteins were recognized in the OPAL-All immunized animals, Gag responses were reduced in comparison to only immunizing with Gag peptides. Although all animals seroconverted following SIV infection, no significant enhancement of antibody responses occurred with the OPAL vaccinations.

The 7-week period of ART controlled VL to below 3.1 log10 copies/mL in 26 of the remaining 32 animals by week 10. The 6 animals that failed to control viremia on ART had higher peak VLs at week 2 (mean±SD of 7.74±0.33 compared to 6.94±0.52 for animals controlling viremia on ART, p<0.001) and higher VL following ART withdrawal (5.98±0.53 vs 4.28±0.90, p<0.001). Control of VL is likely to be important in achieving optimal results from immunotherapy of infected macaques 7, 12. The pre-defined (per-protocol) primary VL endpoint analyses was performed on animals controlling viremia on ART (26 animals), although the inventors also analysed all 32 remaining animals by adjusting for VL control on ART.

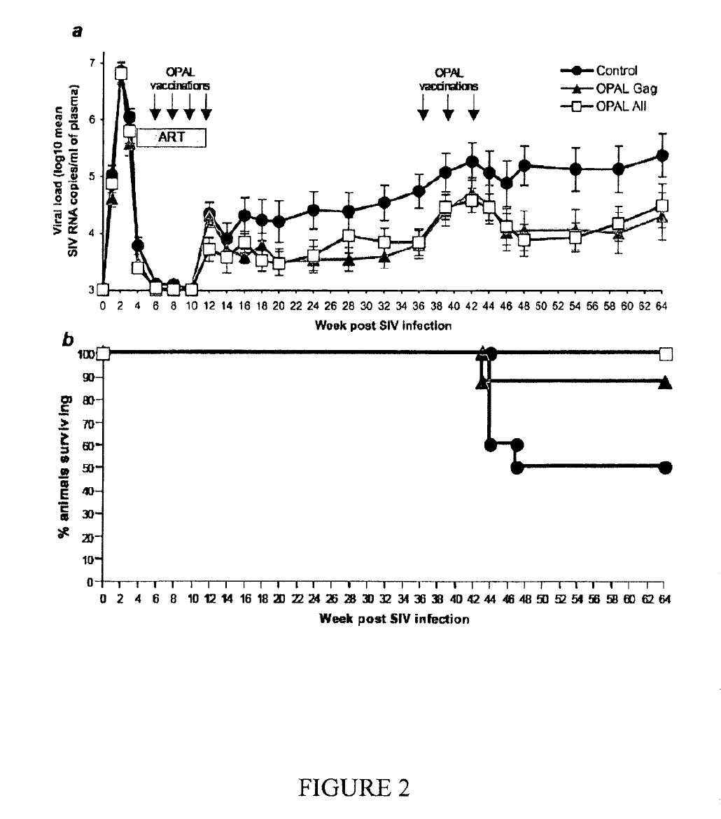

The primary endpoint comparison of VL between combined OPAL-All and OPAL-Gag treatment groups in the 10 weeks after ART withdrawal was 0.5 log₁₀ copies/mL lower than controls (p=0.084, FIG. 2, Table 7). Each vaccination group (OPAL-All and OPAL-Gag) had very similar reductions in VL. Analysis of all animals adjusted for control of VL on ART and Mane-A*10 status demonstrated a significant reduction in VL in OPAL-immunized animals compared to controls (Table 7). By 6 months after ART withdrawal, the mean difference in VL between control and OPAL-immunized groups was 0.93 log₁₀ copies/mL 6 months (p=0.02, Table 7).

To confirm the virologic findings using a sensitive independent VL assay, frozen plasma (1 mL) from study week 32 was shipped to the National Cancer Institute (NCI) in Maryland, USA. Drs M Piatak and J Lifson kindly analyzed the samples for SIV RNA blindly using an assay with a limit of quantitation of 1.5 logᵢₒ copies/mL The University of Melbourne and NCI assays were tightly correlated (r=0.97, p<0.001) and showed an almost identical mean reduction in viremia in vaccinees compared to controls at this time (0.82 vs 0.88 log10 copies/mL, respectively).

To further assess the durability of SIV control and prevention of disease with OPAL immunotherapy, we re-boosted all 32 animals in the same randomised groups 3 times with the identical procedure (at week 36, 39, 42) without ART cover and followed the animals for an additional 6 months. SIV-specific T cell immunity was enhanced in immunized animals similarly to the primary vaccination (FIG. 1). Viral control was maintained throughout the follow up period of just over 1 year off ART (FIG. 2, Table 7).

Twelve of the remaining 32 animals developed incipient AIDS and were euthanized during the extended follow up, including all 6 animals that did not control viremia on ART. Of the 6 euthanized animals which did control viremia on ART, 5 were in the control group and one in the OPAL-Gag group (FIG. 2). OPAL immunotherapy resulted in a survival benefit, analyzing either the 26 animals that controlled viremia on ART (p=0.053) or all 32 animals, adjusted for Mane-A*10 status and control of viremia on ART (p=0.02, Table 7).

The inventorsalso compared VL and peripheral CD4 levels in Env and Gag CTL responders. Primary analyses were conducted on animals within the vaccine groups to assess the effect of enhancing Env- or Gag-specific T-cells by therapeutic immunization. Mane-A*10 positive animals were excluded, given these animals all mount beneficial Gag-specific CTL responses to the KP9 epitope. Indeed, the ManeA*10 positive controls in this trial had an average VL through weeks 12-64 post infection of 4.06±0.42 log₁₀ copies/mL compared with 5.49±0.35 logᵢₒ copies/mL for ManeA*10 negative controls (P=0.024, time-weighted area-under-the curve analysis).

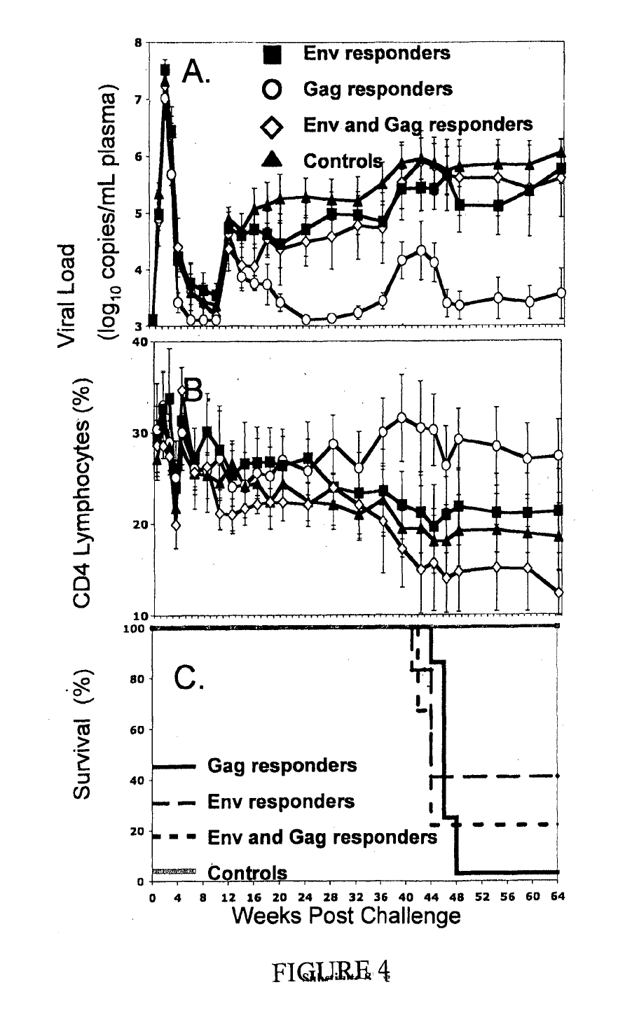

Env-only CTL responders maintained a significantly higher average VL between weeks 12-64 post infection than did animals with Gag-only CD8⁺ T-cell responses excluding the Mane-A*10+ animals (5.05±0.38 log₁₀ copies/mL vs 3.65±0.24 log₁₀ copies/mL respectively. P=0.039, FIG. 4). There was difference between average VL of the 6 Env-only responders between weeks 12-64 post infection and the 7 Mane-A*10 negative, unvaccinated, control animals (5.05±0.38 log₁₀ copies/mL and 5.49±0.35 log₁₀ copies/mL, respectively; FIG. 4).

To address speculation that a broad, multi-protein response could be beneficial, those animals with CD8⁺ T-cell responses to both Env and Gag were then included in this analysis. VL for the 3 animals with both Env- and Gag-specific responses averaged 5.01 35 0.56 log₁₀ copies/mL between weeks 12-64 post infection. This was significantly higher than the Gag-only responders (P=0.049) and no better than the Env-only response.

The animals in this trial were followed for just over 1 year after the last vaccination and removal of ART. This enabled an analysis of peripheral CD4⁺ T-cell depletion and survival in animals responding to only Env or Gag, those responding to both Env and Gag and the unvaccinated controls. There were non significantly higher average peripheral CD4 levels in the 3 Gag-only responders verses the 6 Env-only responders between weeks 12-64 post infection (23.42%±4.22 and 27.44%±3.92 respectively, P=0.547 FIG. 4). The 3 animals that had both Env- and Gag-specific CD8⁺ T-cell responses had a non significant but lower average peripheral CD4 level over the same time period than did the unvaccinated controls (18.59%±3.23 and 21.19%±3.01, respectively; FIG. 4).

During follow up, a total of 6 vaccinated animals and 6 controls of the 32 animals were euthanized with incipient AIDS, including weight loss, CD4⁺ T-cell depletion and thrombocytopenia. Animals responding to Env-only CD8⁺ T-cell epitopes progressed to AIDS more frequently than animals with CTL responses to Gag alone (FIG. 4).

In summary, OPAL immunotherapy, either using overlapping Gag SIV peptides or peptides spanning the whole SIV proteome was highly immunogenic and resulted in significantly lower viral loads and a survival benefit compared to unvaccinated controls. The virologic efficacy in OPAL-immunized macaques was durable for 12 months after ART cessation. The present findings on OPAL immunotherapy were observed despite the virulent SIV_{mac251}-pigtail model studied⁹ and provide strong proof-of-principle for the promise of this immunotherapy technique.

The OPAL immunotherapy approach is simpler than many other cellular immunotherapies, particularly the use of dendritic cells. The use of DNA, CTLA-4 blockade and viral vector based approaches are also now showing some promise in macaque studies^{14, 15}, although such approaches have not yet been translated into human studies. This study added peptides to PBMC, however the present inventors have shown an even simpler technique, adding peptides to whole blood is also highly immunogenic, a technique that will be more widely applicable⁷.

Virus-specific CD4⁺ T cells are typically very weak in HIV-infected humans or SIV-infected macaques; dramatic enhancement of these cells were induced by OPAL immunotherapy and this may underlie its efficacy¹⁶. Although the present inventors primarily measured IFN-γ-producing T cells in this study, recent polyfunctional ICS assays suggests OPAL immunotherapy can also induce T cells capable of also expressing the cytokines TNF-α and IL-2, the chemokine MIP1β and the degranulation marker CD 107a. Both the control and vaccinated macaques were treated with ART early (3 weeks after infection), which alone is associated with transiently improved outcome in humans. Nonetheless, a massive loss of CD4⁺ T cells in the gut within 2 weeks of infection¹⁷ and, although it may be challenging to identify humans this early after infection, it is around the time HIV-1 subjects present with acute infection. A ˜1.0 log₁₀ reduction in VL would result in a substantial delay in progressive HIV disease in humans and allow a reasonable time period without the requirement to reintroduce ART¹⁸ if these findings are confirmed in human trials. The durable control of viremia exhibited by the vaccinated animals is interesting and consistent with other recent macaque studies¹⁴, suggesting the need for re-immunization may not be substantial.

Control of viremia was similar for the OPAL-Gag and OPAL-All groups. Gag-specific CD4 and CD8⁺ T-cell responses in OPAL-Gag animals 5.1- and 3.5-fold greater than those in the OPAL-All animals, despite an identical dose of Gag overlapping peptides. This suggests antigenic competition between peptides from Gag and the other SIV proteins. Env-specific CD8 T-cell responses identified were unable to significantly impact viral replication or disease progression. Env (or other non-Gag) responses may potentially inhibit more effective CD8⁺ T-cell responses. This phenomenon is highlighted by the observation that a Gag-specific CTL response correlated with control of viremia, yet animals with both Env- and Gag-specific CD8⁺ T-cell responses fared no better than Env-only responders or unvaccinated controls, in both controlling viremia and preventing disease. Inducing immunodominant non-Gag T-cell responses by multi-protein HIV vaccines may limit the development of Gag-specific T-cell responses¹⁹. A large human cohort study demonstrated Gag-specific T cell responses were the most effective in controlling HIV viremia²⁰. Taken together, these studies suggest that therapeutic HIV vaccines may not need to aim for maximally broad multi-protein HIV-specific immunity. OPAL immunotherapy with Gag peptides is proceeding into initial trials in HIV-infected humans.

### Materials and Methods

### Animals

Juvenile pigtail macaques (Macaca nemestrina) free from Simian retrovirus type D were studied in protocols approved by institutional animal ethics committees and cared for in accordance with Australian National Health and Medical Research Council guidelines. All pigtail macaques were typed for MHC class I alleles by reference strand mediated conformational analysis and the presence of Mane-A*10 confirmed by sequence specific primer PCR as described^{21, 22, 36} macaques were injected intravenously with 40 tissue culture infectious doses of SIV_{mac251} (kindly provided by R. Pal, Advanced Biosciences, Kensington, Md.) as described previously^{9, 11} and randomized into 3 groups of 12 animals (OPAL-Gag, OPAL-All, Controls) 3 weeks later. Randomization was stratified for peak SIV viral load at week 2, weight, gender and the MHC I gene Mane-A*10 (which is known to enhance immune control of SIV)¹¹. Animals received subcutaneous injections of dual antiretroviral therapy with tenofovir and emtricitibine (kindly donated by Gilead, Foster City, Calif.; both 30mg/kg/animal) for 7 weeks from week 3: daily from weeks 3-5 postinfection and three times per week from weeks 6-10. This dual ART controls viremia in the majority of SIV-infected macaques^{12, 15, 23-25}.

### Immunizations

Two animal groups (OPAL-Gag and OPAL-All) were immunized with OPAL immunotherapy using PBMC as previously described. Briefly, peripheral blood mononuclear cells (PBMC) were isolated over Ficoll-paque from 18 mL of blood (anticoagulated with Na⁺-Heparin). All isolated PBMC (on average 24 million cells) were suspended in 0.5 mL of normal saline to which either a pool of 125 SIV_{mac239} Gag peptides or 823 peptides spanning all SIVmac239 proteins (Gag, Pol, Env, Nef, Vif, Tat, Rev, Vpr, Vpx) were added at 10 μg/mL of each peptide within the pool. Peptides were 15-mers overlapping by 11 amino acids at >80% purity kindly provided by the NIH AIDS reagent repository program (catalog #'s 6204, 6443, 6883, 6448-50, 6407, 8762, 6205). To pool the peptides, each 1 mg vial of lyophilized 15 mer peptide was solubilized in 10-50 μL of pure DMSO and added together. The concentration of the SIV Gag and All peptide pools was 629 and 72 μg/mL/peptide respectively. The peptide-pulsed PBMC were held for 1 hr in a 37° C. water bath, gently vortexed every 15 minutes and then, without washing, reinfused IV into the autologous animal. Control macaques did not receive vaccine treatment.

### Immunology Assays

SIV-specific CD4 and CD8 T-cell immune responses were analysed by expression of intracellular IFN-y as previously described¹⁹. Briefly, 200 μL whole blood was incubated at 37° C. with 1 μg/mL/peptide overlapping 15 mer SIV peptide pools (described above) or DMSO alone and the co-stimulatory antibodies anti-CD28 and anti-CD49d (BD Biosciences/Pharmingen San Diego Calif.) and Brefeldin A (10 μg/mL, Sigma) for 6 hr. Anti-CD3-PE, anti-CD4-FITC and anti-CD8-PerCP (BD, clones SP34, M-T477 and SK1 respectively) antibodies were added for 30 minutes. Red blood cells were lysed (FACS lysing solution, BD) and the remaining leukocytes permeabilized (FACS Permeabilizing Solution 2, BD) and incubated with anti-human IFN-γ-APC antibody (BD, clone B27) prior to fixation and acquisition (LSRII, BD). Acquisition data were analyzed using Flowjo version 6.3.2 (Tree Star, Ashland, Oreg.). The percentage of antigen-specific gated lymphocytes expressing IFNγ was assessed in both CD3⁺CD4⁺ and CD3⁺CD8⁺ lymphocyte subsets. Responses to the immunodominant SIV Gag CD8 T-cell epitope KP9 in Mane-A*10⁺ animals were assessed by a Mane-A*10/KP9 tetramer as described²². Total peripheral CD4 T-cells were measured as a proportion of lymphocytes by flow cytometry on fresh blood.

### Virology Assays

Plasma SIV RNA was quantitated by real time PCR on 140 μL of plasma at the University of Melbourne (lower limit of quantitation 3.1 log₁₀ copies/mL) at all time points using a TaqMan® probe as previously described^{19, 26} and, to validate these results with a more sensitive assay, on pelleted virions from 1 0 mL of plasma at the National Cancer Institute (lower limit of quantitation 1.5 log₁₀ copies/mL) as previously described¹³. To identify whether mutational escape occurred at the KP9 epitope we performed RT-PCR cloning and sequencing of extracted plasma viral cDNA across KP9 in Gag as previously described²⁷.

### Endpoints/Statistical Analyses

The primary endpoint was the reduction in plasma SIV RNA in OPAL-immunized animals compared to controls by time-weighted area-under-the-curve (TWAUC) for 10 weeks following withdrawal of ART (i.e. samples from weeks 12 to 20). This summary statistical approach is recommended for studies such as these involving serial measurements²⁸. The inventors compared both active treatment groups (OPAL-Gag and OPAL-All) to controls separately and together. The primary analysis was restricted to animals that controlled viremia on the ART at week 10 (VL<3.1 log10 copies/mL), since control of VL is an important predictor of the ability of animals to respond to in⁻imunotherapies^{7, 29}. A pre-planned secondary virologic endpoint was studying all live animals adjusting for both VL at the end of ART (week 10) and Mane-A*10 status. Group comparisons used two-sample t-tests for continuous data, and Fisher's exact test for binary data. Survival analyses utilised Cox-regression analyses.

### Power Calculation

The present inventors estimated the standard deviation of the return of VL after treatment interruption would be 0.8 log10 copies of SIV RNA/mL plasma^{5, 12, 15, 23-25}. In this intensive study, it was estimated that 2 of the 12 monkeys within a group may have confounding problems such as incomplete response to ART or death from acute SIV infection. A 10 control vs 10 active treatment comparison yields 80% power (p=0.05) to detect a 1.0 log10 difference in TWAUC VL over the first 10 weeks. An estimated comparison of 10 control vs all 20 actively treated animals (OPAL-Gag plus OPAL-All) gave 80% power to detect differences of 0.87 log10 copies/mL VL reduction.

### Study Conduct

This study was conducted according to a pre-written protocol using Good Laboratory Practice Standards from the Australian Therapeutic Goods Administration as a guide. Protocol deviations were minor and did not affect the results of the study. Partial data audits during the study did not raise any concerns about the study conduct.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### Tables

TABLE 1
CONSERVATIVE AMINO ACID SUBSTITUTIONS

| | Exemplary |
|---|---|
| Orginal Residue | Substitutions |
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile, |
| Phe | Met, Leu, Tyr |
| Ser | Thr |
| Thr | Ser |
| Trp | Tyr |
| Tyr | Trp, Phe |
| Val | Ile, Leu |

TABLE 2
One embodiment of an SIV_{mac236} gag peptide pool
sequence. Each peptide is 15 amino acids in
length and overlaps the preceding peptide by 11
amino acids. Peptide 125 is 14 amino acids in
length. The full-length gag sequence
[SEQ ID NO: 250] is modified from the HIV
sequence database http://hiv-web.lanl.gov.

| # | PEPTIDE | SEQUENCE ID |
|---|---|---|
| 1 | MGVRNSVLSGKKADE | SEQ ID NO: 1 |
| 2 | NSVLSGKKADELEKI | SEQ ID NO: 2 |
| 3 | SGKKADELEKIRLRP | SEQ ID NO: 3 |
| 4 | ADELEKIRLRPNGKK | SEQ ID NO: 4 |
| 5 | EKIRLRPNGKKKYML | SEQ ID NO: 5 |
| 6 | LRPNGKKKYMLKHVV | SEQ ID NO: 6 |
| 7 | GKKKYMLKHVVWAAN | SEQ ID NO: 7 |
| 8 | YMLKHVVWAANELDR | SEQ ID NO: 8 |
| 9 | HVVWAANELDRFGLA | SEQ ID NO: 9 |
| 10 | AANELDRFGLAESLL | SEQ ID NO: 10 |
| 11 | LDRFGLAESLLENKE | SEQ ID NO: 11 |
| 12 | GLAESLLENKEGCQK | SEQ ID NO: 12 |
| 13 | SLLENKEGCQKILSV | SEQ ID NO: 13 |
| 14 | NKEGCQKILSVLAPL | SEQ ID NO: 14 |
| 15 | CQKILSVLAPLVPTG | SEQ ID NO: 15 |
| 16 | LSVLAPLVPTGSENL | SEQ ID NO: 16 |
| 17 | LSVLAPLVPTGSENL | SEQ ID NO: 17 |
| 18 | PTGSENLKSLYNTVC | SEQ ID NO: 18 |
| 19 | ENLKSLYNTVCVIWC | SEQ ID NO: 19 |
| 20 | SLYNTVCVIWCIHAE | SEQ ID NO: 20 |
| 21 | TVCVIWCIHAEEKVK | SEQ ID NO: 21 |
| 22 | IWCIHAEEKVKHTEE | SEQ ID NO: 22 |
| 23 | HAEEKVKHTEEAKQI | SEQ ID NO: 23 |
| 24 | KVKHTEEAKQIVQRH | SEQ ID NO: 24 |
| 25 | TEEAKQIVQRHLVVE | SEQ ID NO: 25 |
| 26 | KQIVQRHLVVETGTT | SEQ ID NO: 26 |
| 27 | QRHLVVETGTTETMP | SEQ ID NO: 27 |
| 28 | VVETGTTETMPKTSR | SEQ ID NO: 28 |
| 29 | GTTETMPKTSRPTAP | SEQ ID NO: 29 |
| 30 | TMPKTSRPTAPSSGR | SEQ ID NO: 30 |
| 31 | TSRPTAPSSGRGGNY | SEQ ID NO: 31 |
| 32 | TAPSSGRGGNYPVQQ | SEQ ID NO: 32 |
| 33 | SGRGGNYPVQQIGGN | SEQ ID NO: 33 |
| 34 | GNYPVQQIGGNYVHL | SEQ ID NO: 34 |
| 35 | VQQIGGNYVHLPLSP | SEQ ID NO: 35 |
| 36 | GGNYVHLPLSPRTLN | SEQ ID NO: 36 |
| 37 | VHLPLSPRTLNAWVK | SEQ ID NO: 37 |
| 38 | LSPRTLNAWVKLIEE | SEQ ID NO: 38 |
| 39 | TLNAWVKLIEEKKFG | SEQ ID N0: 39 |
| 40 | WVKLIEEKKFGAEVV | SEQ ID NO: 40 |
| 41 | IEEKKFGAEVVPGFQ | SEQ ID NO: 41 |
| 42 | KFGAEVVPGFQALSE | SEQ ID NO: 42 |
| 43 | EVVPGFQALSEGCTP | SEQ ID NO: 43 |
| 44 | GFQALSEGCTPYDIN | SEQ ID NO: 44 |
| 45 | LSEGCTPYDINQMLN | SEQ ID NO: 45 |
| 46 | CTPYDINQMLNCVGD | SEQ ID NO: 46 |
| 47 | DINQMLNCVGDHQAA | SEQ ID NO: 47 |
| 48 | MLNCVGDHQAAMQII | SEQ ID NO: 48 |
| 49 | VGDHQAAMQIIRDII | SEQ ID NO: 49 |
| 50 | QAAMQIIRDIINEEA | SEQ ID NO: 50 |
| 51 | QIIRDIINEEAADWD | SEQ ID NO: 51 |
| 52 | DIINEEAADWDLQHP | SEQ ID NO: 52 |
| 53 | EEAADWDLQHPQPAP | SEQ ID NO: 53 |
| 54 | DWDLQHPQPAPQQGQ | SEQ ID NO: 54 |
| 55 | QHPQPAPQQGQLREP | SEQ ID NO: 55 |
| 56 | PAPQQGQLREPSGSD | SEQ ID NO: 56 |
| 57 | QGQLREPSGSDIAGT | SEQ ID NO: 57 |
| 58 | REPSGSDIAGTTSSV | SEQ ID NO: 58 |
| 59 | GSDIAGTTSSVDEQI | SEQ ID NO: 59 |
| 60 | AGTTSSVDEQIQWMY | SEQ ID NO: 60 |
| 61 | SSVDEQIQWMYRQQN | SEQ ID N0: 61 |
| 62 | EQIQWMYRQQNPIPV | SEQ ID NO: 62 |
| 63 | WMYRQQNPIPVGNIY | SEQ ID NO: 63 |
| 64 | QQNPIPVGNIYRRWI | SEQ ID NO: 64 |
| 65 | IPVGNIYRRWIQLGL | SEQ ID NO: 65 |
| 66 | NIYRRWIQLGLQKCV | SEQ ID NO: 66 |
| 67 | RWIQLGLQKCVRMYN | SEQ ID NO: 67 |
| 68 | LGLQKCVRMYNPTNI | SEQ ID NO: 68 |
| 69 | KCVRMYNPTNILDVK | SEQ ID NO: 69 |
| 70 | MYNPTNILDVKQGPK | SEQ ID NO: 70 |
| 71 | TNILDVKQGPKEPFQ | SEQ ID NO: 71 |
| 72 | DVKQGPKEPFQSYVD | SEQ ID NO: 72 |
| 73 | GPKEPFQSYVDRFYK | SEQ ID NO: 73 |
| 74 | PFQSYVDRFYKSLRA | SEQ ID NO: 74 |
| 75 | YVDRFYKSLRAEQTD | SEQ ID NO: 75 |
| 76 | FYKSLRAEQTDAAVK | SEQ ID NO: 76 |
| 77 | LRAEQTDAAVKNWMT | SEQ ID NO: 77 |
| 78 | QTDAAVKNWMTQTLL | SEQ ID NO: 78 |
| 79 | AVKNWMTQTLLIQNA | SEQ ID NO: 79 |
| 80 | WMTQTLLIQNANPDC | SEQ ID NO: 80 |
| 81 | TLLIQNANPDCKLVL | SEQ ID NO: 81 |
| 82 | QNANPDCKLVLKGLG | SEQ ID NO: 82 |
| 83 | PDCKLVLKGLGVNPT | SEQ ID NO: 83 |
| 84 | LVLKGLGVNPTLEEM | SEQ ID NO: 84 |
| 85 | GLGVNPTLEEMLTAC | SEQ ID NO: 85 |
| 86 | NPTLEEMLTACQGVG | SEQ ID NO: 86 |
| 87 | EEMLTACQGVGGPGQ | SEQ ID NO: 87 |
| 88 | TACQGVGGPGQKARL | SEQ ID NO: 88 |
| 89 | GVGGPGQKARLMAEA | SEQ ID NO: 89 |
| 90 | PGQKARLMAEALKEA | SEQ ID NO: 90 |
| 91 | ARLMAEALKEALAPV | SEQ ID NO: 91 |
| 92 | AEALKEALAPVPIPF | SEQ ID NO: 92 |
| 93 | KEALAPVPIPFAAAQ | SEQ ID NO: 93 |
| 94 | APVPIPFAAAQQRGP | SEQ ID NO: 94 |
| 95 | IPFAAAQQRGPRKPI | SEQ ID NO: 95 |
| 96 | AAQQRGPRKPIKCWN | SEQ ID NO: 96 |
| 97 | RGPRKPIKCWNCGKE | SEQ ID NO: 97 |
| 98 | KPIKCWNCGKEGHSA | SEQ ID NO: 98 |
| 99 | CWNCGKEGHSARQCR | SEQ ID NO: 99 |
| 100 | GKEGHSARQCRAPRR | SEQ ID NO: 100 |
| 101 | HSARQCRAPRRQGCW | SEQ ID NO: 101 |
| 102 | QCRAPRRQGCWKCGK | SEQ ID NO: 102 |
| 103 | PRRQGCWKCGKMDHV | SEQ ID NO: 103 |
| 104 | GCWKCGKMDHVMAKC | SEQ ID NO: 104 |
| 105 | CGKMDHVMAKCPDRQ | SEQ ID NO: 105 |
| 106 | DHVMAKCPDRQAGFL | SEQ ID NO: 106 |
| 107 | AKCPDRQAGFLGLGP | SEQ ID NO: 107 |
| 108 | DRQAGFLGLGPWGKK | SEQ ID NO: 108 |
| 109 | GFLGLGPWGKKPRNF | SEQ ID NO: 109 |
| 110 | LGPWGKKPRNFPMAQ | SEQ ID NO: 110 |
| 111 | GKKPRNFPMAQVHQG | SEQ ID NO: 111 |
| 112 | RNFPMAQVHQGLMPT | SEQ ID NO: 112 |
| 113 | MAQVHQGLMPTAPPE | SEQ ID NO: 113 |
| 114 | HQGLMPTAPPEDPAV | SEQ ID NO: 114 |
| 115 | MPTAPPEDPAVDLLK | SEQ ID NO: 115 |
| 116 | PPEDPAVDLLKNYMQ | SEQ ID NO: 116 |
| 117 | PAVDLLKNYMQLGKQ | SEQ ID NO: 117 |
| 118 | LLKNYMQLGKQQREK | SEQ ID NO: 118 |
| 119 | YMQLGKQQREKQRES | SEQ ID NO: 119 |
| 120 | GKQQREKQRESREKP | SEQ ID NO: 120 |
| 121 | REKQRESREKPYKEV | SEQ ID NO: 121 |
| 122 | RESREKPYKEVTEDL | SEQ ID NO: 122 |
| 123 | EKPYKEVTEDLLHLN | SEQ ID NO: 123 |
| 124 | KEVTEDLLHLNSLFG | SEQ ID NO: 124 |
| 125 | EDLLHLNSLFGGDQ | SEQ ID NO: 125 |

TABLE 3
One embodiment of an HIV-1 consensus B clade
Gag peptide pool sequence. Each peptide is 15
amino acids in length and overlaps the preceding
peptide by 11 amino acids. Peptide 124 is 12
amino acids in length. The full-length Gag
sequence [SEQ ID NO: 251] is modified from
the HIV sequence database.

| # | PEPTIDE | SEQUENCE ID |
|---|---|---|
| 1 | MGARASVLSGGELDR | SEQ ID NO: 126 |
| 2 | ASVLSGGELDRWEKI | SEQ ID NO: 127 |
| 3 | SGGELDRWEKIRLRP | SEQ ID NO: 128 |
| 4 | LDRWEKIRLRPGGKK | SEQ ID NO: 129 |
| 5 | EKIRLRPGGKKKYKL | SEQ ID NO: 130 |
| 6 | LRPGGKKKYKLKHIV | SEQ ID NO: 131 |
| 7 | GKKKYKLKHIVWASR | SEQ ID NO: 132 |
| 8 | YKLKHIVWASRELER | SEQ ID NO: 133 |
| 9 | HIVWASRELERFAVN | SEQ ID NO: 134 |
| 10 | ASRELERFAVNPGLL | SEQ ID NO: 135 |
| 11 | ELERFAVNPGLLETS | SEQ ID NO: 136 |
| 12 | FAVNPGLLETSEGCR | SEQ ID NO: 137 |
| 13 | PGLLETSEGCRQILG | SEQ ID NO: 138 |
| 14 | ETSEGCRQILGQLQP | SEQ ID NO: 139 |
| 15 | GCRQILGQLQPSLQT | SEQ ID NO: 140 |
| 16 | ILGQLQPSLQTGSEE | SEQ ID NO: 141 |
| 17 | LQPSLQTGSEELRSL | SEQ ID NO: 142 |
| 18 | LQTGSEELRSLYNTV | SEQ ID NO: 143 |
| 19 | SEELRSLYNTVATLY | SEQ ID NO: 144 |
| 20 | RSLYNTVATLYCVHQ | SEQ ID NO: 145 |
| 21 | NTVATLYCVHQRIEV | SEQ ID NO: 146 |
| 22 | TLYCVHQRIEVKDTK | SEQ ID NO: 147 |
| 23 | VHQRIEVKDTKEALE | SEQ ID NO: 148 |
| 24 | IEVKDTKEALEKIEE | SEQ ID NO: 149 |
| 25 | DTKEALEKIEEEQNK | SEQ ID NO: 150 |
| 26 | ALEKIEEEQNKSKKK | SEQ ID NO: 151 |
| 27 | IEEEQNKSKKKAQQA | SEQ ID NO: 152 |
| 28 | QNKSKKKAQQAAADT | SEQ ID NO: 153 |
| 29 | KKKAQQAAADTGNSS | SEQ ID NO: 154 |
| 30 | QQAAADTGNSSQVSQ | SEQ ID NO: 155 |
| 31 | ADTGNSSQVSQNYPI | SEQ ID NO: 156 |
| 32 | NSSQVSQNYPIVQNL | SEQ ID NO: 157 |
| 33 | VSQNYPIVQNLQGQM | SEQ ID NO: 158 |
| 34 | YPIVQNLQGQMVHQA | SEQ ID NO: 159 |
| 35 | QNLQGQMVHQAISPR | SEQ ID NO: 160 |
| 36 | GQMVHQAISPRTLNA | SEQ ID NO: 161 |
| 37 | HQAISPRTLNAWVKV | SEQ ID NO: 162 |
| 38 | SPRTLNAWVKVVEEK | SEQ ID NO: 163 |
| 39 | LNAWVKVVEEKAFSP | SEQ ID NO: 164 |
| 40 | VKVVEEKAFSPEVIP | SEQ ID NO: 165 |
| 41 | EEKAFSPEVIPMFSA | SEQ ID NO: 166 |
| 42 | FSPEVIPMFSALSEG | SEQ ID NO: 167 |
| 43 | VIPMFSALSEGATPQ | SEQ ID NO: 168 |
| 44 | FSALSEGATPQDLNT | SEQ ID NO: 169 |
| 45 | SEGATPQDLNTMLNT | SEQ ID NO: 170 |
| 46 | TPQDLNTMLNTVGGH | SEQ ID NO: 171 |
| 47 | LNTMLNTVGGHQAAM | SEQ ID NO: 172 |
| 48 | LNTVGGHQAAMQMLK | SEQ ID NO: 173 |
| 49 | GGHQAAMQMLKETIN | SEQ ID NO: 174 |
| 50 | AAMQMLKETINEEAA | SEQ ID NO: 175 |
| 51 | QMLKETINEEAAEWD | SEQ ID NO: 176 |
| 52 | ETINEEAAEWDRLHP | SEQ ID NO: 177 |
| 53 | EEAAEWDRLHPVHAG | SEQ ID NO: 178 |
| 54 | EWDRLHPVHAGPIAP | SEQ ID NO: 179 |
| 55 | LHPVHAGPIAPGQMR | SEQ ID NO: 180 |
| 56 | HAGPIAPGQMREPRG | SEQ ID NO: 181 |
| 57 | IAPGQMREPRGSDIA | SEQ ID NO: 182 |
| 58 | QMREPRGSDIAGTTS | SEQ ID NO: 183 |
| 59 | PRGSDIAGTTSTLQE | SEQ ID NO: 184 |
| 60 | DIAGTTSTLQEQIGW | SEQ ID NO: 185 |
| 61 | TTSTLQEQIGWMTNN | SEQ ID NO: 186 |
| 62 | LQEQIGWMTNNPPIP | SEQ ID NO: 187 |
| 63 | IGWMTNNPPIPVGEI | SEQ ID NO: 188 |
| 64 | TNNPPIPVGEIYKRW | SEQ ID NO: 189 |
| 65 | PIPVGEIYKRWIILG | SEQ ID NO: 190 |
| 66 | GEIYKRWIILGLNKI | SEQ ID NO: 191 |
| 67 | KRWIILGLNKIVRMY | SEQ ID NO: 192 |
| 68 | ILGLNKIVRMYSPTS | SEQ ID NO: 193 |
| 69 | NKIVRMYSPTSILDI | SEQ ID NO: 194 |
| 70 | RMYSPTSILDIRQGP | SEQ ID NO: 195 |
| 71 | PTSILDIRQGPKEPF | SEQ ID NO: 196 |
| 72 | LDIRQGPKEPFRDYV | SEQ ID NO: 197 |
| 73 | QGPKEPFRDYVDRFY | SEQ ID NO: 198 |
| 74 | EPFRDYVDRFYKTLR | SEQ ID NO: 199 |
| 75 | DYVDRFYKTLRAEQA | SEQ ID NO: 200 |
| 76 | RFYKTLRAEQASQEV | SEQ ID NO: 201 |
| 77 | TLRAEQASQEVKNWM | SEQ ID NO: 202 |
| 78 | EQASQEVKNWMTETL | SEQ ID NO: 203 |
| 79 | QEVKNWMTETLLVQN | SEQ ID NO: 204 |
| 80 | NWMTETLLVQNANPD | SEQ ID NO: 205 |
| 81 | ETLLVQNANPDCKTI | SEQ ID NO: 206 |
| 82 | VQNANPDCKTILKAL | SEQ ID NO: 207 |
| 83 | NPDCKTILKALGPAA | SEQ ID NO: 208 |
| 84 | KTILKALGPAATLEE | SEQ ID NO: 209 |
| 85 | KALGPAATLEEMMTA | SEQ ID NO: 210 |
| 86 | PAATLEEMMTACQGV | SEQ ID NO: 211 |
| 87 | LEEMMTACQGVGGPG | SEQ ID NO: 212 |
| 88 | MTACQGVGGPGHKAR | SEQ ID NO: 213 |
| 89 | QGVGGPGHKARVLAE | SEQ ID NO: 214 |
| 90 | GPGHKARVLAEAMSQ | SEQ ID NO: 215 |
| 91 | KARVLAEAMSQVTNS | SEQ ID NO: 216 |
| 92 | LAEAMSQVTNSATIM | SEQ ID NO: 217 |
| 93 | MSQVTNSATIMMQRG | SEQ ID NO: 218 |
| 94 | TNSATIMMQRGNFRN | SEQ ID NO: 219 |
| 95 | TIMMQRGNFRNQRKT | SEQ ID NO: 220 |
| 96 | QRGNFRNQRKTVKCF | SEQ ID NO: 221 |
| 97 | FRNQRKTVKCFNCGK | SEQ ID NO: 222 |
| 98 | RKTVKCFNCGKEGHI | SEQ ID NO: 223 |
| 99 | VKCFNCGKEGHIAKN | SEQ ID NO: 224 |
| 100 | NCGKEGHIAKNCRAP | SEQ ID NO: 225 |
| 101 | EGHIAKNCRAPRKKG | SEQ ID NO: 226 |
| 102 | AKNCRAPRKKGCWKC | SEQ ID NO: 227 |
| 103 | RAPRKKGCWKCGKEG | SEQ ID NO: 228 |
| 104 | KKGCWKCGKEGHQMK | SEQ ID NO: 229 |
| 105 | WKCGKEGHQMKDCTE | SEQ ID NO: 230 |
| 106 | KEGHQMKDCTERQAN | SEQ ID NO: 231 |
| 107 | QMKDCTERQANFLGK | SEQ ID NO: 232 |
| 108 | CTERQANFLGKIWPS | SEQ ID NO: 233 |
| 109 | QANFLGKIWPSHKGR | SEQ ID NO: 234 |
| 110 | LGKIWPSHKGRPGNF | SEQ ID NO: 235 |
| 111 | WPSHKGRPGNFLQSR | SEQ ID NO: 236 |
| 112 | KGRPGNFLQSRPEPT | SEQ ID NO: 237 |
| 113 | GNFLQSRPEPTAPPE | SEQ ID NO: 238 |
| 114 | QSRPEPTAPPEESFR | SEQ ID NO: 239 |
| 115 | EPTAPPEESFRFGEE | SEQ ID NO: 240 |
| 116 | PPEESFRFGEETTTP | SEQ ID NO: 241 |
| 117 | SFRFGEETTTPSQKQ | SEQ ID NO: 242 |
| 118 | GEETTTPSQKQEPID | SEQ ID NO: 243 |
| 119 | TTTPSQKQEPIDKEL | SEQ ID NO: 244 |
| 120 | SQKQEPIDKELYPLA | SEQ ID NO: 245 |
| 121 | EPIDKELYPLASLRS | SEQ ID NO: 246 |
| 122 | KELYPLASLRSLFGN | SEQ ID NO: 247 |
| 123 | PLASLRSLFGNDPSS | SEQ ID NO: 248 |
| 124 | LRSLFGNDPSSQ | SEQ ID NO: 249 |

TABLE 4
AN EMBODIMENT OF A FULL-LENGTH SIV GAG SEQUENCE:

| | |
|---|---|
| MGVRNSVLSGKKADELEKIRLRPNGKKKYMLKHV | SEQ ID NO: 250 |
| VWAANELDRFGLAESLLENKEGCQKILSVLAPLV | |
| PTGSENLKSLYNTVCVIWCIHAEEKVKHTEEAKQ | |
| IVQRHLVVETGTTETMPKTSRPTAPSSGRGGNYP | |
| VQQIGGNYVHLPLSPRTLNAWVKLIEEKKFGAEV | |
| VIEEKKFGAEVVPGFQALSEGCTPYDINQMLNCV | |
| GDHQAAMQIIRDIINEEAADWDLQHPQPAPQQGQ | |
| LREPSGSDIAGTTSSVDEQIQWMYRQQNPIPVGN | |
| IYRRWIQLGLQKCVRMYNPTNILDVKQGPKEPFQ | |
| SYVDRFYKSLRAEQTDAAVKNWMTQTLLIQNANP | |
| DCTLLIQNANPDCKLVLKGLGVNPTLEEMLTACQ | |
| GVGGPGQKARLMAEALKEALAPVPIPFAAAQQRG | |
| PRKPIKCWNCGKEGHSARQCRAPRRQGCWKCGKM | |
| DHVMAKCPDRQAGFLGLGPWGKKPRNFPMAQVHQ | |
| GLMPTAPPEDPAVDLLKNYMQLGKQQREKQRESR | |
| EKPREKQRESREKPYKEVTEDLLHLNSLFGGDQ | |

TABLE 5
AN EMBODIMENT OF A FULL-LENGTH HIV-1 GAG SEQUENCE:

| | |
|---|---|
| MGARASVLSGGELDRWEKIRLRPGGKKKYKLKHIV | SEQ ID NO: 251 |
| WASRELERFAVNPGLLETSEGCRQILGQLQPSLQT | |
| GSEELRSLYNTVATLYCVHQRIEVKDTKEALEKIE | |
| EEQNKSKKKAQQAAADTGNSSQVSQNYPIVQNLQG | |
| QMVHQAISPRTLNAWVKVVEEKAFSPEVIPEEKAF | |
| SPEVIPMFSALSEGATPQDLNTMLNTVGGHQAAMQ | |
| MLKETINEEAAEWDRLHPVHAGPIAPGQMREPRGS | |
| DIAGTTSTLQEQIGWMTNNPPIPVGEIYKRWIILG | |
| LNKIVRMYSPTSILDIRQGPKEPFRDYVDRFYKTL | |
| RAEQASQEVKNWMTETLLVQNANPDETLLVQNANP | |
| DCKTILKALGPAATLEEMMTACQGVGGPGHKARVL | |
| AEAMSQVTNSATIMMQRGNFRNQRKTVKCFNCGKE | |
| GHIAKNCRAPRKKGCWKCGKEGHQMKDCTERQANF | |
| LGKIWPSHKGRPGNFLQSRPEPTAPPEESFRFGEE | |
| TTTPSQKQEPIDKELYPLAEPIDKELYPLASLRSL | |
| FGNDPSSQ | |

TABLE 6
LIST OF NON-CONVENTIONAL AMINO ACIDS

| Non-conventional amino acid | Non-conventional amino acid |
|---|---|
| α-aminobutyric acid | L-N-methylalanine |
| α-amino-α-methylbutyrate | L-N-methylarginine |
| aminocyclopropane-carboxylate | L-N-methylasparagine |
| aminoisobutyric acid | L-N-methylaspartic acid |
| aminonorbornyl-carboxylate | L-N-methylcysteine |
| cyclohexylalanine | L-N-methylglutamine |
| cyclopentylalanine | L-N-methylglutamic acid |
| L-N-methylisoleucine | L-N-methylhistidine |
| D-alanine | L-N-methylleucine |
| D-arginine | L-N-methyllysine |
| D-aspartic acid | L-N-methylmethionine |
| D-cysteine | L-N-methylnorleucine |
| D-glutamate | L-N-methylnorvaline |
| D-glutamic acid | L-N-methylornithine |
| D-histidine | L-N-methylphenylalanine |
| D-isoleucine | L-N-methylproline |
| D-leucine | L-N-methylserine |
| D-lysine | L-N-methylthreonine |
| D-methionine | L-N-methyltryptophan |
| D-ornithine | L-N-methyltyrosine |
| D-phenylalanine | L-N-methylvaline |
| D-proline | L-N-methylethylglycine |
| D-serine | L-N-methyl-t-butylglycine |
| D-threonine | L-norleucine |
| D-tryptophan | L-norvaline |
| D-tyrosine | α-methyl-aminoisobutyrate |
| D-valine | α-methyl-γ-aminobutyrate |
| D-α-methylalanine | α-methylcyclohexylalanine |
| D-α-methylarginine | α-methylcyclopentylalanine |
| D-α-methylasparagine | α-methyl-α-naphthylalanine |
| D-α-methylaspartate | α-methylpenicillamine |
| D-α-methylcysteine | N-(4-aminobutyl)glycine |
| D-α-methylglutamine | N-(2-aminoethyl)glycine |
| D-α-methylhistidine | N-(3-aminopropyl)glycine |
| D-α-methylisoleucine | N-amino-α-methylbutyrate |
| D-α-methylleucine | α-naphthylalanine |
| D-α-methyllysine | N-benzylglycine |
| D-α-methylmethionine | N-(2-carbamylethyl)glycine |
| D-α-methylornithine | N-(carbamylmethyl)glycine |
| D-α-methylphenylalanine | N-(2-carboxyethyl)glycine |
| D-α-methylproline | N-(carboxymethyl)glycine |
| D-α-methylserine | N-cyclobutylglycine |
| D-α-methylthreonine | N-cycloheptylglycine |
| D-α-methyltryptophan | N-cyclohexylglycine |
| D-α-methyltyrosine | N-cyclodecylglycine |
| L-α-methylleucine | L-α-methyllysine |
| L-α-methylmethionine | L-α-methylnorleucine |
| L-α-methylnorvaline | L-α-methylornithine |
| L-α-methylphenylalanine | L-α-methylproline |
| L-α-methylserine | L-α-methylthreonine |
| L-α-methyltryptophan | L-α-methyltyrosine |
| L-α-methylvaline | L-N-methylhomophenylalanine |
| N-(N-(2,2-diphenylethyl | N-(N-(3,3-diphenylpropyl |
| carbamylmethyl)glycine | carbamylmethyl)glycine |
| 1-carboxy-1-(2,2-diphenyl-ethyl | |
| amino)cyclopropane | |

TABLE 7
STATISTICAL ANALYSIS ON VL AND SURVIVAL

| | | | Reduction in | 2 sided | Reduction in | 2 sided | Reduction in | 2 sided | Survival 1 |
|---|---|---|---|---|---|---|---|---|---|
| Animals | | | VL 10 weeks | t-test | VL 6 months | t-test | VL 1 year | t-test | year off ART |
| analyzed | Comparison | n | off ART* | (p-value) | off ART | (p-value) | off ART** | (p-value) | (p-value)† |
| VL undetectable | OPAL- Gag + | 16 vs 10 | 0.50 (0.73) | 0.084 | 0.64 (0.93) | 0.028 | 0.80 (0.98) | 0.019 | 0.053 |
| at week | All vs controls | | | | | | | | |
| 10 (n = 26) | OPAL-All vs controls | 8 vs 10 | 0.42 (0.74) | 0.136 | 0.61 (0.90) | 0.114 | 0.79 (0.88) | 0.066 | NA†† |
| | OPAL-Gag vs controls | 8 vs 10 | 0.57 (0.71) | 0.262 | 0.67 (0.95) | 0.080 | 0.81 (1.07) | 0.069 | 0.212 |
| All animals, | OPAL-Gag + | 21 vs 11 | 0.47 (0.54) | 0.050 | 0.61 (0.66) | 0.016 | 0.74 (0.66) | 0.011 | 0.020 |
| adjusted for | All vs controls | | | | | | | | |
| Mane-A*10 | OPAL-All vs controls | 11 vs 11 | 0.51 (0.53) | 0.072 | 0.60 (0.64) | 0.040 | 0.71 (0.60) | 0.023 | 0.054 |
| status and | OPAL-Gag vs controls | 10 vs 11 | 0.44 (0.54) | 0.116 | 0.63 (0.69) | 0.032 | 0.77 (0.72) | 0.035 | 0.022 |
| VL at week | | | | | | | | | |
| 10 (n = 32) | | | | | | | | | |

*VL values reductions are log₁₀ copies/ml compared to controls. Values shown reflect time-weighted AUC VL between vaccinated animals and controls after coming off ART, and absolute mean reduction at end of the period in parentheses.
**12 animals died after week 41; the mean of the 2 last VL observations were carried forward were used to estimate differences in VL to week 64.
†Survival p-value reflect Cox-regression analysis.
††None of 8 OPAL-All vaccinated animals that had VL undetectable on ART died vs 5 of 10 controls - this comparison did not permit an estimate of significance of this comparison.

### BIBLIOGRAPHY

1. Markowitz, M. et al. Discontinuation of antiretroviral therapy commenced early during the course of human immunodeficiency virus type 1 infection, with or without adjunctive vaccination. J Infect Dis 186, 634-643 (2002).
2. Tubiana, R. et al. Therapeutic immunization with a human immunodeficiency virus (HIV) type 1-recombinant canarypox vaccine in chronically HIV-infected patients: The Vacciter Study (ANRS 094). Vaccine 23, 4292-4301 (2005).
3. Kahn, J. O., Cherng, D. W., Mayer, K., Murray, H. & Lagakos, S. Evaluation of HIV-1 immunogen, an immunologic modifier, administered to patients infected with HIV having 300 to 549×10(6)/L CD4 cell counts: A randomized controlled trial. Jama 284, 2193-2202 (2000).
4. Katlama, C. et al. A Phase II, Randomized, Placebo-controlled Study to Evaluate the Immunogenicity of 2 Schedules of an Homologous Prime-boost with the ALVAC-HIV vCP1452 in Chronically HIV-infected Patients (MANON 02. 14th Conference on Retroviruses and Opportunistic infections, Feb. 25-28 2007, Los Angeles Abstract 486 (2007).
5. Lu, W., Wu, X., Lu, Y., Guo, W. & Andrieu, J. M. Therapeutic dendritic-cell vaccine for simian AIDS. Nat Med 9, 27-32 (2003).
6. Lu, W., Arraes, L. C., Ferreira, W. T. & Andrieu, J. M. Therapeutic dendritic-cell vaccine for chronic HIV-1 infection. Nat Med 10, 1359-1365 (2004).
7. Chea, S., Dale, C. J., De Rose, R., Ramshaw, I. A. & Kent, S. J. Enhanced cellular immunity in macaques following a novel peptide immunotherapy. J Virol 79, 3748-3757 (2005).
8. Stratov, I., Dale, C. J., Chea, S., McCluskey, J. & Kent, S. J. Induction of T-cell immunity to antiretroviral drug-resistant human immunodeficiency virus type 1. J Virol 79, 7728-7737 (2005).
9. Batten, C. J. et al. Comparative Evaluation of Simian, Simian-Human, and Human Immunodeficiency Virus Infections in the Pigtail Macaque (Macaca nemestrina) Model. AIDS Res Hum Retroviruses 22, 580-588 (2006).
10. Reimann, K. A. et al. Pathogenicity of simian-human immunodeficiency virus SHIV-89.6P and SIVmac is attenuated in cynomolgus macaques and associated with early T-lymphocyte responses. J Virol 79, 8878-8885 (2005).
11. Smith, M. Z. et al. Analysis of Pigtail Macaque Major Histocompatibility Complex Class I Molecules Presenting Immunodominant Simian Immunodeficiency Virus Epitopes. J Virol 79, 684-695 (2005).
12. Hel, Z. et al. Viremia control following antiretroviral treatment and therapeutic immunization during primary SIV251 infection of macaques. Nat Med 6, 1140-1146. (2000).
13. Cline, A. N., Bess, J. W., Piatak, M., Jr. & Lifson, J. D. Highly sensitive SIV plasma viral load assay: practical considerations, realistic performance expectations, and application to reverse engineering of vaccines for AIDS. J Med Primatol 34, 303-312 (2005).
14. von Gegerfelt, A. S. et al. Long-lasting decrease in viremia in macaques chronically infected with simian immunodeficiency virus SIVmac251 after therapeutic DNA immunization. J Virol 81, 1972-1979 (2007).
15. Lisziewicz, J. et al. Control of viral rebound through therapeutic immunization with DermaVir. Aids 19, 35-43 (2005).
16. Rosenberg, E. S. et al. Vigorous HIV-1-specific CD4+ T cell responses associated with control of viremia. Science 278, 1447-1450 (1997).
17. Mattapallil, J. J. et al. Massive infection and loss of memory CD4+ T cells in multiple tissues during acute SIV infection. Nature 434, 1093-1097 (2005).
18. O'Brien, W. A. et al. Changes in plasma HIV-1 RNA and CD4+ lymphocyte counts and the risk of progression to AIDS. Veterans Affairs Cooperative Study Group on AIDS. N Engl J Med 334, 426-431 (1996).
19. De Rose, R. et al. Comparative Efficacy of Subtype AE Simian-Human Immunodeficiency Virus Priming and Boosting Vaccines in Pigtail Macaques. J Virol 81, 292-300 (2007).
20. Kiepiela, P. et al. CD8(+) T-cell responses to different HIV proteins have discordant associations with viral load. Nat Med 13, 46-53 (2007).
21. Pratt, B. F. et al. MHC class I allele frequencies in pigtail macaques of diverse origin. Immunogenetics 58, 995-1001 (2006).
22. Smith, M. Z. et al. The pigtail macaque MHC class I allele Mane-A*10 presents an immundominant SIV Gag epitope: identification, tetramer development and implications of immune escape and reversion. J Med Primatol 34, 282-293 (2005).
23. Lori, F. et al. Control of SIV rebound through structured treatment interruptions during early infection. Science 290, 1591-1593 (2000).
24. Villinger, F. et al. Adoptive transfer of simian immunodeficiency virus (SIV) naive autologous CD4(+) cells to macaques chronically infected with SIV is sufficient to induce long-term nonprogressor status. Blood 99, 590-599 (2002).
25. Shen, A. et al. Resting CD4+ T lymphocytes but not thymocytes provide a latent viral reservoir in a simian immunodeficiency virus-Macaca nemestrina model of human immunodeficiency virus type 1-infected patients on highly active antiretroviral therapy. J Virol 77, 4938-4949 (2003).
26. Dale, C. J. et al. Chimeric human papilloma virus-simian/human immunodeficiency virus virus-like-particle vaccines: immunogenicity and protective efficacy in macaques. Virology 301, 176-187 (2002).
27. Fernandez, C. S. et al. Rapid viral escape at an immunodominant simian-human immunodeficiency virus cytotoxic T-lymphocyte epitope exacts a dramatic fitness cost. J Virol 79, 5721-5731 (2005).
28. Matthews, J. N., Altman, D. G., Campbell, M. J. & Royston, P. Analysis of serial measurements in medical research. Bmj 300, 230-235 (1990).
29. Fuller, D. H. et al. DNA immunization in combination with effective antiretroviral drug therapy controls viral rebound and prevents simian AIDS after treatment is discontinued. Virology 348, 200-215 (2006).

1. A method for treating or preventing a lentivirus infection in a subject, the method comprising increasing in the subject the number of Gag-specific antigen-presenting cells or Gag-specific antigen-presenting cell precursors, which present on their surface at least one peptide that comprises an amino acid sequence corresponding to a portion of a Gag polypeptide, wherein the Gag-specific antigen-presenting cells or the Gag-specific antigen-presenting cell precursors are produced by contacting antigen-presenting cells or antigen-presenting cell precursors with a composition that consists essentially of a plurality of peptides for a time and under conditions sufficient for the peptides, or processed forms of the peptides, to be presented by the antigen-presenting cells or by the precursors on their surface, wherein individual peptides of the composition comprise different portions of an amino acid sequence corresponding to a Gag polypeptide and optionally display partial sequence identity or similarity to at least one other peptide of the plurality of peptides.

2. A method according to claim 1, wherein the subject is administered the Gag-specific antigen-presenting cells or the Gag-specific antigen-presenting cell precursors.

3. A method according to claim 1, wherein the subject is administered the composition.

4. A method according to claim 3, wherein the peptides are contained or otherwise associated with a particle.

5. A method according to claim 4, wherein the particle is selected from the group consisting of liposomes, micelles, lipidic particles, ceramic/inorganic particles and polymeric particles.

6. A method according to claim 1, wherein the method excludes administering to the subject (1) a peptide that comprises an amino acid sequence corresponding to a portion of a non-Gag lentivirus polypeptide, or (2) an antigen-presenting cell that has been contacted with a peptide according to (1).

7. A method according to claim 1, wherein the antigen presenting cells are selected from the group consisting of dendritic cells, macrophages and Langerhans cells.

8-10. (canceled)

11. A method according to claim 1, wherein the lentivirus is selected from the group consisting of human immunodeficiency virus (HIV) and simian immunodeficiency virus (SIV).

12. A method according to claim 1, wherein the partial sequence identity or similarity is contained at one or both ends of an individual peptide.

13. A method according to claim 12, wherein at least 4 contiguous amino acid residues are present at one or both of these ends, whose sequence is identical or similar to an amino acid sequence contained within at least one other of the peptides.

14. A method according to claim 12, wherein the peptide is at least 6 amino acid residues in length.

15-19. (canceled)

20. A method according to claim 12, wherein the peptide sequences are derived from at least about 30% of the sequence corresponding to the Gag polypeptide.

21. A method according to claim 12, wherein the plurality of peptides comprises peptides from two or more different Gag polypeptides.

22-38. (canceled)

39. A composition consisting essentially of antigen-presenting cells or antigen-presenting cell precursors which have been contacted with a composition that consists essentially of a plurality of peptides for a time and under conditions sufficient for the peptides, or processed forms of the peptides, to be presented by the antigen-presenting cells or by the precursors on their surface, wherein individual peptides of the composition comprise different portions of an amino acid sequence corresponding to a Gag polypeptide and optionally display partial sequence identity or similarity to at least one other peptide of the plurality of peptides.

40. A composition according to claim 39, wherein the antigen-presenting cells or antigen-presenting cell precursors are in the form of a substantially purified population of antigen-presenting cells or precursors.

41. A composition according to claim 39, wherein the antigen-presenting cells or antigen-presenting cell precursors are in the form of a heterogeneous population of antigen-presenting cells or precursors.

42. A composition according to claim 41, wherein the heterogeneous population of antigen-presenting cells or their precursors is selected from the group consisting of blood and peripheral blood mononuclear cells.

43. A composition according to claim 39, wherein the antigen-presenting cells or their precursors are selected from the group consisting of monocytes, macrophages, cells of myeloid lineage, B cells, dendritic cells and Langerhans cells.

44. A composition according to claim 39, wherein the antigen-presenting cells or their precursors are in the form of an uncultured population of antigen-presenting cells or their precursors.

45. A composition according to claim 44, wherein the population is homogeneous.

46. A composition according to claim 44, wherein the population is heterogeneous.

47. A composition according to claim 44, wherein the population is selected from the group consisting of whole blood, fresh blood, or fractions thereof, peripheral blood mononuclear cells, buffy coat fractions of whole blood, packed red cells, irradiated blood, dendritic cells, monocytes, macrophages, neutrophils, lymphocytes, natural killer cells and natural killer T cells.

48. A composition according to claim 44, wherein the population has not been subjected to activating conditions.

49. A composition according to claim 39, excluding antigen-presenting cells that present on their surface peptides that comprise amino acid sequences corresponding to portions of non-Gag polypeptides.

50-66. (canceled)
